(19) United States
(12) Patent Application Publication
(10) Pub. No.: US 2024/0087733 A1
(43) Pub. Date: Mar. 14, 2024

(54) DEPLOYMENT STATIONS FOR EMERGENCY MEDICAL TREATMENT AND GUIDANCE APPARATUSES

(71) Applicant: ZOLL Medical Corporation, Chelmsford, MA (US)

(72) Inventors: Matthew J. Grey, Fairport, NY (US); Adrian A. Alvarez, Chelmsford, MA (US); Kristopher M. Edgell, Shrevport, LA (US); Frederick K. Newey, Chelmsford, MA (US); Gary A. Freeman, Waltham, MA (US)

(21) Appl. No.: 18/262,881

(22) PCT Filed: Jan. 25, 2022
(86) PCT No.: PCT/US22/13700

§ 371 Date: Jul. 25, 2023

Related U.S. Application Data
(60) Provisional application No. 63/141,645, filed on Jan. 26, 2021.

(51) Int. Cl. G16H 40/40; A61N 1/39; G16H 20/30

(52) U.S. Cl. CPC G16H 40/40; A61N 1/3904; A61N 1/3993; G16H 20/30; G06K 19/0723

(57) ABSTRACT

In one aspect, a deployment station for portable emergency medical treatment and guidance apparatuses is provided. The deployment station can include a first storage section for holding at least one emergency medical treatment and guidance apparatus that includes a plurality of medical supplies; a second storage section for holding at least one defibrillator including electronic circuitry to administer electrotherapy; a housing containing the first storage section and the second storage section, the housing including: a readiness indicator for indicating a readiness of the at least one emergency medical treatment and guidance apparatus and the at least one defibrillator based on obtained readiness information; and at least one processor and memory mechanically coupled to the housing and communicatively coupled to the readiness indicator.

### CROSS REFERENCE TO RELATED APPLICATIONS

The present application claims priority to U.S. Provisional Patent Application No. 63/141,645 entitled “Deployment Stations For Emergency Medical Treatment And Guidance Apparatuses” filed Jan. 26, 2021, which is hereby incorporated by reference in its entirety.

### FIELD

The present disclosure relates to deployment stations for emergency medical treatment and guidance apparatuses, and more particularly, to deployment stations that include readiness indicators for indicating a readiness of at least one emergency medical treatment and guidance apparatus and at least one defibrillator.

### BACKGROUND

Various different types of medical first aid kits exist to supply first aid to an injured person. Some such first aid kits also provide written and/or audible instructions for how to treat patients, using the medical supplies contained within the first aid kits. First aid kits may be stored at places where people congregate and therefore medical emergencies are likely to occur (e.g., at workplaces, stores, and schools). In an emergency, a caregiver may locate a portable first aid kit and carry the portable first aid kit to a location of a patient. The caregiver may use the supplies in the portable first aid kit to treat one or more medical emergencies from which the patient may be suffering.

### SUMMARY

In one aspect, a deployment station for emergency medical treatment and guidance apparatuses include a first storage section for holding at least one emergency medical treatment and guidance apparatus including a plurality of medical supplies; a second storage section for holding at least one medical defibrillator including electronic circuitry to administer electrotherapy; and a housing containing the first storage section and the second storage section.

In some implementations, the housing includes a readiness indicator disposed on an exterior surface of the housing for indicating a readiness of the at least one emergency medical treatment and guidance apparatus and the at least one defibrillator based on obtained readiness information; and at least one processor and memory mechanically coupled to the housing and communicatively coupled to the readiness indicator.

In some implementations, the at least one processor and memory is configured for: obtaining the readiness information of the at least one emergency medical treatment and guidance apparatus and the at least one defibrillator; determining readiness of the at least one emergency medical treatment and guidance apparatus and the at least one defibrillator based on the obtained readiness information; updating the readiness indicator based on the determined readiness; and transmitting a readiness signal representing the readiness of the at least one emergency medical treatment and guidance apparatus and the at least one defibrillator to a server for maintaining a database of the readiness of the at least one emergency medical treatment and guidance apparatus and the at least one defibrillator within the deployment station.

In some implementations, the housing further includes at least one sensor for obtaining the readiness information of the at least one emergency medical treatment and guidance apparatus and the at least one defibrillator. In this scenario, the at least one processor and memory is configured for receiving a signal from the at least one sensor. In some scenarios, the at least one processor and memory is configured for identifying the at least one emergency medical treatment and guidance and the at least one defibrillator based on identification information obtained from the at least one sensor. In some scenarios, the at least one sensor includes a radio frequency identification (RFID) reader and each of the at least one emergency medical treatment and guidance apparatuses and each of the at least one defibrillators includes a respective unique RFID tag readable by the RFID reader. In this scenario, the identification information can include a serial number corresponding to the at least one emergency medical treatment and guidance apparatus and/or the at least one defibrillator, and the readiness information can include an expiration date corresponding to the at least one emergency medical treatment and guidance apparatus and/or the at least one defibrillator. In some scenarios, the expiration date corresponding to the at least one defibrillator includes an expiration date of electrodes for administering electrotherapy. In some scenarios, the expiration date corresponding to the at least one emergency medical treatment and guidance apparatus includes an expiration date of at least one of the plurality of medical supplies.

In some implementations, determining readiness of the at least one emergency medical treatment and guidance apparatus and the at least one defibrillator includes determining whether a battery of the at least one emergency medical treatment and guidance apparatus or the at least one defibrillator has a battery charge above a pre-determined threshold. In this scenario, the battery can include a battery of a touch screen user interface of the at least one emergency medical treatment and guidance apparatus.

In some implementations, determining readiness of the at least one emergency medical treatment and guidance apparatus and the at least one defibrillator includes determining how an inventory of medical supplies within the at least one emergency medical treatment and guidance apparatus compares with an expected inventory representing a fully stocked emergency medical treatment and guidance apparatus. In this scenario, the expected inventory can be retrieved from the database. In some scenarios, determining how the inventory of the medical supplies within the at least one emergency medical treatment and guidance apparatus compares with the expected inventory representing a fully stocked emergency medical treatment and guidance apparatus includes determining that the inventory of the medical supplies matches the expected inventory. In some scenarios, determining how the inventory of the medical supplies within the at least one emergency medical treatment and guidance apparatus compares with the expected inventory representing a fully stocked emergency medical treatment and guidance apparatus includes determining that at least a portion of the inventory of the medical supplies is missing.

In some implementations, determining readiness of the at least one emergency medical treatment and guidance apparatus and the at least one defibrillator includes receiving an indication from the at least one defibrillator regarding whether the at least one defibrillator has passed a self-diagnostic test.

In some implementations, determining readiness of the at least one emergency medical treatment and guidance apparatus and the at least one defibrillator includes querying the database to determine if a status report indicates a use of the at least one emergency medical treatment and guidance apparatus.

In some implementations, the at least one defibrillator includes an automated external defibrillator (AED) and the AED includes electronic circuitry for performing a self-diagnostic capability to determine a status of the AED. In this scenario, the determined status can be transmitted to the at least one processor of the deployment station and the readiness of the at least one emergency medical treatment and guidance apparatus and the at least one defibrillator can be based on the determined status of the AED.

In some implementations, the housing further includes an access door mechanically hinged to the housing and lockable using a locking mechanism. In some scenarios, the access door prevents access to the at least one emergency medical treatment and guidance apparatus and the at least one defibrillator when in a locked configuration and does not prevent access in an unlocked configuration. In some scenarios, the locking mechanism is unlocked when a user's identification has been verified with a list of authorized users stored in the database. In this scenario, the user's identification can be represented by a passcode, an access card, or an RFID tag of the user.

In some implementations, the housing further includes a charging system configured to charge the at least one emergency medical treatment and guidance apparatus or the at least one defibrillator. In this scenario, the at least one processor and memory is configured for receiving a battery status of the at least one emergency medical treatment and guidance apparatus or the at least one defibrillator. In this scenario, the readiness is further based on the received battery status.

In some implementations, the housing further includes an environmental controller configured to maintain a target temperature and a target humidity of an interior volume of the housing using a heater and a dehumidifier, respectively. In this scenario, the at least one processor and memory is configured for receiving a measured temperature and a measured humidity of the interior volume of the housing. In this scenario, the readiness can be further based on the received temperature and the received humidity. In some scenarios, the readiness can be further based on whether the measured temperature and the measured humidity are outside of target pre-determined ranges for the at least one emergency medical treatment and guidance apparatus and/or the at least one defibrillator. In some scenarios, the readiness can be further based on whether the measured temperature and the measured humidity are outside of target pre-determined ranges for at least one pharmaceutical within the at least one emergency medical treatment and guidance apparatus. In some scenarios, the readiness can be further based on whether the measured temperature and the measured humidity are outside of target pre-determined ranges for at least one electrode of the at least one defibrillator.

In some implementations, the housing further includes a user interface configured to display a machine-readable code and includes an interactive query to a user. In some scenarios, the machine-readable code includes a barcode or a QR code. In some scenarios, the machine-readable code represents the readiness of the at least one emergency medical treatment and guidance apparatus and the at least one defibrillator. In some scenarios, the machine-readable code encodes a hyperlink such that when imaged by a mobile device the hyperlink directs the mobile device to retrieve contact information from the database and display information related to whom to contact to obtain access to the deployment station. In some scenarios, the machine-readable code encodes a hyperlink such that when imaged by a mobile device the hyperlink directs the mobile device to retrieve connection information from the database and display information related to which devices are in network communication with the deployment station. In some scenarios, the devices related to the displayed information include the at least one emergency medical treatment and guidance apparatus, the at least one defibrillator, and the server. In some scenarios, the machine-readable code encodes a hyperlink such that when imaged by a mobile device the hyperlink directs the mobile device to retrieve readiness information from the database and display information related to the readiness of the at least one emergency medical treatment and guidance apparatus or the at least one defibrillator. In some scenarios, the interactive query is provided for guiding the user in determining which of the at least one emergency medical treatment and guidance apparatus and the at least one defibrillator is needed for medical treatment based on a condition of a patient. In some scenarios, the at least one processor and memory is further configured for recommending one of the at least one emergency medical treatment and guidance apparatus and/or one of the at least one defibrillator based on the query.

In some implementations, the housing further includes a fire extinguisher and the at least one processor and memory is configured for receiving a fire extinguisher signal from at least one sensor of the fire extinguisher. In this scenario, the fire extinguisher signal represents that the fire extinguisher is present within the deployment station. In this scenario, the readiness can be further based on the fire extinguisher signal.

In some implementations, the housing further includes a first mount located within the first storage section for releasably mounting the at least one emergency medical treatment and guidance apparatus within the first storage section, and a second mount located within the second storage section for releasably mounting the at least one defibrillator within the second storage section.

In some implementations, each of the first storage section and the second storage section include an access door. In this scenario, each access door can include a display for presenting a machine-readable code and a storage section readiness indicator. In this scenario, the display can be configured to display the machine-readable code encoding a hyperlink such that when imaged by a mobile device the hyperlink directs the mobile device to retrieve readiness information from the database and display information related to the readiness of the at least one emergency medical treatment and guidance apparatus contained within the first storage section and/or the readiness of the at least one defibrillator contained within the second storage section.

In some implementations, the housing further includes a return chute for depositing used emergency medical treatment and guidance apparatuses and used defibrillators into a storage container within the housing of the deployment station.

In some implementations, the at least one processor and memory is further configured for reading each of at least one RFID tag of a used emergency medical treatment and guidance apparatus and/or a used defibrillator upon being returned to the deployment station, and identifying the used emergency medical treatment and guidance apparatus and/or used defibrillator based on at least one of the at least one RFID tag. In some scenarios, the at least one processor and memory is further configured for transmitting information to the server that the used emergency medical treatment and guidance apparatus and/or the used defibrillator is located within the deployment station. In this scenario, the server can be configured to update location information within the database.

In some implementations, the at least one processor and memory is further configured for receiving information from the database representing expected RFID tags associated with a used emergency medical treatment and guidance apparatus and/or a used defibrillator upon being returned to the deployment station, and determining whether all RFID tags are present based on comparing the read information of the RFID tags with the received information representing the expected RFID tags. In this scenario, the readiness can be based on whether all RFID tags are determined to be present.

In some implementations, the at least one processor and memory is further configured for measuring a weight of a used emergency medical treatment and guidance apparatus and/or a used defibrillator using a weight scale of the deployment station upon being returned to the deployment station, and determining whether the weight is within a pre-determined range of an expected weight based on comparing the weight with an expected weight of the used emergency medical treatment and guidance apparatus and/or used defibrillator. In this scenario, the readiness can be based on whether the weight is determined to be within the pre-determined range.

In some implementations, the at least one processor and memory is further configured for querying the database to determine whether a used emergency medical treatment and guidance apparatus and/or a used defibrillator was used in a medical emergency upon being returned to the deployment station. In this scenario, the readiness can be based on whether used emergency medical treatment and guidance apparatus and/or used defibrillator was used in a medical emergency. In some scenarios, the determination of whether the used emergency medical treatment and guidance apparatus and/or used defibrillator was used in the medical emergency is performed by the server and determined based on a status report associated with the used emergency medical treatment and guidance apparatus and/or used defibrillator.

In some implementations, the at least one processor and memory is further configured for transmitting a signal representing the readiness to a mobile device such that the signal causes the mobile device to trigger a mobile alert indicating the readiness of the at least one emergency medical treatment and guidance apparatus and/or the at least one defibrillator.

In some implementations, the first storage section includes the at least one emergency medical treatment and guidance apparatus containing a plurality of medical supplies and the second storage section includes the at least one defibrillator including electronic circuitry to administer electrotherapy. In some scenarios, the plurality of medical supplies include at least one tourniquet, at least one chest seal, and at least one pair of gloves. In this scenario, the at least one emergency medical treatment and guidance apparatus can be configured to provide an interactive query flow of medical instructions using at least some of the plurality of medical supplies. In some scenarios, the electronic circuitry to administer electrotherapy includes at least one electrode and at least one high-voltage capacitor to administer electrotherapy. In some scenarios, the at least one emergency medical treatment and guidance apparatus and the at least one defibrillator each include an individual readiness indicator.

In some implementations, the housing further includes a camera operable to obtain an image of individual readiness indicators of the at least one emergency medical treatment and guidance apparatus and/or the at least one defibrillator. In this scenario, the at least one processor can be operable to process the image to determine an imaged individual readiness of the respective at least one emergency medical treatment and guidance apparatus and/or the at least one defibrillator such that the readiness of the respective at least one emergency medical treatment and guidance apparatus and/or the at least one defibrillator is based on the imaged individual readiness. In some scenarios, the housing further includes a light operable to illuminate the at least one emergency medical treatment and guidance apparatus and/or the at least one defibrillator when the camera obtains an image.

In some implementations, the housing further includes a microphone and the at least one emergency medical treatment and guidance apparatus and/or the at least one defibrillator includes a speaker and circuitry for transmitting an audible signal to the microphone. In some scenarios, the at least one processor and memory is further configured for receiving the audible signal from the microphone. In this scenario, the audible signal can encode an audible message about the readiness of the at least one emergency medical treatment and guidance apparatus and/or at least one defibrillator. In some scenarios, the at least one processor and memory is further configured for decoding the audible message. In some scenarios, the readiness can be further based on the decoded audible message. In some scenarios, the at least one processor and memory is further configured for receiving the audible signal from the microphone. In this scenario, the audible signal can encode an audible message about the usage of the at least one emergency medical treatment and guidance apparatus and/or at least one defibrillator. In some scenarios, the at least one processor and memory is further configured for decoding the audible message and transmitting a request to the server to update the database based on the decoded audible message. In some scenarios, the audible signal can encode an audible message about missing items of at least one emergency medical treatment and guidance apparatus and/or at least one defibrillator. In some scenarios, the at least one processor and memory is further configured for decoding the audible message and transmitting a request to the server to update the database based on the decoded audible message. In some scenarios, the audible signal is frequency encoded or encoded using a Morse code scheme. In some scenarios, a first frequency indicates that the at least one defibrillator is ready and a second frequency indicates that the at least one defibrillator is not ready.

In some implementations, the readiness indicator is a first readiness indicator and the deployment station further includes a second readiness indicator disposed on the exterior surface of the housing. In some scenarios, updating the readiness indicator based on the determined readiness includes updating the first readiness indicator based on a portion of the determined readiness relating to the determined readiness of a tablet device of the at least one emergency medical treatment and guidance apparatus and updating the second readiness indicator based on a remainder portion of the determined readiness.

### BRIEF DESCRIPTIONS OF THE DRAWINGS

Many advantages of the present disclosure will be apparent to those skilled in the art with a reading of this specification in conjunction with the attached drawings, wherein like reference numerals are applied to like elements and wherein:

FIG. 1 shows a readiness management system of deployment stations with emergency medical treatment and guidance apparatuses and defibrillators in accordance with some embodiments;

FIGS. 2A and 2B show a schematic of a deployment station in accordance with some embodiments with an access door in a closed and open configuration, respectively;

FIG. 2C shows a wiring diagram schematic of a deployment station in accordance with some embodiments;

FIG. 3 is a flowchart of a method performed by a deployment station in accordance with some embodiments;

FIGS. 4A and 4B show a defibrillator with a readiness indicator in accordance with some embodiments; FIG. 4A is a front plan view and FIG. 4B is a schematic view of the defibrillator;

FIGS. 5A and 5B show an emergency medical treatment and guidance apparatus with a readiness indicator in accordance with some embodiments; FIG. 5A is a perspective view and FIG. 5B is a schematic view of the emergency medical treatment and guidance apparatus;

FIG. 6 shows an emergency medical treatment and guidance apparatus with a user interface and a readiness indicator and in accordance with some embodiments;

FIGS. 7A and 7B show an emergency medical treatment and guidance apparatus with a readiness indicator and in accordance with some embodiments;

FIG. 8 shows a schematic of a deployment station in accordance with some embodiments with a return chute and access door for returning used emergency medical treatment and guidance apparatuses and used defibrillators;

FIG. 9 shows a flowchart of a deployment station in accordance with some embodiments for returning used emergency medical treatment and guidance apparatuses and used defibrillators;

FIG. 10 shows a schematic of a deployment station in accordance with some embodiments with a multiple compartments for individually storing emergency medical treatment and guidance apparatuses and defibrillators;

FIG. 11 shows a screenshot of an application on a deployment station as part of the system of FIG. 1 in accordance with some embodiments;

FIGS. 12A, 12B, 12C, and 12D show screenshots of an application on a user's mobile device as part of the system of FIG. 1 in accordance with some embodiments;

FIGS. 13A, 13B, 13C, 13D, 13E, and 13F show screenshots of an application on an inspector's mobile device or tablet as part of the system of FIG. 1 for inventory management of one of more deployment stations in accordance with some embodiments;

FIGS. 14A, 14B, and 14C show screenshots of an application on an inspector's computational device as part of the system of FIG. 1 for inventory management of one of more deployment stations in accordance with some embodiments; and

FIG. 15 is a block diagram of computer systems forming part of the system of FIG. 1 in according to some embodiments.

### DETAILED DESCRIPTION

Embodiments of the present disclosure relate to a deployment station employed as a storage system that houses one or more emergency medical treatment and guidance apparatuses and defibrillators and also provides monitoring capability (e.g., via cameras, microphones, RF communication, among others) for the emergency medical treatment and guidance apparatus(es) and defibrillator(s) to ensure all aspects of these devices are fully functional and ready when called upon for use in a medical emergency. When these devices are ready, a lay rescuer (e.g., a rescuer with little to no medical experience) need not worry that a critical medical item or functionality is missing or broken.

For example, managing the inventory and readiness of each emergency medical treatment and guidance apparatus and each defibrillator contained within the deployment station is especially important in a pre-hospital setting (e.g., schools, offices, airports, among others). In this setting, the focus is on helping the lay rescuers determine which medical device/item is needed for a particular medical emergency, providing a ready medical device/medical item to lay rescuers as quickly as possible, and guiding the lay rescuers on how to use the medical devices and/or supplies associated with the device. In this setting, it cannot be assumed that a medical doctor is immediately available to provide medical attention, and it cannot be assumed that the lay person will know how to treat the medical emergency, let alone which medical supplies are required and/or how to actually perform the treatment.

For example, an emergency medical treatment and guidance apparatus, which is a portable kit for use with electronic interactive guidance, houses multiple medical supplies (e.g., tourniquets, gauze, pharmaceuticals, among others) and enables lay rescuers to administer urgent medical treatment. However, an emergency medical treatment and guidance apparatus with missing medical supplies can be extremely problematic, leading to a matter of life of death, especially when the lay person is expecting a medical item to be present and is in urgent need of the medical item. Therefore, increasing the likelihood that the emergency medical treatment and guidance apparatus is ready (e.g., that it is fully stocked, has no expired medical supplies, has a sufficiently charged battery, among others) is important to ensure the emergency medical treatment and guidance apparatus is ready for use at all times and increase the confidence of the lay rescuers in performing the medical treatment.

Similarly, a defibrillator, which is useful for lay rescuers to treat cardiac arrest victims, may have functional issues, for example, have a dead battery, include expired electrodes, or have technical problems (e.g., fail a self-evaluation test perhaps because of issues delivering an electrical shock). Ensuring the defibrillator is ready for use at all times is also important because defibrillation as early as possible is a critical component in treating cardiac arrest.

In this context, devices described herein (e.g., an emergency medical treatment and guidance apparatus or a defibrillator) that are ready may refer to devices that are fully functional (e.g., the functions of the device operate comparably to a brand new device) and fully stocked (e.g., having an inventory comparable to a brand new device). In some examples, a ready device includes non-expired medical items such as pharmaceutics, non-expired components such as electrodes, and/or a sufficiently charged battery (e.g., greater than 80% charged). In this way, the device is “ready” when the device is ready to be used to treat all medical emergencies as originally configured.

In embodiments of the present disclosure, the term “readiness” may represent whether or not a device is “ready.” For example, a deployment system determines the readiness of both the emergency medical treatment and guidance apparatuses and defibrillators within the deployment station and provides an indication of the readiness (e.g., transmit a signal to a separate computing device to communicate and/or provide an update as to whether the device is ready or needs inspection) via a readiness indicator. In this way, the readiness indicator communicates the readiness of the device to the lay rescuer as an indication that the device will be ready during the medical treatment as intended.

As discussed herein, the term “needs inspection” may be used to represent a device that is not 100% ready and is in need of attention and/or servicing. For example, a device with at least one medical item missing (e.g., a tourniquet is missing) is in need of attention by an inspector who can replenish the device. However, a device that “needs inspection” may, of course, be used to treat a medical emergency with the caveat that something may be amiss. In this way, a readiness indication that a device “needs inspection” is merely a way to communicate that the device may not be fully functional in every respect (e.g., malfunctioning hardware such as a failed self-diagnostic evaluation, low battery [e.g., less than 20% charge]) and may have missing medical items (e.g., a missing tourniquet) and/or missing components (e.g., missing electrodes for defibrillation). Therefore, in some examples, it is incumbent on the inspector to “attend” to the devices and review whether the “needs inspection” indication still allows the devices to be suitable for use in at least some medical emergencies (e.g., usable although some medical items are missing).

In some examples, when the device is determined to “need inspection,” it may be quantified into levels of severity, for instance: Level 1—“Ready to use,” Level 2—“Needs inspection: safe to use,” Level 3—“Needs inspection: Items missing but safe to use,” and Level 4—“Needs inspection: Unsafe to use.” Some examples of Level 2 where the device needs inspection yet is safe to use may be a partially depleted battery, e.g. >50% that should be replaced but has enough charge to complete a rescue, or some item(s) with expired expiration dates less than 6 months out of date but still functional such as bandages, medication, etc. Some examples of Level 4 where a device may be unsafe for use may be a defibrillator with a depleted battery that is unable to deliver shocks, or more than some predetermined threshold of total number of particular items missing from kit or having expiration dates out of date by more than some predetermined threshold number of days. In other examples, the device may simply provide an indication that it is “ready to use” or that it “needs inspection,” without additional information as to whether the device is safe for use.

Deployment stations monitor the readiness of each emergency medical treatment and guidance apparatus and each defibrillator contained within the deployment station and communicate this readiness to a database as part of a networked readiness management system that manages the overall readiness of each deployment station and the readiness of each emergency medical treatment and guidance apparatus and each defibrillator. The readiness management system also manages the inventory of all devices (e.g., emergency medical treatment and guidance apparatuses, defibrillators, among others), medical supplies within the devices (e.g., tourniquets, pharmaceuticals, among others), hardware of the devices (e.g., batteries, tablets, electrodes, among others), and partial inventory when applicable (e.g., low pharmaceuticals, partially filled bottle of painkillers, among others).

Determining the inventory and readiness may be challenging especially in attempting to increase efficiency of the inspectors (e.g., the personnel whom attend to stocking and serving of the devices within the readiness management system). In this way, cameras, microphones, weight scales, and/or RF communications are used to communicate between each emergency medical treatment and guidance apparatus, each defibrillator, and the deployment station that contains them. For example, when a user returns an emergency medical treatment and guidance apparatus to a deployment station, the RFID tags of the emergency medical treatment and guidance apparatus (and/or each medical item within the emergency medical treatment and guidance apparatus) are read by the deployment station to determine which medical supplies are missing.

The readiness management system also communicates with user devices (e.g., both the devices of the lay rescuers and the inspectors) to provide information about the location, readiness, and inventory of the deployment stations. This communication with user devices also enables the interactive guidance for assisting the lay person in determining which medical devices/items are needed to administer the medical treatment and providing guidance to perform the medical treatment, as described above. In examples of inspector devices, the readiness management system transmits information (e.g., in the form of notifications) to the inspectors' user devices to alert the inspectors of deployment stations that contain emergency medical treatment and guidance apparatuses and/or defibrillators that require inspection (e.g., attention and/or servicing) (e.g., at least some restocking is needed, charging of batteries is needed, expired pharmaceuticals/electrodes, hardware problems, among others). The user devices are also able to interact with the database (e.g., via an application) to retrieve/update/add inventory and/or readiness information with the database.

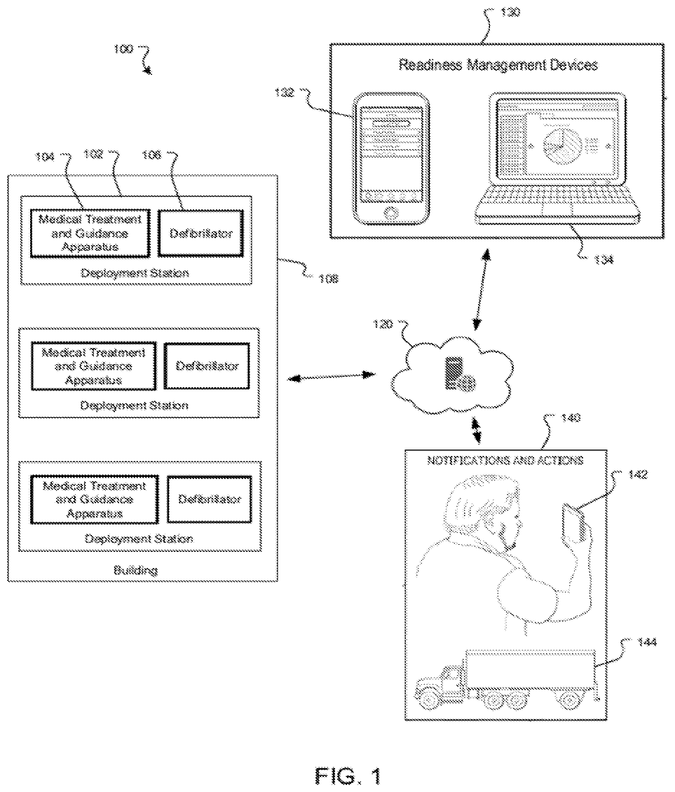

FIG. 1 shows a readiness management system 100 of deployment stations 102 with portable emergency medical treatment and guidance apparatuses 104 and portable defibrillators 106. In some examples, the deployment stations 102 are contained within a building 108 but in other examples, they are distributed among several buildings. In some examples, the building 108 is part of a pre-hospital setting (e.g., an office building, a school, an airport, among others).

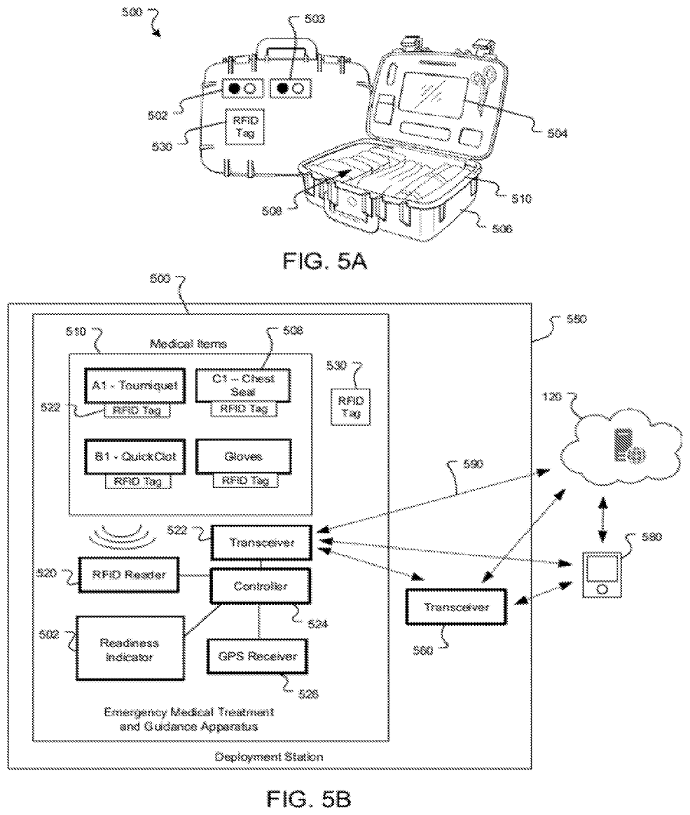

In some embodiments, emergency medical treatment and guidance apparatuses 104 include a plurality of medical supplies (tourniquets, trauma dressing, among others) that can be used to provide medical assistance to a patient during a medical emergency. Further details regarding emergency medical treatment and guidance apparatuses 104 are described with reference to FIGS. 5A, 5B, 6, 7A, and 7B below. Examples of such emergency medical treatment and guidance apparatuses 104 are the ZOLL Mobilize Public Access Utility Kit and the ZOLL Mobilize Public Access Compact Rescue System described in U.S. Application No. 63/123,997, entitled “Inventory Management Of Portable Medical Treatment And Guidance Apparatuses” filed Dec. 10, 2020, and International Application No. PCT/US21/62591 entitled “Inventory Management Of Portable Medical Treatment And Guidance Apparatuses” filed Dec. 9, 2021, the disclosures of which are each hereby incorporated by reference in its entirety.

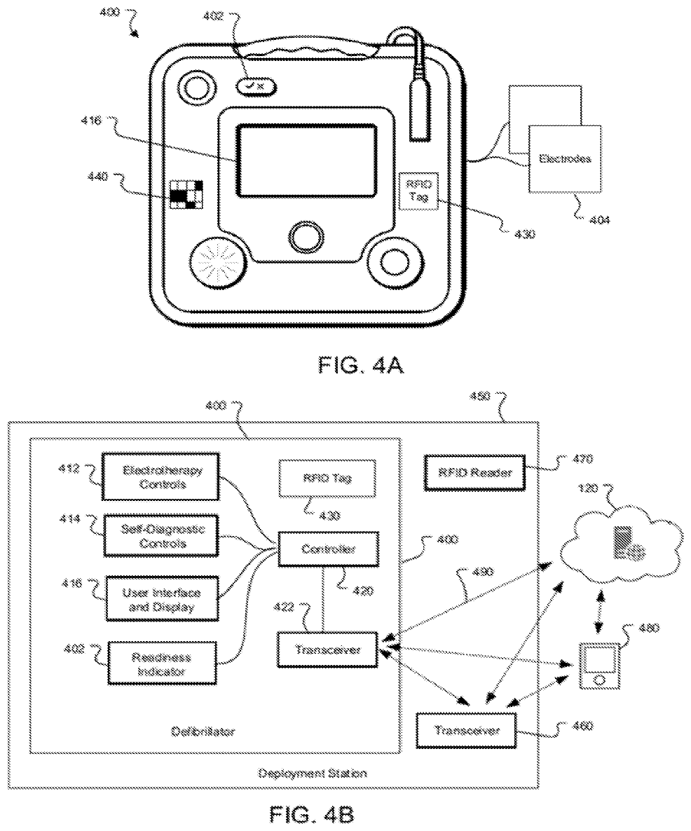

The defibrillators 106 provide medical assistance to a patient during a medical emergency. In some examples, the defibrillator 106 is (or includes) an automated external defibrillator (AED) that is publicly accessible and designed for those who have little to no medical experience to enable the user to administer electrotherapy to a patient according to a resuscitation protocol. In this scenario, the defibrillator 106 includes defibrillation electrodes, shock delivery control, and other defibrillator controls. Further details regarding the defibrillators 106 are described with reference to FIGS. 4A and 4B below.

The deployment stations 102 include computer electronics that allow the deployment stations 102 to communicate with a database hosted by a server 120. The server 120 and the database are part of the readiness management system 100. For example, the deployment stations 102 transmits inventory information, readiness information, and/or usage information of the emergency medical treatment and guidance apparatuses 104 and/or defibrillators 106 within the deployment stations 102 to the server 120 for storage in the database.

As part of the readiness management system 100, the server 120 communicates with readiness management devices 130 such as mobile devices 132 (e.g., a smartphone, a tablet, a smartwatch, among others) and/or a computer 134 (e.g., a laptop, a desktop, among others). The readiness management devices 130 query the information stored in the database hosted by the server 120 to retrieve the inventory information, readiness information, and/or usage information for each emergency medical treatment and guidance apparatus 104 and/or each defibrillator 106 within each deployment station 102 within the readiness management system 100. The readiness management devices 130 can update and/or add inventory information, readiness information, and/or usage information as necessary for each emergency medical treatment and guidance apparatus 104 and/or each defibrillator 106 within each deployment station 102 within the readiness management system 100. For example, the readiness management devices 130 can query the database to determine when at least one of the emergency medical treatment and guidance apparatuses 104 and/or at least one of the defibrillators 106 within the deployment stations 102 requires inspection.

In some embodiments, the readiness management system 100 (e.g., generally the server 120) queries the inventory information, readiness information, and/or usage information of the emergency medical treatment and guidance apparatuses 104 and/or defibrillators 106 contained within each deployment station 102 within the database. The readiness management system 100 executes notifications and actions 140 based on the queried inventory information, readiness information, and/or usage information of the database. For example, the readiness management system 100 can present a notification on an inspector's smartphone 142 to attend to a particular deployment station 102 because at least one device requires inspection. In some examples, the readiness management system 100 submits a request to restock a particular deployment station 102. In this scenario, the request can initiate an order to automatically purchase the missing medical supplies or defective components (e.g., expired electrodes), which may then be delivered, e.g., via a truck 144.

For example, the readiness management system 100 can determine that a battery of a particular emergency medical treatment and guidance apparatus 104 and/or a particular defibrillator 106 is low (e.g., via a connection with electrical circuitry of the battery) and can transmit a notification to the inspector to recharge the battery. The notification can include location information of the deployment station 102 (e.g., via GPS or via manually entered (e.g., “second floor”)). The notification can include information about the particular emergency medical treatment and guidance apparatus 104 and/or the particular defibrillator 106. For example, the readiness management system 100 can determine that a particular medical item of a particular emergency medical treatment and guidance apparatus 104 has been used (e.g., by inferring use from a status report) and can initiate an order to purchase more of the used medical supplies (e.g., by generating a record that may then be used to notify a manager to order or otherwise check to see if the item(s) need replacement, by placing the item(s) on a list for future ordering, amongst other methods).

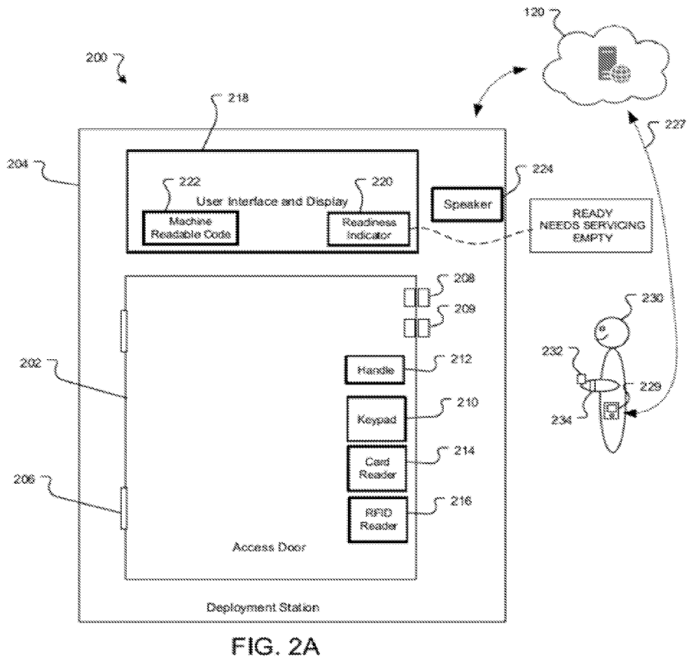

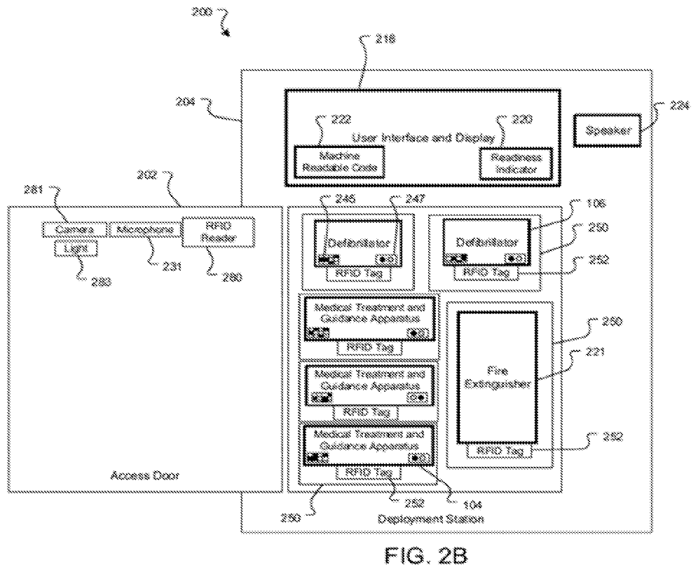

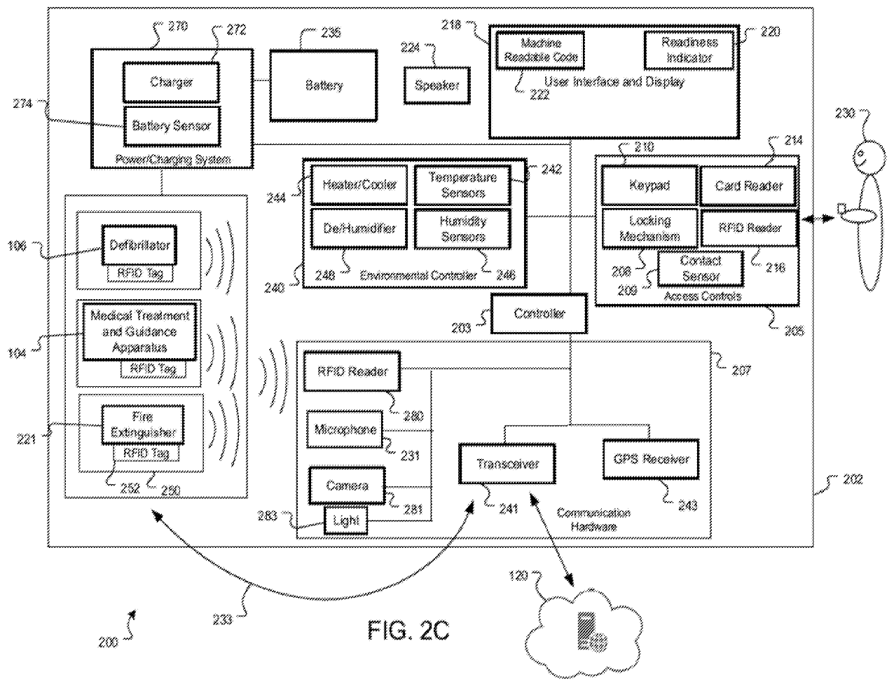

FIGS. 2A and 2B show a deployment station 200 as part of a readiness management system. In some embodiments, the deployment station 200 is the same as, or similar to, deployment station 102 described with reference to FIG. 1. The deployment station 200 includes a housing 204. In some examples, the housing 204 is mounted to a wall of a building using mounting hardware (not shown), but in other examples the housing 204 simply rests on a floor of a building. The housing 204 includes an access door 202 (shown in a closed configuration in FIG. 2A). The access door 202 is pivotably connected to the housing 204 (e.g., via one or more mechanical hinges 206) such that it can move between an open and closed configuration.

In some embodiments, the access door 202 includes a contact sensor 209 to monitor when the access door 202 is in the open vs. closed position. For example, the contact sensor 209 can be a toggle switch that sends a contact signal to a controller 203 (shown in FIG. 2C) of the deployment station 200 when the access door 202 is open. In some examples, the deployment station 200 transmits a signal to a mobile device of an inspector (e.g., via a transceiver 241 of the deployment station 200 shown in FIG. 2C and over the readiness management system 100) when the access door 202 changes from the closed position to the open position. In some examples, the deployment station 200 transmits a signal to the server 120 of the readiness management system 100 for updating the database to include information that the access door 202 changed from the closed position to the open position at a particular date and time.

In some embodiments, the access door 202 includes an electrically controlled locking mechanism 208 (e.g., a magnetic lock) so that the controller 203 can magnetically lock and unlock the access door 202 based on a user's credentials. One of the purposes of the locking mechanism 208 is to reduce theft of the contents of the deployment station 200. For example, a defibrillator is expensive which could incentivize someone to steal the defibrillator for monetary gain. When this is probable, the locking mechanism 208 can be enabled (e.g., by a device setting) control access. In some embodiments, the deployment station 200 maintains an access record of whom is accessing the contents of the deployment station 200 (e.g., the access record can be transmitted to the server 120 from the deployment station 200 and stored in the database).

For example, the deployment station 200 can remain in a locked configuration until the deployment station 200 grants a user 230 access to the deployment station 200 (e.g., via passcode, keycard, RFID tag, mobile communication device such as a mobile phone or tablet, amongst others). For example, the locking mechanism 208 can be unlocked when a user's identification, provided via an access card, via an RFID tag, or via a user device as a passcode, a biometric identification, etc., is verified with a list of authorized users stored in the database hosted by the server 120. However, in some embodiments, the locking mechanism 208 is disabled and instead the access door 202 is openable without user identification.

In some examples, the deployment station 200 may be internet-connected to a server that receives EMS dispatch information and, based on the proximity of the deployment station 200 to the location of the emergency event, the locking mechanism 208 may be disabled remotely by the server and instead the access door 202 is openable without the need for user identification. Alternatively, the user may have previously loaded a deployment station application onto their smartphone or other mobile communications device 229 that grants the user access to the deployment station 200 via Bluetooth or other wireless communication with the deployment station 200. If the user has not yet downloaded the deployment station application upon arrival at the deployment station 200, the application may be easily downloaded by the user by pointing the camera of the mobile communication device at a QR-code or other visually-encoded machine readable information on the deployment station 200, e.g., machine readable code 222 on the deployment station 200. For example, as discussed further below, the machine readable code 222 can encode a hyperlink such that, when imaged by the camera, the hyperlink directs the mobile communication device 229 to automatically download the application or to a web page or app store where the application can be manually downloaded. Even though the user has not been verified by the readiness management system that includes the deployment station 200, e.g., by the application having been previously downloaded and the user's identification stored in the database of the server 120, the deployment station application may have an access override function in some examples, allowing the user to gain access to the deployment station 200 contents. In some examples, initiating the override function may cause geolocation and/or phone number services to be accessed on the mobile communication device 229 by the application so that if the user has failed to return some of the deployment station 200 contents after the fact or has intentionally stolen some of the contents, then the user may be contacted for retrieval of the equipment, or the system may make a record that the particular user may still be in possession of the contents.

In some examples, the user's identification is represented by a passcode, an access card, or an RFID tag of the user. For example, in some cases, the deployment station 200 unlocks the access door 202 when the user 230 enters a passcode (e.g., an alphanumeric passcode) on a keypad 210 of the deployment station 200, e.g., on the access door 202. In this scenario, the deployment station 200 (e.g., the controller 203 thereof) compares the passcode with a list of authorized passcodes (e.g., stored in the database hosted by the server 120 accessible by the deployment station 200), determines that the passcode matches one of the authorized passcodes, and unlocks the locking mechanism 208. Subsequently, the user 230 can open the access door 202 (e.g., by pulling a handle 212 mounted to the access door 202). In this way, the access door 202 prevents access to the contents of the deployment station 200 (e.g., emergency medical treatment and guidance apparatuses 104 and defibrillators 106 (as shown in FIG. 2B)) when in a locked configuration and allows access (or at least does not prevent access) in an unlocked configuration.

As shown in FIG. 2A, in some embodiments, the access door 202 includes a card reader 214 that reads a card 232 of the user 230 when the user inserts the card 232 into the card reader 214. In some examples, the card reader 214 includes a magnetic reader that reads a magnetic stripe of the card 232 when the user 230 swipes the card 232 through the card reader 214. In other examples, the card reader 214 includes a reader that reads an embedded microchip of the card 232. In this scenario, the card reader 214 reads the information stored on the card 232. When the deployment station 200 determines that the information of the card 232 matches information associated with one of the authorized cards (e.g., by comparing the information of the card 232 with information in the database hosted by the server 120), the deployment station 200 unlocks the locking mechanism 208.

In some embodiments, the access door 202 includes an RFID reader 216 that reads an RFID tag 234 of the user 230 (e.g. an RFID tag embedded in an access card or embedded in an amulet of a necklace or bracelet). The RFID reader 216 reads the information stored on the RFID tag 234 of the user 230. When the deployment station 200 determines that the information of the RFID tag 234 matches information associated with one of the authorized RFID tags (e.g., by comparing the information of the RFID tag 234 with information in the database hosted by the server 120), the deployment station 200 unlocks the locking mechanism 208.

In some embodiments, the deployment station 200 includes a user interface and display 218. In some examples, the user interface and display 218 is part of a removable tablet electronic device mounted to the housing 204. In some examples, the user interface and display 218 is a touch screen display mounted to the housing 204 for providing an interactive query to the user 230. For example, the user interface and display 218 can display information about the deployment station 200 (e.g., system settings, network settings (e.g., connected devices within the deployment station 200), temperature/humidity control settings, battery levels, access records, amongst others). The user interface and display 218 can also display inventory information and/or the readiness of the contents within the deployment station 200 (e.g., emergency medical treatment and guidance apparatuses 104, defibrillators 106, and, in some examples, fire extinguishers).

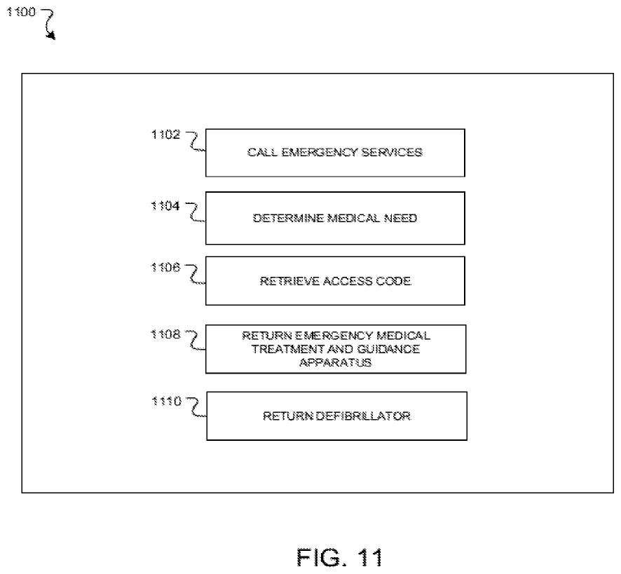

An example scenario is as follows. The user 230 approaches the deployment station 200 and presses the user interface and display 218 to wake up the user interface and display 218 from a sleeping state. The user interface and display 218 presents a notification that the deployment station 200 is locked and requests authorization credentials to unlock the deployment station 200. The user 230 then enters a passcode on the keypad 210, the passcode is verified by the deployment station 200, and then if verified the deployment station 200 unlocks the locking mechanism 208 so the access door 202 can be opened by the user 230. In other examples, the user 230 is an inspector who enters a passcode on the keypad 210 that is unique to inspectors. The deployment station 200 compares the entered passcode with a database of known passcodes for inspectors, verifies the passcode, and then if verified unlocks the locking mechanism 208 so the access door 202 can be opened by the user 230. In this scenario, however, the deployment station 200 is configured to allow full control of all settings, inventory levels, and readiness information of the deployment station 200 via the user interface and display 218. Further aspects of the user interface and display 218 are described with reference to FIG. 11.

As shown in FIG. 2A, the deployment station 200 includes a readiness indicator 220 (e.g., an LED, a symbol, an image, text, amongst others) for indicating a readiness (e.g., ready or needs inspection) of the contents within the deployment station 200 (e.g., the emergency medical treatment and guidance apparatus 104 and/or at least one defibrillator 106 as shown in FIG. 2B). As noted above, in some scenarios, a “ready” indication means that the device is fully stocked and, when applicable, has a sufficiently charged battery (e.g., above 80% charge). Conversely, a determination that the device “needs inspection” indicates that the device requires inspection. In some scenarios, “needs inspection” provides further indication that the device is missing medical supplies (e.g., needs replenishment) and/or includes a low battery (e.g., the battery needs to be charged). In some embodiments, and as discussed further herein, the readiness indicator 220 may provide an indication if a device “needs inspection,” yet is still safe to use, or if a device “needs inspection,” and is unsafe or otherwise unfit for use. Alternatively, the readiness indicator 220 may provide an indication if a device is “ready for use” or “needs inspection,” without further information about whether it is fit for use.

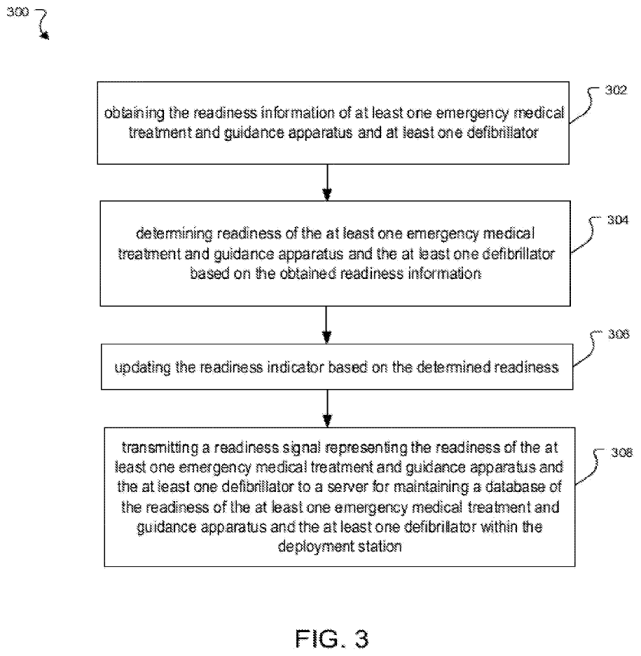

Generally, the readiness indicator 220 is visible by the user 230 so the user 230 can glance at readiness indicator 220 to determine whether the deployment station 200 is ready (e.g., each emergency medical treatment and guidance apparatus is present and fully stocked, each defibrillator is present and functioning properly, amongst others). In the example shown, the readiness indicator 220 is provided on the user interface and display 218 (e.g., the readiness indicator 220 represents one or more pixels of the display 218). The readiness indicator 220 being provided on the user interface and display 218 allows the readiness indicator 220 to be visible to the user 230 regardless of whether the access door 202 is open or closed. In other examples, the readiness indicator 220 is located on an exterior surface of the housing 204 of the deployment station 200. The readiness indicator 220 being provided on the exterior surface of the housing 204 allows the readiness indicator 220 to be visible to the user 230 regardless of whether the access door 202 is open or closed. Details regarding how readiness is determined is described with reference to FIG. 3 below.

In some embodiments, the readiness indicator 220 includes an image such as a green checkmark symbol presented on the user interface and display 218 when all of the emergency medical treatment and guidance apparatuses 104 and all of the defibrillators 106 within the deployment station 200 are ready (e.g., fully stocked, fully charged, amongst others). Similarly, the readiness indicator 220 may include an image such as a yellow caution symbol when at least one of the emergency medical treatment and guidance apparatuses 104 and/or at least one of the defibrillators 106 within the deployment station 200 requires inspection. In some examples, this means that at least one of the emergency medical treatment and guidance apparatuses 104 and/or at least one of the defibrillators 106 require servicing (e.g., missing at least one medical item, low battery, expired pharmaceuticals, amongst others). Furthermore, the readiness indicator 220 may include an image such as a red ‘x’ symbol when a majority of the emergency medical treatment and guidance apparatuses 104 and/or a majority of the defibrillators 106 within the deployment station 200 requires inspection (e.g., the entire deployment station 200 is empty, 51% of the contents of the deployment station 200 need inspection, amongst others).

In general, a “needs inspection” indication represents that at least some of the contents within the deployment station 200 require attention or service, although a remainder of the contents of the deployment station 200 may be usable in an emergency (e.g., ready). In other words, a “needs inspection” indication does not necessarily indicate that devices stored by the deployment station 200 cannot be used. Instead, the intent is to provide an indication to an inspector or user 230 that at least one emergency medical treatment and guidance apparatus 104 and/or at least one defibrillator 106 requires inspection. The readiness indicator 220 may provide further information regarding whether the at least one emergency medical treatment and guidance apparatus 104 and/or at least one defibrillator 106 is safe or otherwise fit for use.

In the examples above, the readiness indicator 220 can be one of two or more states (e.g., a “ready” state, a “needs inspection and is fit to use” state, a “needs inspection and is unfit for use” state; or a “ready” state” and a “needs inspection” state). Such states may apply to the deployment station 200 itself and/or contents within the deployment station 200. As discussed, in some embodiments, the readiness indicator 220 involves more than two states (e.g., 3, 4, 5, etc.) to provide a level of granularity to the indication. For example, a third state can be “empty” to indicate that the deployment station 200 is empty. In some embodiments, the readiness indicator 220 is a progress bar between 0% and 100%. In this scenario, 0% can represent an empty deployment station 200 and/or all of the contents within the deployment station 200 require inspection while 100% can represent a fully stocked deployment station 200 and where contents of the deployment station 200 are fully ready.

In some embodiments, the deployment station 200 includes more than one readiness indicator 220 where each readiness indicator 220 provides different types of status information. For example, the deployment station 200 may include two readiness indicators 220 (e.g., in some examples, both readiness indicators are combined within the readiness indicator 220). In such examples, one readiness indicator 220 is used to indicate readiness of tablet devices (e.g., tablet devices of each of the emergency medical treatment and guidance apparatuses within the deployment station) and/or software applications configured to run on the tablet devices (e.g., whether the software version is up to date), while the other readiness indicator 220 is used to indicate readiness of the medical supplies within the emergency medical treatment and guidance apparatuses 106.

For example, when at least one battery of a tablet device within the deployment station 200 changes from a charged state (e.g., above 50% battery charge) to a low charged state (e.g., below 50% battery charge), one of the readiness indicators 220 changes from a “ready” state to a “needs inspection” state, while the second readiness indicator 220 remains in a “ready” state when the devices within the deployment station 200 are otherwise ready. In this example, the deployment station 200 updates the first readiness indicator 220 based on a portion of the determined readiness relating to a tablet device and/or software application of the emergency medical treatment and guidance apparatuses 106, and the deployment station 200 updates the second readiness indicator 220 based on a remainder portion of the determined readiness. In this way, the first readiness indicator 220 indicates to the user/inspector 230 that a tablet device and/or software application requires attention, while the second readiness indicator 200 indicates to the user/inspector 230 that the devices are otherwise ready except for the issues with the tablet device and/or the software application running on the tablet device.

In some examples, readiness of the software application (or “first application”) is determined by a second software application running in the background on the tablet device (e.g., the second software application periodically opens the first software application, performs a set of functions (e.g., ping the deployment station 200 and/or the server 120) and confirms the first software application performs the set of functions correctly (e.g., waits for a response back from the deployment station 200 and/or the server 120 to acknowledge receipt). In other scenarios, the role is reversed, and the deployment station 200 pings the tablet device to confirm the software application is running correctly. Or, in some cases, the software application may provide an indication that the software requires an update to the latest version and needs sufficient time or a suitable network connection for the update to occur.

In some embodiments, the deployment station 200 includes a machine-readable code 222 (such as a barcode or a QR code). In the example shown, the machine-readable code 222 is provided on the user interface and display 218 (e.g., the machine-readable code 222 represents one or more pixels of the display 218). In general, the machine-readable code 222 is used to provide information (e.g., inventory, readiness, among others) about the deployment station 200 and the contents within the deployment station 200 to the user 230. For example, when the user 230 scans the machine-readable code 222 with a mobile device 229, the mobile device 229 presents readiness information to the user 230 concerning the devices housed by the deployment station 200 and/or the deployment station 200 itself. In this way, the machine-readable code 222 represents the readiness of the emergency medical treatment and guidance apparatuses 104 and/or the defibrillators 106 within the deployment station 200.

While information can be directly encoded into the machine-readable code 222, in some examples it is advantageous to encode a hyperlink such that, when the machine-readable code 222 is imaged by the mobile device 229, the hyperlink directs the mobile device 229 to retrieve detailed information about the inventory and/or readiness of the contents of the deployment station 200. For example, the hyperlink can direct the mobile device 229 to retrieve detailed information from the database of the server 120 (e.g., as shown using communication link 227). The detailed information can include any of the information accessible by the deployment station 200 and the readiness management system 100 in general. For example, the identifying information (e.g., serial numbers, device type), usage information (e.g., last used, for which medical treatment, status report information), expiration information (expiration of pharmaceuticals, expiration of electrodes) of the emergency medical treatment and guidance apparatuses 104 and/or defibrillators 106 within the deployment station 200, as well as system information (e.g., network settings, access information) of the deployment station 200.

For example, the machine-readable code 222 can encode a hyperlink such that, when the machine-readable code 222 is imaged by the mobile device 229, the hyperlink directs the mobile device 229 to retrieve connection information from the database and display information related to which devices are in network communication with the deployment station 200 (e.g., all devices within the readiness management system 100).

In some examples, the machine-readable code 222 encodes a hyperlink such that, when the machine-readable code 222 is imaged by the mobile device 229, the hyperlink directs the mobile device 229 to retrieve contact information of the deployment station 200. For example, mobile device 229 retrieves the information from the database hosted by the server 120 and displays information about who to contact to obtain access to the deployment station 200.

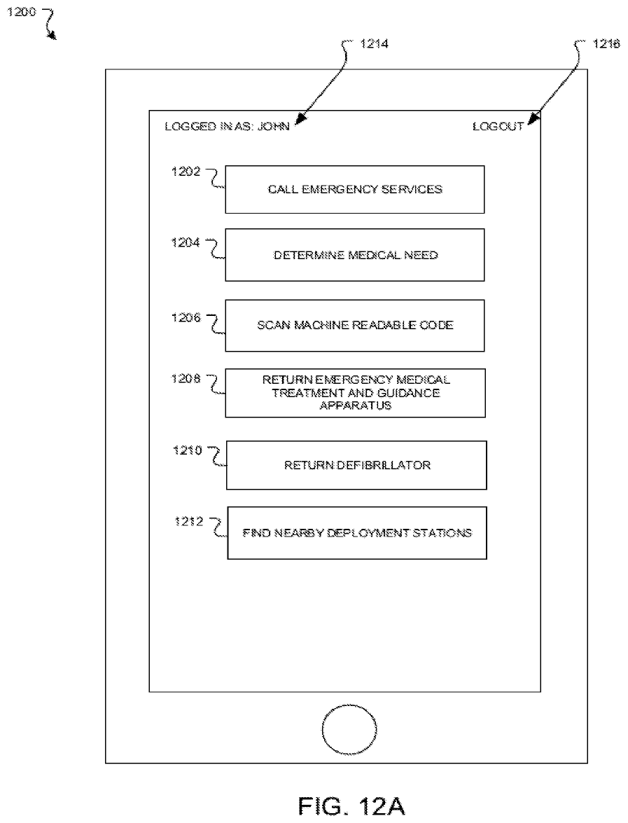

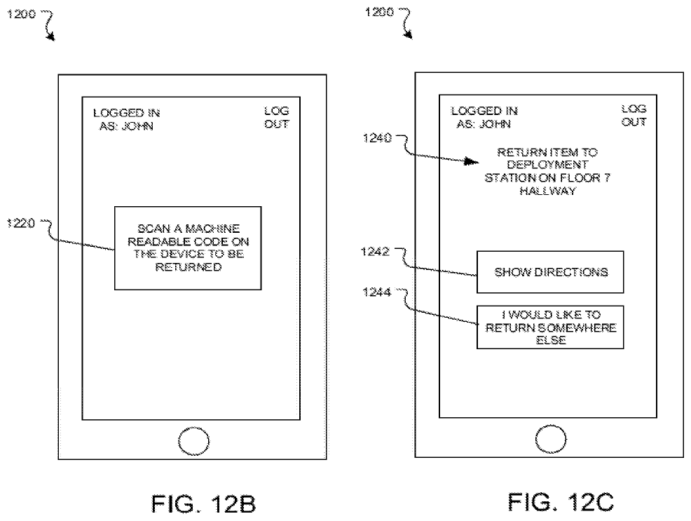

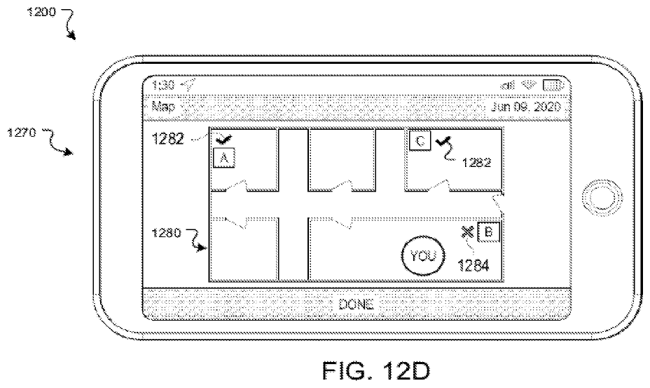

In some examples, the machine-readable code 222 encodes a hyperlink such that, when the machine-readable code 222 is imaged by the mobile device 229, the hyperlink directs the mobile device 229 to retrieve an application for use with the readiness management system 100. For example, the hyperlink directs the mobile device 229 to a download server for downloading an application 1200 described with reference to FIGS. 12A-12D below.

As shown in FIG. 2A, in some embodiments, the housing 204 includes a speaker 224. In some examples, the speaker 224 transmits an audible notification to the user 230 to supplement the interactive query of the user interface and display 218. In some examples, the audible notification is an instruction to the user 230, e.g., “Insert passcode,” or “Insert keycard,” or a response to the user 230, e.g., “EMS notified,” or “Take the defibrillator.”

FIG. 2B shows the deployment station 200 with the access door 202 in an open configuration. For example, the access door 202 opens after the deployment station 200 unlocks the locking mechanism 208 and the user 230 opens the access door 202. The housing 204 of the deployment station 200 includes storage sections 250 for holding at least one emergency medical treatment and guidance apparatus 104, at least one defibrillator 106, and a fire extinguisher 221. The storage sections 250 are located within the housing 204. In the example shown, the deployment station 200 has six storage sections 250, three of which include an emergency medical treatment and guidance apparatus 104, two of which include a defibrillator 106, and one of which includes a fire extinguisher 221.

In some examples, the storage sections 250 that receive the emergency medical treatment and guidance apparatus 104 are first storage sections 250, and the storage sections 250 that receive the defibrillator 106 are second storage sections 250. In some examples, more than one emergency medical treatment and guidance apparatus 104 and/or more than one defibrillator 106 is storable within a single storage section 250 (e.g., the storage section 150 is sufficiently sized). In some examples, an emergency medical treatment and guidance apparatus 104 and a defibrillator 106 is storable within the same storage section 250.

One of the purposes of the storage section 250 is to keep the deployment station 200 organized so that users 230 can access the emergency medical treatment and guidance apparatuses 104, the fire extinguisher 221, and/or the defibrillators 106 as quickly as possible. Furthermore, the user interface and display 218 can instruct the user 230 to take a particular device from within a particular storage section 250. In this scenario, each storage section 250 can be labeled “A1, A2, B1” etc., and the instruction can indicate by label where the particular device is located within the deployment station 200. Aspects related to the deployment station 200 instructing the user 230 to take a particular device within a particular storage section 250 are described with reference to FIG. 11A.

In some examples, each storage section 250 includes a releasable mount (not shown) located within the storage section 250 for releasably mounting the emergency medical treatment and guidance apparatus 104, the fire extinguisher 221, and/or the defibrillator 106 within the storage section 250. In some examples, the releasable mount is a Velcro strap or a latch and hook system. In other examples, the emergency medical treatment and guidance apparatus 104, the fire extinguisher 221, and/or the defibrillator 106 is configured to rest on a bottom surface of the first storage section 250 without a mount (e.g., under the action of Earth's gravity).

In some embodiments, each emergency medical treatment and guidance apparatus 104, each defibrillator 106, and the fire extinguisher 221 (collectively “devices”) includes a respective RFID tag 252. In some examples, the deployment station 200 reads the RFID tags 252 using an RFID reader 280 to determine which devices are present within the deployment station 200. In some embodiments, the RFID reader 280 is mounted on the access door 202 of the deployment station 200. As shown in FIG. 2B, the RFID reader 280 is mounted on the inside of the access door 202, however in other examples, the RFID reader 280 is mounted on or within the housing 204 of the deployment station 200. A RFID reader 280 is capable of reading multiple RFID tags 252 and, therefore, in some examples, only one RFID reader 280 is needed to read all RFID tags 252 within the deployment station 200. However, as explained in the following paragraph, there are situations where more than one RFID reader 280 is beneficial.

For example, when a RFID tag 252 of a respective device is sufficiently close to the RFID reader 280 (e.g., less than 2 feet away from the deployment station 200), the RFID reader 280 receives a signal from the RFID tag 252 and that signal includes information about the particular device. However, as the same device is removed from the deployment station 200, the respective RFID tag 252 becomes further and further away from the RFID reader 280 such that eventually the RFID tag 252 is too far from the RFID reader 280 (e.g., greater than 2 feet away from the deployment station 200) and no signal is received. In this scenario, the deployment station 200 determines that the device associated with the particular the RFID tag 252 is no longer present within the deployment station 200. In some examples, each storage section 250 includes a RFID reader 280 along with electromagnetic shielding such as copper sheeting, nickel or silver-coated carbon fiber or other shielding film, fabric or other shielding material such that the signals received by each of the RFID readers 280 has sufficiently attenuated the signal strength such that the RFID tag 252 is only readable by the RFID reader 280 in that particular storage section 250 the respective device is located in.

In addition to determining the presence of each device within the deployment station 200, identifying information can be transmitted using the RFID information signal. For example, the RFID tags 252 associated with each device broadcast identifying information about the respective device (e.g., serial numbers). In some examples, the identifying information includes the type of device (e.g., whether the device is an emergency medical treatment and guidance apparatus 104, a defibrillator 106, or a fire extinguisher 221). In some examples, the identifying information includes information about the medical supplies and/or components of a particular device (e.g., whether the device includes medical supplies and, if so, what medical supplies and/or components the device includes, e.g., a tourniquet, electrodes, pharmaceuticals, etc.). In some examples, the identifying information includes detailed information about the items (e.g., both medical and/or component/hardware related) of the device (e.g., expiration dates of pharmaceuticals, battery charge levels of batteries, expiration dates of electrodes for administering electrotherapy, expiration date of a medical item, among others). In some examples, the RFID tags 252 associated with electronic devices such as batteries, defibrillators 106, etc. may also include electrical connections to the associated electronic devices. The electrical connection may be an electronic communication interface such as USB, RS232, CAN or other suitable communication interface to allow for updating of information stored on the RFID tags 252. Information to be updated may, in some examples, be battery charge level, expiry information, usage information, defibrillation shocks or other clinical usage data. In some examples, expiration dates and the like are not updated and instead are fixed once programmed into the RFID tags 252 at time of assembly. Further details of the processing and determination of the identifying information is described with reference to FIG. 2C below.

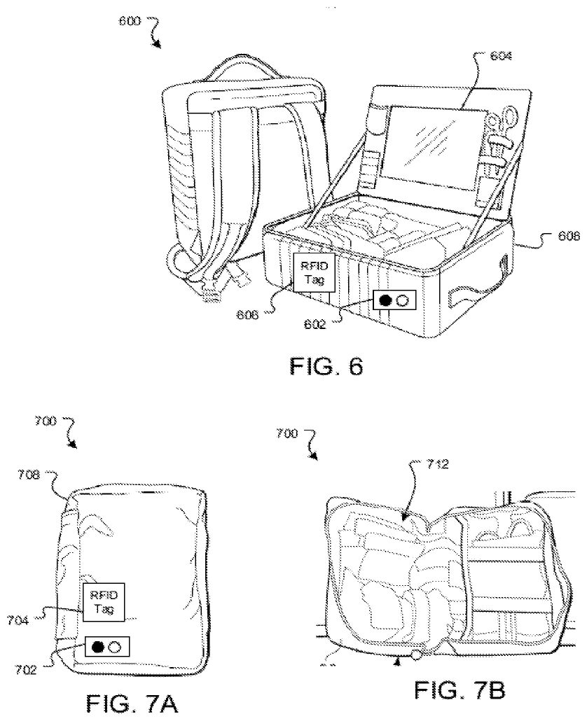

In some embodiments, the deployment station 200 includes a microphone 231. In the example shown, the microphone 231 is mounted to the access door 202 (e.g., on the inside of the access door 202 as shown in FIG. 2B), but in other embodiments, the microphone 231 is mounted to the housing 204. The microphone 231 receives audible signals from at least one speaker of the emergency medical treatment and guidance apparatus 104, the defibrillator 106, and/or the fire extinguisher 221. A purpose of the audible signal is to transfer information from these devices to the deployment station 200. For example, an emergency medical treatment and guidance apparatus 104 that includes a speaker can optionally transmit a first audible signal when ready and a different audible signal when an inspection is needed, or may simply transmit an audible signal when an inspection is needed without an audible signal when the apparatus is ready and able for use. For example, emergency medical treatment and guidance apparatuses 500 and 600, which are described below with reference to FIGS. 5A, 5B, and 6, respectively, include a speaker for transmitting the audible signal.

In some embodiments, the emergency medical treatment and guidance apparatus 104, the defibrillator 106, and/or the fire extinguisher 221 includes circuitry for encoding an audible message about the identifying information and/or the readiness information into the audible signal (e.g., via frequency encoding, amplitude encoding, Morse code encoding, among others) and transmitting the audible signal representing identifying information and/or readiness information via the speaker of the respective emergency medical treatment and guidance apparatus 104, defibrillator 106, and/or fire extinguisher 221. In this scenario, the deployment station 200 receives the audible signal via the microphone 231 and decodes (e.g., using the controller 203 in operative communication with the microphone 231) the audible message encoded in the audible signal.

In some examples the encoding scheme is a frequency encoding scheme where, optionally, a first frequency (e.g., 2 kHz) indicates that a device is ready for use and a second frequency (e.g., 4 kHz) indicates that the device needs inspection. For example, a defibrillator 106 may broadcast a 2 kHz frequency tone periodically (e.g., every two seconds) with a determined tone width (e.g., one second) and a wait time between each periodic tone (e.g., a wait time of 0.5 seconds) to represent that the defibrillator 106 is ready for use, and the defibrillator 106 may broadcast a 4 kHz frequency tone periodically (e.g., every two seconds) with the same tone width (e.g., one second) and the wait time between each periodic tone (e.g., a wait time of 0.5 seconds) to represent that the defibrillator 106 is ready for use but needs inspection. In other examples, the tone width, the wait time, and the amplitude of the tone varies to represent whether the defibrillator 106 is ready for use, needs inspection yet is still ready for use, or needs inspection and is unfit for use. Or, the tone width, the wait time, and the amplitude of the tone may vary to represent whether the defibrillator 106 is ready for use or simply needs inspection. Alternatively, in various embodiments, as discussed herein, the audible signal may be generated only when a device needs inspection. The audible signal may employ an encoding scheme such as a frequency encoding that a signal processor may read so as to determine whether the device needs inspection, and optionally further information, such as whether the device is fit (e.g., safe) for use or is unfit (e.g., unsafe) for use. In various embodiments, a frequency encoding scheme may involve audio frequency-shift keying (AFSK). In some examples, the AFSK may be binary, continuous phase, Gaussian, minimum-shift or combination such as Gaussian minimum shift AFSK. AFSK is a modulation technique by which digital data is represented by changes in the frequency (pitch) of an audio tone, yielding an encoded signal suitable for transmission via radio or telephone. Normally, the transmitted audio alternates between two tones: one, the “mark,” represents a binary one; the other, the “space,” represents a binary zero. AFSK differs from regular frequency-shift keying in performing the modulation at baseband frequencies. By using such an encoding scheme, more detailed information can be transmitted to the deployment station that just whether the device is “Ready” or not.

In some embodiments, the deployment station 200 transmits a request to the server 120 to update the database based on the decoded audible message. For example, after receiving an audible message indicating that a particular device needs inspection, and optionally further whether the device is fit for use or unfit for use, the deployment station 200 transmits a request to the server 120 to update the database to indicate that the particular device needs service and is still fit for use, or needs service and is further unfit for use, or whether the device is no longer “ready” or “fully ready.”

In some embodiments, the deployment station 200 includes a camera 281 and a light 283 mounted on the access door 202 of the deployment station 200. As shown in FIG. 2B, the camera 281 and the light 283 are mounted on the inside of the access door 202, however in other examples, the camera 281 and/or the light 283 are mounted on or inside the housing 204 of the deployment station 200. The camera 281 is capable of imaging multiple emergency medical treatment and guidance apparatuses 104, defibrillators 106, and/or fire extinguishers 221 within the deployment station 200.

In some examples, the camera 281 images a respective individual readiness indicator 247 exposed on an outer surface of each emergency medical treatment and guidance apparatus 104 and each defibrillator 106 to determine readiness information of the respective devices. In some examples, the individual readiness indicator 247 of each device is the same as, or substantially similar to, the readiness indicator 220 of the deployment station 200. In some examples, the individual readiness indicators 247 are a micropower (<0.1 mW) LCD or electrophoretic (e.g., e-Ink) display in electrical communication with a controller of the respective device. The display may be configured to display graphically-encoded digital information, for instance as QR-codes or barcodes, so that multiple bytes of readiness and test result information can be communicated with the deployment station 200, and thereby transmitted to the personnel managing the system by the personnel using, e.g., a mobile device, to scan the graphically-encoded digital information and display the readiness and test result information and/or transmit the readiness and test result information to the server 120. Specific test results for defibrillator energy, charge time, ECG bandwidth, frequency response, battery levels, etc. may be simultaneously displayed within the single QR or bar code. In some examples, multiple frames of the graphically-encoded digital information may be displayed in succession to increase informational content. The deployment station camera 281 may be placed in video mode and the video images processed (e.g., by the controller 203 of the deployment station 200 and/or by the server 120 in communication with the deployment station 200) to determine frame boundaries of the QR or barcode, extract each clean QR or bar code frame, and extract the digital information from the QR or barcode.

In some examples, the individual readiness indicator 247 is dynamically controlled using a controller of the respective device. For example, the respective device's controller can read RFID tags of each medical supply or medical item within the respective device periodically (e.g., minutely, hourly, daily, and/or the like) and update the individual readiness indicator 247 based on the received RFID information. For example, if the respective device's controller determines that at least one RFID tag is missing (e.g., the RFID tag was not read but should have been), then the respective device's controller can cause the individual readiness indicator 247 to display a first indication. In another example, if the respective device's controller determines that all RFID tags are present (e.g., all RFID tags were read), then the respective device's controller can cause the individual readiness indicator 247 to display a second indication. Further details about the individual readiness indicators 247 are described with reference to FIGS. 5A, 5B, 6, 7A, and 7B below and details regarding how readiness is determined is described with reference to FIG. 3 below.

For example, the camera 281 can acquire an image of an individual readiness indicator 247 of an emergency medical treatment and guidance apparatus 104 and the camera 281 or the controller 203 in operative communication with the camera 281 can determine the readiness from the image of the individual readiness indicator (e.g., using image processing). For example, the deployment station 200 can identify, for example, a green LED vs. a yellow LED based on the color information of pixels in the image. In this scenario, the green LED would indicate that the emergency medical treatment and guidance apparatus 104 is ready and the yellow LED would indicate that the emergency medical treatment and guidance apparatus 104 needs inspection. In some examples, a single light source can digitally encode the detailed information via such encoding schemes such as run-length limited (RLL) encoding, more particularly differential Manchester encoding (½ RLL) with clock bits. Alternatively the information for the single LED may be encoded via Infra-Red Data Association (IrDA) data formats. In some examples, two or more light emitters such as LEDs with distinct spectral characteristics can be used to digitally encode information by assigning the distinct spectral pattern for each LED to a bit value, e.g., yellow=1, green=2, blue=4, red=8, and then storing the colors as a hexadecimal bit pattern, taking values 0-15. By adding a second intensity level to each LED and then encoding, values from 0-255 may be encoded. The camera 281 can be a high speed camera to decode the LED patterns with bit rates of up to approximately one-half the frame rate of the camera. Current camera technology has frame rates of up to 1000 fps, therefore current data transfer rates by this method are approximately 500 Bps. In other examples, four or more LEDs may be configured in a grid, e.g. 2×2, 4×4, etc., so that words may be encoded and data transfer rates can be doubled to, for instance, 1 KBps in the example above. In other examples, the deployment station 200 (e.g., the controller 203 thereof) can be trained (e.g., in a machine learning sense) to identify text or images of the individual readiness indicators 247 (e.g., a checkmark vs. an ‘x’). In this scenario, the deployment station 200 (e.g., the controller 203 thereof) performs a machine learning analysis on the image to determine whether the emergency medical treatment and guidance apparatus 104 is ready or needs inspection.

In addition to (or in lieu of) imaging the individual readiness indicators 247, in some examples, the camera 281 images a respective machine-readable code 245 of each emergency medical treatment and guidance apparatus 104 and/or defibrillator 106. The machine-readable code 245 encodes a code that is readable by the camera 281 of the deployment station 200 in a similar way as the machine-readable code 222 of the deployment station 200 encodes a code readable by the mobile device 229 of the user 230 as described with reference to FIG. 2A above. For example, identifying information can be directly encoded in the machine-readable code 245 or a hyperlink can be encoded in the machine-readable code 245 that directs the camera 281 (or the controller 203) to the database of the server 120 to retrieve any information stored in the database (e.g., identifying and/or readiness information). For example, the respective device can transmit readiness information directly to the server 120 to be stored in the database. The deployment station 200 can subsequently retrieve this readiness information using the hyperlink as an address within the database.

In some embodiments, the machine-readable code 245 may be a sticker adhered to the housing of the respective device. In this scenario, the machine-readable code 245 is a static code that does not change over time. In other embodiments, the machine-readable code 245 is a low-cost e-ink display that is in electrical communication with a controller of the respective device. In this scenario, the machine-readable code 245 is a dynamic code that changes over time such that readiness information can be encoded in the machine-readable code 245. For example, the machine-readable code 245 can be a display with pixels that are enabled and disabled by the respective device's controller based on the readiness information. In this example, if the machine-readable code 245 is a display with 12 pixels arranged in a 3×4 rectangular grid, as shown in the example of FIG. 2B, each arrangement of pixels represents a unique message. FIG. 2B shows different machine-readable code 245 for each of the emergency medical treatment and guidance apparatus 104 and defibrillators 106. In this example, a machine-readable code 245 with a 12 pixel display can represent a 12 bit display with 4096 unique combinations; however any number of pixels and bits can be used as contemplated with modern display technology (e.g., 100-1000 pixels per inch).

In this example, the machine-readable code 245 can be dynamically controlled using the controller of the respective device. For example, as in the example described above with reference to the readiness indicator 247, the respective device's controller can read RFID tags of each medical supply or medical item within the respective device periodically (e.g., minutely, hourly, daily, and/or the like) and update the machine-readable code 245 based on the received RFID information. For example, if the respective device's controller determines that at least one RFID tag is missing (e.g., the RFID tag was not read by an RFID reader of the device, but should have been), then the respective device's controller can cause the machine-readable code 245 to display a first indication. In another example, if the respective device's controller determines that all RFID tags are present (e.g., all RFID tags were read), then the respective device's controller can cause the machine-readable code 245 to display a second indication. In this way, the machine-readable code 245 can be similar to the readiness indicator 247.

In some examples, the controller of the respective device can periodically update the pixel pattern of the machine-readable code 245 based on which RFID tags are detected within the device. For example, the respective device's controller can illuminate a symmetric pattern of pixels to represent that all medical supplies are present, while an asymmetric pattern of pixels can represent that at least one medical supply is missing. A further level of granularity is possible by encoding which medical supply is missing into the pixels pattern. For example, a first pattern can indicate that a medical supply is missing while a second pattern can indicate that a battery is in need of charge. Again, a determination regarding which medical supplies are missing can be determined based on the RFID tags that were detected by an RFID reader within the device. Another level of granularity is possible in cases where the machine-readable code 245 has a sufficient number of pixels to represent each medical supply with the device. For example, a first pattern can indicate that a tourniquet is missing while a second pattern can indicate that a roll of gauze is missing. Further details regarding how readiness is determined and updated is described with reference to FIG. 3 below.

In some examples, processing of the machine-readable code 245 is the same as, or substantially similar to, the processing of the image of the individual readiness indicators 247 as described above (e.g., using image processing sometimes in combination with a machine learning system). In this way, the deployment station 200 determines identifying information of the respective device based on the image. And in some examples with dynamic or hyperlinked machine-readable codes 245, the deployment station 200 determines readiness information (and generally any information stored within the database) of the respective device based on the image.

In some embodiments, the camera 281 images the housing of the emergency medical treatment and guidance apparatus 104 and/or the housing of the defibrillator 106. In this scenario, the deployment station 200 (e.g., the camera 281 and/or the controller 203) performs image processing (e.g., edge detection, color information, shape/size analysis) to determine the identity of the imaged device. For example, the deployment station 200 queries the database of the server 120 to retrieve expected edges, colors, shapes, and sizes for each variant of the emergency medical treatment and guidance apparatuses 104 (e.g., emergency medical treatment and guidance apparatus 500 vs. emergency medical treatment and guidance apparatus 600 vs. emergency medical treatment and guidance apparatus 700 as described with reference to FIGS. 5, 6, and 7A and 7B, respectively). Similarly, the deployment station 200 queries the database to retrieve expected edges, colors, shapes, and sizes for each variant of defibrillators 106 (e.g., different makes/models such as defibrillator 400 described with reference to FIGS. 4A and 4B). In some examples, a machine learning system of the camera 281 and/or the controller 203 is trained using images of each housing and the machine learning system is used to determine the identity of the using based on the trained images.

In some examples, the light 283 (e.g., an LED flash) illuminates individual readiness indicators 247 and/or the machine-readable codes 245 of each emergency medical treatment and guidance apparatus 104 and/or defibrillator 106. In some examples, the light 283 illuminates the individual readiness indicators 247 and/or the machine-readable codes 245 when the camera 281 is acquiring the image.

FIG. 2C shows a wiring diagram schematic of the deployment station 200. The deployment station 200 includes a controller 203 (e.g., at least one processor and memory), a power and charging system 270, an environmental controller 240, access controls 205, and communication hardware 207. In general, the controller 203 is mechanically coupled to the housing 204 and is in communication (e.g., either wired or wireless) with all electrical devices within the deployment station 200.

In some embodiments, the power and charging system 270 includes a charger 272 for charging batteries of the emergency medical treatment and guidance apparatuses 104 and/or the defibrillators 106. In some embodiments, the charging system 270 is mounted to the housing 204. In examples where at least one of the emergency medical treatment and guidance apparatuses 104 includes a tablet device with a built-in battery (e.g., emergency medical treatment and guidance apparatuses 500 and 600 as shown in FIGS. 5A, and 6, respectively), the charger 272 can include a physical cable that plugs into the charging port of the tablet device for charging the built-in battery and supplies electrical charge from a battery 235 of the deployment station 200 or from building power that provides electrical charge to the deployment station 200. In some examples, the battery 235 of the deployment station 200 provides electrical charge to all the electrical components of the deployment station 200. In other examples, building power provides electrical charge to all the electrical components of the deployment station 200. In this way, the deployment station 200 can be installed in areas without building power access or where power is occasionally interrupted.

In some examples, the charger 272 charges the built-in battery of the emergency medical treatment and guidance apparatus 104 wirelessly (e.g., using induction charging). In examples where the defibrillator 106 includes a built-in battery, the charger 272 can plug into the charging port of the defibrillator 106 for charging the built-in battery. In some examples, the charger 272 charges the built-in battery of the defibrillator 106 wirelessly.

In some embodiments, the charging system 270 includes a battery sensor 274 that determines a status of a built-in battery of the emergency medical treatment and guidance apparatus 104 and/or the defibrillator 106 (e.g., battery charge, battery health, battery usage). For example, when the charger 272 is connected to a built-in battery of the emergency medical treatment and guidance apparatus 104 and/or the defibrillator 106 within the deployment station 200 (e.g., using either with a wired or wireless connection), the battery sensor 274 receives a signal from the built-in battery of the emergency medical treatment and guidance apparatus 104 and/or the defibrillator 106 indicating the remaining charge of the built-in battery and whether the built-in battery is healthy. In some examples, the deployment station 200 receives the battery status via a Bluetooth or other near-field connection. In some examples, the charger 272 controls the charging of the built-in batteries of the emergency medical treatment and guidance apparatus 104 and/or the defibrillator 106 based on the respective battery status, e.g., based on the remaining charge of the built-in battery and/or the health of the built-in battery.

In some embodiments, the environmental controller 240 includes temperature sensors 242, a heater/cooler 244, humidity sensors 246, and a humidifier/dehumidifier 248. In some embodiments, the environmental controller 240 is mounted to the housing 204. In some examples, the environmental controller 240 controls the heater/cooler 244 based on a measured temperature from the temperature sensors 242 to maintain a pre-determined target temperature (e.g., 70 degrees Fahrenheit) or a pre-determined target temperature range (e.g., between 65-75 degrees Fahrenheit) of an interior volume of the housing 204. In some examples, the pre-determined target temperature is based on a temperature sensitivity of the tablet devices within the emergency medical treatment and guidance apparatuses 104 within the deployment station 200. In this example, the pre-determined temperature range can be between −4 and 140 degrees Fahrenheit. In particular, the pre-determined temperature range can be between 32 and 104 degrees Fahrenheit.

In some examples, the environmental controller 240 controls the temperature of the storage sections 250 (e.g., by turning heater/cooler 244 on/off as needed) based on a pre-determined target temperature (or a pre-determined target temperature range) of one or more pharmaceuticals within emergency medical treatment and guidance apparatuses 104 stored within the storage sections 250. For example, the environmental controller 240 can control the heater/cooler 244 to maintain a pre-determined target temperature of 72 degrees Fahrenheit or a pre-determined target temperature range between 68-77 degrees Fahrenheit of the one or more pharmaceuticals within the emergency medical treatment and guidance apparatuses 104. In this scenario, if the deployment station 200 determines that Children's Benadryl is located within the deployment station 200 (e.g., via the RFID tag information), the deployment station 200 (e.g., the controller 203 thereof or the environmental controller 240 using RFID information received from the controller 203) sets the pre-determined target temperature range to be between 68-77 degrees Fahrenheit. In other scenarios, the deployment station 200 sets the pre-determined target temperature range to a different temperature range.

In other examples, the environmental controller 240 controls the temperature of the storage sections 250 (e.g., by turning heater/cooler 244 on/off as needed) based on a pre-determined target temperature (or a pre-determined target temperature range) of at least one electrode of defibrillators 106 stored within the storage sections 250, similar to that discussed above regarding temperature control based on a pre-determined target temperature (or a pre-determined target temperature range) of one or more pharmaceuticals.

In some examples, the environmental controller 240 controls the humidifier/dehumidifier 248 based on a measured humidity from the humidity sensors 246 to maintain a pre-determined target humidity (e.g., 40% humidity) or a pre-determined target humidity range (e.g., between 0-60% humidity) of an interior volume of the housing 204. In some examples, the environmental controller 240 controls the humidity of the storage sections 250 (e.g., by turning the humidifier/dehumidifier 248 on/off as needed) based on a pre-determined target humidity (or a pre-determined target humidity range) of one or more pharmaceuticals within the emergency medical treatment and guidance apparatuses 104 within the storage sections 250. In other examples, the environmental controller 240 controls the humidity of the storage sections 250 based on a pre-determined humidity (or a pre-determined humidity range) of at least one electrode of the defibrillators 106 within the storage sections 250. In other examples, the environmental controller 240 controls the humidity of the storage sections 250 based on a pre-determined humidity (or a pre-determined humidity range) of the defibrillator's manufacturer's recommendation within the storage sections 250. In this example, the deployment station 200 queries the database of the server 120 and/or the Internet to determine the manufacturer's recommended humidity range for the make/model/type of the defibrillator 106 (e.g., as determined via the identification information) and sets the pre-determined target humidity range to be the recommended humidity range, e.g., between 0-60% humidity. In other scenarios, the deployment station 200 sets the pre-determined target humidity range to a different humidity range.

In some embodiments, the access controls 205 include the keypad 210, the card reader 214, the locking mechanism 208, the contact sensor 209, and the RFID reader 216. Aspects of the access controls 205 are described above with reference to FIG. 2A and are not repeated here.

In some embodiments, the communication hardware 207 includes the RFID reader 280, the microphone 231, the camera 281, the light 283, the transceiver 241, and a GPS receiver 243. Some aspects of the access controls 205 are described above with reference to FIGS. 2A and 2B and those aspects are not repeated here.

In some examples, the transceiver 241 communicates with corresponding transmitters, receivers, and/or transmitters of the emergency medical treatment and guidance apparatuses 104 and/or the defibrillators 106 of the deployment station 200 directly (e.g., as shown using communication link 233). In this scenario, the deployment station 200 can receive the identification information and/or readiness information directly from the emergency medical treatment and guidance apparatuses 104 and/or the defibrillators 106 (e.g., using RF communications such as Wi-Fi and/or Bluetooth), when the respective device includes electronics to transmit information via these technologies. For example, an emergency medical treatment and guidance apparatus 104 equipped with a built-in tablet device (e.g., emergency medical treatment and guidance apparatuses 500 and 600 as shown in FIGS. 5A, and 6, respectively) includes a transceiver for communicating using these wireless technologies with the deployment station 200.

In some examples, the RFID reader 280, the microphone 231, the transceiver 241, and/or the camera 281 is an “at least one sensor” (or generally a “sensor”) for obtaining identification information and/or readiness information of the emergency medical treatment and guidance apparatuses 104 and/or the defibrillators 106 within the deployment station 200. For example, the sensor can be the RFID reader 280 for reading RFID tags 252 of the emergency medical treatment and guidance apparatuses 104, defibrillators 106, and/or fire extinguisher 221. In other examples, the sensor can be the camera 281 for imaging the housings of the emergency medical treatment and guidance apparatuses 104, defibrillators 106, and/or fire extinguisher 221. In other examples, the sensor can be the microphone 231 for listening to an audible signal from the emergency medical treatment and guidance apparatuses 104, defibrillators 106, and/or fire extinguisher 221. In other examples, the sensor can be the transceiver 241 for receiving direct wireless communication from the emergency medical treatment and guidance apparatuses 104, defibrillators 106, and/or fire extinguisher 221.

FIG. 3 is a flowchart of a computer-implemented method 300 performed by a deployment station in accordance with some embodiments. In some embodiments, the method 300 is performed by the controller 203 (e.g., the at least one processor) of the deployment station 200, but the method can be performed by any of the deployment stations described herein. The method is described with reference to deployment station 200 for context.

The deployment station 200 obtains 302 readiness information of at least one emergency medical treatment and guidance apparatus 104 and at least one defibrillator 106 (e.g., via at least one sensor or via the database). In some examples, at least one sensor (e.g., one or more of the RFID reader 280, the camera 281, the microphone 231, and the transceiver 241, amongst others) obtains the readiness information from the at least one emergency medical treatment and guidance apparatus 104 and/or the at least one defibrillator 106.

In this context, the deployment station 200 uses “readiness information” to, at least in part, determine a readiness of the at least one emergency medical treatment and guidance apparatus 104 and/or the at least one defibrillator 106 within the deployment station 200. For example, the camera 281 can acquire an image of the readiness indicator 247 of the at least one emergency medical treatment and guidance apparatus 104 and/or the at least one defibrillator 106, and the camera 281 (or the controller 203) can perform image processing to determine a readiness based on the processed image of the readiness indicator 247.

In some examples, the sensor continuously acquires readiness information (e.g., an active RFID communication can always be on, the microphone 231 can always be listening, etc.). In other examples, the sensor periodically acquires readiness information (e.g., the camera 281 can automatically acquire images every hour, every day, etc.). In other words, the method 300 can be repeated continuously or periodically (e.g., hourly, daily, and weekly, amongst others).

In some embodiments, the deployment station 200 obtains identification information of the at least one emergency medical treatment and guidance apparatus 104 and/or the at least one defibrillator 106 (e.g., via at least one sensor or via the database). In some examples, the identification information is obtained using the same sensor that obtained the readiness information. In some examples, the identification information is obtained from the at least one emergency medical treatment and guidance apparatus 104 and/or the at least one defibrillator 106.

In this context, the deployment station 200 uses “identification information” to, at least in part, uniquely identify the at least one emergency medical treatment and guidance apparatus 104 and/or the at least one defibrillator 106. For example, the camera 281 can acquire an image of the housing of the respective device, and the camera 281 (or the controller 203) can perform imaging processing to determine at least part of an identity of the device (e.g., determine the device type based on comparing a features of the image with a database of images of known devices). In this scenario, the deployment station 200 can query the database of the server 120 and filter all devices within the readiness management system 100 with this particular device type to assist in determining the particular device. As another example, the camera 281 can acquire an image of the machine-readable codes 222 of the respective device and perform image processing to determine, using the camera 281 (or the controller 203), information encoded within the machine-readable code 222 and/or access a particular address of the database with identification information using a hyperlink embedded within the machine-readable code 222.

In this way, the deployment station 200 identifies the at least one emergency medical treatment and guidance apparatus 104 and the at least one defibrillator 106 based on the identification information obtained from the sensor (e.g., by performing image processing on the images to determine the identity of the respective device, by decoding audible messages to determine the identity of the respective device, amongst other methods). In some examples, the identification information is sufficient to uniquely identify the at least one emergency medical treatment and guidance apparatus 104 and/or the at least one defibrillator 106 (e.g., when a serial number is received via an RFID tag 252 of a respective device, when the serial number is received from the database directly, amongst other methods).

Once at least some readiness information is known, the deployment station 200 (e.g., the controller 203) determines 304 readiness of the at least one emergency medical treatment and guidance apparatus 104 and/or the at least one defibrillator 106 based on the obtained readiness information. For example, the deployment station 200 can determine readiness (e.g., determine whether the respective device is fully ready, needs inspection and is fit for use, or needs inspection yet is unfit for use; or determine whether the respective device is ready or simply needs inspection) based on an acquired image of the readiness indicator. In this example, the readiness information is the “image” and the determined readiness is the result of “ready” or “needs inspection.”

For example, in some embodiments, determining readiness includes determining whether a battery of the at least one emergency medical treatment and guidance apparatus 104 and/or the at least one defibrillator 106 has a battery charge above pre-determined threshold (e.g., 70%, 80%, amongst others). In this example, the readiness information is the “battery charge” and the battery charge information can be received by the deployment station 200 (e.g., the controller 203) either by receiving this information directly from the at least one emergency medical treatment and guidance apparatus 104 and/or the at least one defibrillator 106 or by determining the battery charge information using the battery sensor 274. In some examples, the battery is (or includes) a battery of a touch screen user interface of the at least one emergency medical treatment and guidance apparatus 104 and/or the at least one defibrillator 106. In some examples, the readiness is based on the battery status (e.g., remaining charge, health, usage, among others) of one or more batteries of the at least one emergency medical treatment and guidance apparatus 104 and/or the at least one defibrillator 106.

In some embodiments, the readiness is based on the measured temperature and the measured humidity within at least one of the storage sections 250 of the housing 204. For example, the readiness can be based on whether the measured temperature and/or the measured humidity of the storage sections 250 are outside of pre-determined target ranges (e.g., below 65 degrees Fahrenheit or above 75 degrees Fahrenheit). In this example, the deployment station 200 (e.g., the controller 203) would determine the readiness to be “needs inspection” when the measured temperature is 60 degrees Fahrenheit despite being otherwise ready.

In some examples, the pre-determined target ranges are different for the at least one emergency medical treatment and guidance apparatus 104 and the at least one defibrillator 106. In some examples, the readiness is based on whether the measured temperature and/or the measured humidity are outside of pre-determined target ranges for at least one pharmaceutical within the at least one emergency medical treatment and guidance apparatus 104. In some examples, the readiness is based on whether the measured temperature and/or the measured humidity are outside of pre-determined ranges for at least one electrode of the at least one defibrillator 106.

In some embodiments, determining readiness of the at least one emergency medical treatment and guidance apparatus 104 and/or the at least one defibrillator 106 includes determining how an inventory of medical supplies within the at least one emergency medical treatment and guidance apparatus 104 compares with an expected inventory representing a fully stocked emergency medical treatment and guidance apparatus 104. For example, the deployment station 200 may obtain information that 10 RFID tags are present within a particular emergency medical treatment and guidance apparatus 104 (e.g., by detecting the RFID tags directly using the RFID reader 280 of the deployment station 200 or by receiving RFID information directly from the particular emergency medical treatment and guidance apparatus 104). In some examples, the 10 RFID tags represents 10 medical supplies. The deployment station 200 can query the database of the server 120 to determine how many items are expected in a fully stocked emergency medical treatment and guidance apparatus 104 and if the expected number is not 10, the deployment station 200 would determine the readiness of the particular emergency medical treatment and guidance apparatus 104 to be “needs inspection.” Yet, as discussed herein, when the emergency medical treatment and guidance apparatus 104 is determined to need inspection, the emergency medical treatment and guidance apparatus's readiness indicator 247 may optionally further indicate whether the apparatus 104 is still fit for use or is unfit (or unsafe) for use.

In this example, the deployment station 200 determines how the inventory of the medical supplies within the particular emergency medical treatment and guidance apparatus 104 compares with the expected inventory representing a fully stocked emergency medical treatment and guidance apparatus 104. In some examples, this process includes (i) determining that the inventory of the medical supplies matches the expected inventory, and/or (ii) determining that at least a portion of the inventory of the medical supplies is missing.

In some embodiments, determining readiness of the at least one emergency medical treatment and guidance apparatus 104 and/or the at least one defibrillator 106 includes receiving an indication from the at least one defibrillator 106 regarding whether the at least one defibrillator 106 has passed a self-diagnostic test. For example, a deployment station 200 receiving an indication that a defibrillator 106 has failed a self-diagnostic test (or in some examples, has failed at least one aspect of a self-diagnostic test) would determine the readiness of the particular defibrillator 106 to be “needs inspection.” In cases where a defibrillator 106 has failed a self-diagnostic test (e.g., is unable to safely deliver a defibrillation shock), then the defibrillator's readiness indicator 247 may optionally indicate that the defibrillator 106 is further unfit (or unsafe) for use.

In some embodiments, determining readiness of the at least one emergency medical treatment and guidance apparatus 104 and/or the at least one defibrillator 106 includes querying the database of the server 120 to determine if a status report indicates a use of the at least one emergency medical treatment and guidance apparatus 104 and/or the at least one defibrillator 106. For example, the database receives a status report from the deployment station 200 when an emergency medical treatment and guidance apparatus 104 and/or a defibrillator 106 is used during a medical emergency. The status report includes information of when the particular emergency medical treatment and guidance apparatus 104 and/or the particular defibrillator 106 was used and which medical supplies from the particular emergency medical treatment and guidance apparatus 104 were used. In some examples, the status report includes information exchanged in the interactive query with the user 230. For example, a deployment station 200 receiving an indication that a defibrillator 106 has been used would determine the readiness of the particular defibrillator 106 to be “needs inspection,” unless the particular defibrillator 106 has been inspected since the particular defibrillator 106 was used (e.g., inspection and/or service information is included in the database and the deployment station 200 can retrieve this inspection information from the database).

In some embodiments, the readiness is based on a decoded audible message received by the microphone 231 of the deployment station 200. For example, a deployment station 200 receiving an audible message that indicates that a particular defibrillator 106 “needs inspection” causes the deployment station 200 to determine the readiness of the particular defibrillator 106 to be “needs inspection.”

In some embodiments, the readiness is based on an image of individual readiness indicators 247 of the at least one emergency medical treatment and guidance apparatus 104 and/or the at least one defibrillator 106. For example, a deployment station 200 can acquire an image of the individual readiness indicators 247 using the camera 281 of the deployment station 200. In examples where the individual readiness indicator 247 of a particular emergency medical treatment and guidance apparatus 104 shows a green checkmark, the deployment station 200 (e.g., the camera 281 and/or the controller 203) would perform imaging processing on the image to determine this is a green checkmark (e.g., via image processing) and subsequently determine the readiness of the particular defibrillator 106 to be “ready.”

In some embodiments, the readiness of the at least one emergency medical treatment and guidance apparatus 104 and/or the at least one defibrillator 106 is based on the imaged machine-readable code 245 of the at least one emergency medical treatment and guidance apparatus 104 and/or the at least one defibrillator 106. For example, a deployment station 200 can acquire an image of the machine-readable code 245 using the camera 281 of the deployment station 200. In examples where the machine-readable code 245 of a particular emergency medical treatment and guidance apparatus 104 encodes readiness information indicating that the device is “ready,” (or otherwise provides a hyperlink to an address in the database where readiness information can be retrieved), the deployment station 200 determine the readiness of the particular emergency medical treatment and guidance apparatus 104 to be “ready.”

In some embodiments, the deployment station 200 updates 306 the readiness indicator 220 based on the determined readiness of the at least one emergency medical treatment and guidance apparatus 104 and/or the at least one defibrillator 106. In this example, the readiness indicator 220 is changed from “ready” to “needs inspection” (or variants thereof, such as providing information as to whether devices of the deployment station 200 are present, present and usable and/or absent) when at least one emergency medical treatment and guidance apparatus 104 and/or at least one defibrillator 106 has a readiness of “needs inspection.”

In some embodiments, the readiness indicator 220 and/or the machine-readable code 222 are dynamically updated (e.g., periodically updated) based on the determined readiness of the at least one emergency medical treatment and guidance apparatus 104 and/or the at least one defibrillator 106. For example, the readiness indicator 220 and/or the machine-readable code 222 can be periodically updated (e.g., minutely, hourly, daily, and/or the like) to indicate changes to the readiness of the devices within the deployment station 200. For example, the controller 203 determines the readiness as described above for each device within the deployment station 200 and can determine an overall readiness (e.g., if at least one device needs inspection, the overall readiness is “needs inspection”). The frequency of each readiness determination may be different for each device depending on the frequency that the individual readiness indicator 247 and/or the machine-readable codes 245 is updated for each respective device. For example, a first emergency medical treatment and guidance apparatus 104 may update individual readiness indicator 247 at a low frequency (e.g., daily) to conserve battery power, while a different first emergency medical treatment and guidance apparatus 104 may update individual readiness indicator 247 at a higher frequency (e.g., hourly) because it may include a larger battery or may include medical supplies that require closer inspection (e.g., pharmaceuticals that should not be exposed to temperatures outside of the pre-determined temperature ranges). In some embodiments, the deployment station 200 is configured to be in electrical communication with the housed at least one emergency medical treatment and guidance apparatus 104 and at least one defibrillator 106, and thus may update the readiness information of the respective at least one emergency medical treatment and guidance apparatus 104 and/or at least one defibrillator 106 in real-time. Alternatively, and as discussed further herein, the deployment station 200 is equipped with one or more sensors that are able to detect readiness information from the corresponding at least one emergency medical treatment and guidance apparatus 104 and/or at least one defibrillator 106.

In some embodiments, the deployment station 200 transmits 308 a readiness signal representing the readiness of the at least one emergency medical treatment and guidance apparatus 104 and/or the at least one defibrillator 106 to the server 120 for maintaining the database of the readiness of the at least one emergency medical treatment and guidance apparatus 104 and/or the at least one defibrillator 106 (e.g., the devices within the deployment station 200).

FIGS. 4A and 4B show a defibrillator 400 with a readiness indicator 402 in accordance with some embodiments. FIG. 4A is a front plan view and FIG. 4B is a schematic view of the defibrillator 400 inside a deployment station 450 as part of a readiness management system, such as the readiness management system 100 described with reference to FIG. 1. In some embodiments, the defibrillator 400 is the same as, or similar to, the defibrillator 106 described with reference to FIG. 1.

In the example shown in FIGS. 4A and 4B, the defibrillator 400 includes an AED that includes electronic circuitry to administer electrotherapy to a patient. For example, the defibrillator 400 includes a pair of defibrillation electrodes 404 and at least one high-voltage capacitor (not shown) for delivering an electrical shock to a patient. The defibrillator 400 includes an RFID tag 430 readable by an RFID reader 470 of the deployment station 450. The defibrillator 400 includes a machine-readable code 440 readable by the RFID reader 470 of the deployment station 450. The defibrillator 400 includes a user interface and display 416 for presenting medical instructions to a user. The defibrillator 400 includes the readiness indicator 402 on a housing of the defibrillator 400.

As shown in FIG. 4B, the defibrillator 400 also includes electrotherapy controls 412, self-diagnostic controls 414, a controller 420, and a transceiver 422. The transceiver 422 is able to transfer information to and/or from a transceiver 460 of the deployment station 450, to and/or from a server such as the server 120, and to and/or from a mobile device 480 associated with a user, as illustrated via various communication links 490.

In the embodiment shown, the readiness indicator 402 is a two state indicator showing a “check” or an “x.” Generally, the “check” is associated with a “ready” state and the “x” is associated with a “needs inspection” state or state that indicates the device is unfit or unsafe for use. In some embodiments, the defibrillator 400 (e.g., the controller 420) determines whether the defibrillator 400 is ready or needs inspection based on results of a self-diagnostic test. In this scenario, the self-diagnostic controls 414 perform a self-diagnostic test to determine a status of the defibrillator 400, and the defibrillator 400 (e.g., the controller 420) controls the readiness indicator 402 based on the determined status from this self-diagnostic test.

In some examples, the self-diagnostic test verifies that a defibrillator charge and discharge capability is functioning properly by determining when one or more of the following conditions are satisfied. In some examples, the defibrillator 400 (e.g., the self-diagnostic controls 414) verifies that the electrodes 404 are properly connected to the defibrillator 400 (e.g., via a resistance continuity check). In some examples, the defibrillator 400 (e.g., the self-diagnostic controls 414) verifies that the ECG signal acquisition and processing electronics of the electrotherapy controls 412 are functional. In some examples, the defibrillator 400 (e.g., the self-diagnostic controls 414) verifies that the electrotherapy controls 412 can charge and discharge at up to 200 joules. In some examples, the defibrillator 400 (e.g., the self-diagnostic controls 414) verifies proper function of the Fully Automatic AED Plus microprocessor electronics of the electrotherapy controls 412 and the integrity of the software running on the microprocessor. In some examples, the defibrillator 400 (e.g., the self-diagnostic controls 414) verifies that CPR monitoring and compression depth detection are functional. In some examples, the defibrillator 400 (e.g., the self-diagnostic controls 414) verifies that voice prompts are functional by transmitting an audible request using a speaker of the defibrillator 400 (e.g., the self-diagnostic controls 414) and listening for the audible request using a microphone of the defibrillator 400 (e.g., the self-diagnostic controls 414). One or more of these functions may be provided in a self-diagnostic test to indicate whether the defibrillator 400 is safe for use or is unsafe for use.

FIGS. 5A and 5B show a portable emergency medical treatment and guidance apparatus 500 with two readiness indicators 502 and 503 in accordance with some embodiments. FIG. 5A is a perspective view and FIG. 5B is a schematic view of the emergency medical treatment and guidance apparatus 500 within a deployment station 550 as part of a readiness management system, such as the readiness management system 100 described with reference to FIG. 1. In some embodiments, the emergency medical treatment and guidance apparatus 500 is the same as, or similar to, the emergency medical treatment and guidance apparatus 104 described with reference to FIG. 1.

Examples of such portable emergency medical treatment and guidance apparatuses 500 are the Mobilize Comprehensive Rescue System by ZOLL Medical Corporation of Chelmsford, Massachusetts.

Each emergency medical treatment and guidance apparatus 500 includes a case 506 (i.e., a housing) with a plurality of medical supplies 508 (or medical items) for administering medical treatment (e.g., but not limited to, 4× SOF-T Wide tourniquet, 2× QuikClot bleeding control dressing, 2×6″ flat emergency trauma dressing, 2× Hyfin chest seal, 2× Water-Jel universal burn dressing, 2× triangular bandage, 2×4.5″ sterile conforming stretch gauze, 4×5″×9″ sterile combine ABD pads, 2×10″×30″ sterile multi-trauma dressing, a 36″ SAM emergency splint, 2×4″ elastic wrap bandage, a 4″×5″ cold compress, an adhesive tape 2.5 yd, a CPR face shield with bite block, a 81 mg chewable aspirin (bottle), a 9 mg dissolvable allergy tablets (box), a 15 mg Insta-Glucose, 2× emergency space Mylar blanket, a portable charger and charging cord, a USB charging cube, an inspection card, 9× proof seals, 2× bag with biohazard markings, a pair of trauma shears, 10× nitrile gloves, a user manual and inventory card, 2 eye pads, 16× adhesive bandages (assorted sizes), 10× burn cream packets, 10× triple antibiotic ointment packets, 10× antiseptic wipes, a pair of tweezers, 10× hand sanitizer packets, an eye wash, and a first aid guide). The medical supplies 508 are located within an inside compartment 510 of the emergency medical treatment and guidance apparatus 500.

The emergency medical treatment and guidance apparatus 500 includes a built-in tablet device 504 housed within the case 506 of the emergency medical treatment and guidance apparatus 500. The tablet device 504 includes a touch-screen for providing an interactive query to a user. The tablet device 504 is configured to run an application (e.g., the ZOLL Mobilize Rescue App) to provide real-time instructions (i.e., during the medical treatment) to help caregivers assess, manage, and treat victims/patients in emergency situations. Further aspects of such applications are described with reference to FIGS. 11A and 11B below.

The emergency medical treatment and guidance apparatus 500 includes two readiness indicators 502 and 503. In some examples, one readiness indicator 502 is used to communicate readiness of the tablet device 504 and/or the software application configured to run on the tablet device 504, while the other readiness indicator 503 is used to communicate readiness of the medical supplies 508 within the emergency medical treatment and guidance apparatus 500. In this way, a first readiness indicator 502 indicates the readiness of the tablet device 504 and/or software application and a second readiness indicator 503 indicates the readiness of the medical supplies 508. However, while two readiness indicators 502 and 503 are used with the emergency medical treatment and guidance apparatus 500, other emergency medical treatment and guidance apparatuses can use one readiness indicator (e.g., readiness indicator 502) to indicate the readiness of both the tablet device 504 and/or software application and the medical supplies 508 within the emergency medical treatment and guidance apparatus 500. In fact, while not explicitly shown for the other emergency medical treatment and guidance apparatuses described with reference to FIGS. 6-7B, some examples of those emergency medical treatment and guidance apparatuses can include two readiness indicators.

As shown in FIG. 5B, the emergency medical treatment and guidance apparatus 500 includes one or more RFID readers 520 similar to, or the same as, the RFID reader 280 of the deployment station 200 described with reference to FIGS. 2A-2C. Similarly, each of the medical supplies 508 may include an RFID tag 522 readable by the one or more RFID readers 520 of the emergency medical treatment and guidance apparatus 500. In the example shown, the emergency medical treatment and guidance apparatus 500 includes four medical supplies 508 within the compartment 510: an A1-Touniquet, a C1-Chest Seal, a B1-QuickClot, and Gloves.

In some examples, each of these medical supplies 508 has a respective RFID tag 522. The one or more RFID readers 520 read the RFID tags 522 and determines the identity of the medical supplies 508 from the read RFID information. In this way, the one or more RFID readers 520 of the emergency medical treatment and guidance apparatus 500 read the RFID tags 522 of the medical supplies 508 analogously to how the RFID reader 280 of deployment station 200 reads the RFID tags 252 of the emergency medical treatment and guidance apparatuses 106 as described with reference to FIGS. 2A-2C. In some examples, the one or more RFID readers can read the RFID tags 522 of the medical supplies 508 directly. This functionality is described above with reference to FIG. 3. The emergency medical treatment and guidance apparatus 500 includes an RFID tag 530 that is readable by the one or more RFID readers 520 of the deployment station 550. The one or more RFID readers 520 read the RFID tag 530, and the emergency medical treatment and guidance apparatus 500 (e.g., controller 524 thereof) determine the identity of the emergency medical treatment and guidance apparatus 500 from the read RFID information.

The readiness indicator 502 is exposed on the outer surface of the housing 506 of the emergency medical treatment and guidance apparatus 500 and is optionally a two-state indicator that toggles between illuminating an LED on the left of the readiness indicator 502 vs. illuminating an LED on the right of the readiness indicator 502. In certain embodiments, the readiness indicator 502 may provide more information than simply two states, for example, where the information may include whether the emergency medical treatment and guidance apparatus 500 is fit for use and/or inventory information of the contents stored therein. For example, the readiness indicator 502 can be a micropower (<0.1 mW) LCD or electrophoretic (e.g., e-Ink) display in electrical communication with the controller 524 of the emergency medical treatment and guidance apparatus 500. The display may be configured to display graphically-encoded digital information, for instance as QR-codes or bar-codes, so that multiple bytes of readiness and test result information can be communicated with the deployment station 550, and thereby transmitted to the personnel managing the system by the personnel using, e.g., a mobile device 580, to scan the graphically-encoded digital information and display the readiness and test result information and/or transmit the readiness and test result information to the server 120. In some examples, multiple frames of the graphically-encoded digital information may be displayed in succession to increase informational content. The emergency medical treatment and guidance apparatus 500 includes a transceiver 522 for transmitting information. For example, the transceiver 522 is able to transfer information to and/or from a transceiver 560 of the deployment station 550, to and/or from the server 120, and to and/or from a mobile device 580 associated with a user, as illustrated via various communication links 590.

FIG. 6 shows a portable emergency medical treatment and guidance apparatus 600 with a user interface 604 (e.g., a removable tablet device) and a readiness indicator 602 in accordance with some embodiments. The emergency medical treatment and guidance apparatus 600 is substantially similar to the emergency medical treatment and guidance apparatus 500 described with reference to FIGS. 5A and 5B, except uses the user interface 604 instead of a built-in tablet. The emergency medical treatment and guidance apparatus 600 also includes a soft housing 608 (e.g., a soft backpack style housing as shown) as opposed to a hard (or substantially rigid) housing 506 of the emergency medical treatment and guidance apparatus 500. The emergency medical treatment and guidance apparatus 600 also includes an RFID tag 606 readable by an RFID reader of a deployment station, and includes a readiness indicator 602.

FIGS. 7A and 7B show a portable emergency medical treatment and guidance apparatus 700 with a readiness indicator 702 in accordance with some embodiments. In some examples, the emergency medical treatment and guidance apparatus 700 is part of a multi-pack system that includes four emergency medical treatment and guidance apparatuses 700 for treating medical emergencies (the four-pack system is not shown). Examples of such multi-pack systems are the Mobilize Public Access Rescue System of ZOLL Medical Corporation of Chelmsford, Massachusetts.

In certain embodiments, each emergency medical treatment and guidance apparatus 700 includes a case 708 (i.e., a housing) with a plurality of medical supplies 712 (or medical items) for administering medical treatment (e.g., but not limited to, a SOF-T Wide tourniquet, a 4″ emergency trauma dressing, a QuikClot bleeding control dressing, a Hyfin chest seal, a CPR face shield with bite block, an emergency space Mylar blanket, a trauma shears, and 10× nitrile gloves). Examples of such emergency medical treatment and guidance apparatuses 700 are the ZOLL Mobilize Public Access Utility Kit and the ZOLL Mobilize Public Access Compact Rescue System. The housing 708 is a soft fabric material and includes a zipper to seal the contents of the housing 708.

The emergency medical treatment and guidance apparatus 700 includes an RFID tag 704 readable by an RFID reader of a deployment station, and includes a readiness indicator 702. The readiness indicator 702 is exposed on an outer surface of the housing 708 of the emergency medical treatment and guidance apparatus 700 and is optionally a two-state indicator that toggles between illuminating an LED on the left of the readiness indicator 702 vs. illuminating an LED on the right of the readiness indicator 702. In certain embodiments, the readiness indicator 702 may provide more information than simply two states, for example, where the information may include whether the apparatus is fit for use and/or inventory information of the contents stored therein. In some examples, a ready for use indication of the emergency medical treatment and guidance apparatus 700 depends on whether the emergency medical treatment and guidance apparatus 700 includes no expired medical supplies.

In some examples, a mobile device of a user is configured to execute/run an application for providing instructions to a caregiver for administering medical treatment using the emergency medical treatment and guidance apparatus 700. For example, a mobile device (e.g., the user's smartphone) can run an application (e.g., ZOLL Mobilize Rescue App) to provide real-time instructions (i.e., during the medical treatment) to help caregivers assess, manage, and treat victims/patients in emergency situations. Such an application is compatible with any of the emergency medical treatment and guidance apparatus described herein.

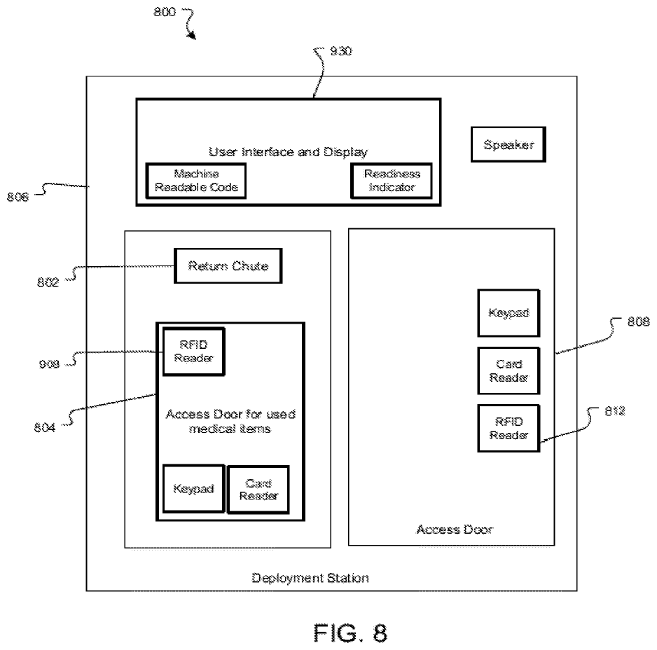

FIG. 8 shows a schematic of a deployment station 800 in accordance with some embodiments with a return chute 802 and an access door 804 for returning used emergency medical treatment and guidance apparatuses and used defibrillators. In some embodiments, the deployment station 800 is substantially similar to, or the same as, the deployment station 200 except that the deployment station 800 includes features for returning used emergency medical treatment and guidance apparatuses and used defibrillators.

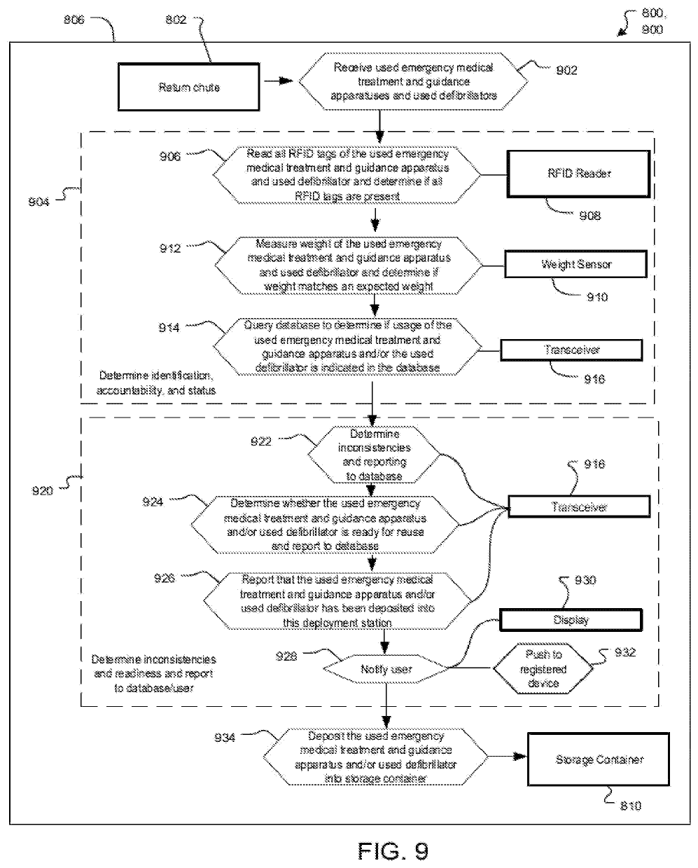

In some examples, a user returns used emergency medical treatment and guidance apparatuses and used defibrillators by depositing the used devices into the return chute 802. The return chute 802 is mechanically connected to a duct that transports the used emergency medical treatment and guidance apparatuses and used defibrillators into a storage container 810 (shown in FIG. 9) within the deployment station's housing 806 (e.g., storage container 810 may be located behind the access door 804 for returning used emergency medical treatment and guidance apparatuses and used defibrillators). For example, when an inspector opens the access door 804 for returning used emergency medical treatment and guidance apparatuses and used defibrillators (e.g., via a passcode, card, or RFID tag as described for the access door 202 in FIG. 2A), the inspector can reach into the storage container 810 and can physically take the used emergency medical treatment and guidance apparatuses and used defibrillators from the storage container 810. In some examples, the inspector will prepare the used emergency medical treatment and guidance apparatuses and used defibrillators to be used again by sanitizing and/or servicing the used defibrillators, replacing missing medical supplies from the emergency medical treatment and guidance apparatuses, among others.

In some embodiments, the deployment station's access door 808 is substantially the same as the access door 202 of deployment station 200. In some examples, at least one emergency medical treatment and guidance apparatus and at least one defibrillator is stored within a storage section within the housing 806 behind the access door 808. In this way, the deployment station 800 includes all the functionality of deployment station 200 but also includes features to store used emergency medical treatment and guidance apparatuses and used defibrillators.

FIG. 9 shows a flowchart of a process 900 performed by the deployment station 800 in accordance with some embodiments for returning used emergency medical treatment and guidance apparatuses and used defibrillators. The process 900 includes receiving 902 the used emergency medical treatment and guidance apparatuses and used defibrillators (e.g., by a user depositing the used receive used emergency medical treatment and guidance apparatuses and used defibrillators in the return chute 802). The process 900 can include several steps for determining 904 an identification of each medical item within the used emergency medical treatment and guidance apparatuses (e.g., tourniquet, gloves, among others) and/or each medical item of the defibrillator (e.g., electrodes, among others) to determine if everything is accounted for. In some examples, the deployment station 800 determines 904 the identification by reading 906 all RFID tags of the used emergency medical treatment and guidance apparatus and/or used defibrillator.

For example, in the case of a used and returned emergency medical treatment and guidance apparatus, the deployment station 800 reads an RFID tag on the outside housing of the used and returned emergency medical treatment and guidance apparatus and all the RFID tags for the medical supplies within the used emergency medical treatment and guidance apparatus (e.g., the tourniquet, the gloves, among others) using an RFID reader 908 of the deployment station 800, which in this illustrated embodiment is located on the access door 804. The deployment station 800 queries a database of a server (e.g., the database of the server 120), e.g., using a transceiver 916 of the deployment station 800, to compare the read RFID tags for the particular used and returned emergency medical treatment and guidance apparatus RFID information with the database information to determine if all medical supplies are accounted for. The database information identifies all of the medical supplies that should be present in the emergency medical treatment and guidance apparatus or defibrillator as fully stocked. The comparison therefore allows any medical items missing from the used and returned emergency medical treatment and guidance apparatus (or the used and returned defibrillator) to be identified since RFID tags for medical supplies will be read for only those medical supplies that are present in the used and returned emergency medical treatment and guidance apparatus (or the used and returned defibrillator). For example, the deployment station 800 may determine that a roll of gauze is missing from the used and returned emergency medical treatment and guidance apparatus based on this RFID information or that electrodes are missing from the used and returned defibrillator. In some examples, readiness of the used and returned emergency medical treatment and guidance apparatus is based on whether all RFID tags are determined to be present, which is indicative of all medical items that should be present in the emergency medical treatment and guidance apparatus or defibrillator as fully stocked being present in the used and returned emergency medical treatment and guidance apparatus since RFID tags have been read by the deployment station 800 to account for all of the medical items.

In some examples, the deployment station 800 receives detailed identification information about the used and returned emergency medical treatment and guidance apparatus and/or used and returned defibrillator based on the RFID information. For example, the deployment station 800 queries the database with the RFID information to retrieve detailed information such as the expected inventory, last used dates, last inspection dates, expiration dates of pharmaceuticals, expiration dates of batteries, expiration of electrodes, among others.

The deployment station 800 transmits information to the server (e.g., the server 120) that the used and returned emergency medical treatment and guidance apparatus and/or the used and returned defibrillator is located within the deployment station 800 (e.g., within the storage container 810). In this example, the server can update the location information of the particular used and returned emergency medical treatment and guidance apparatus and/or the used and returned defibrillator within the database so that other queries to the database will indicate that the particular used and returned emergency medical treatment and guidance apparatus and/or the used and returned defibrillator is stored within deployment station 800.

In some examples, the deployment station 800 at least in part determines 904 the identification by weight. In this scenario, the deployment station 800 measures 912 a weight of the used and returned emergency medical treatment and guidance apparatus and/or the used and returned defibrillator using a weight sensor 910 (e.g., a scale). In some examples, the weight sensor 910 is (or is part of) the at least one sensor.

In some examples, the weight sensor 910 is built-into a bottom of the storage container 810 such that the measured weight is incrementally increased as used emergency medical treatment and guidance apparatuses and/or used defibrillators are returned to the deployment station 800. In this way, the difference in weight is used (e.g., as measured from before vs. after the used emergency medical treatment and guidance apparatus and/or the used defibrillator is returned) as the weight of the used and returned emergency medical treatment and guidance apparatus and/or the used and returned defibrillator.

In other embodiments, the return chute 802 may transfer the used and returned emergency medical treatment and guidance apparatus and/or the used and returned defibrillator to an intermediate platform that includes the weight sensor 910 for measuring the weight of the used and returned emergency medical treatment and guidance apparatus and/or the used and returned defibrillator before the used and returned emergency medical treatment and guidance apparatus and/or the used and returned defibrillator reaches the storage container 810 or before the used and returned emergency medical treatment and guidance apparatus and/or the used and returned defibrillator falls as far as gravity will allow within the storage container 810. Once the weight is obtained, the intermediate platform mechanically actuates (e.g., tilts) to urge the used and returned emergency medical treatment and guidance apparatus and/or the used and returned defibrillator to continue traversing to the storage container 810 or to continue falling under the force of gravity within the storage container 810. The intermediate platform then returns to its original position to be ready for a next return.

The deployment station 800 can include a controller, as discussed herein with respect to various deployment stations, which can be operatively coupled to the weight sensor 810 and to an actuator operatively coupled to the intermediate platform. In response to the controller receiving a signal from the weight sensor 810 indicating that a weight has been measured, the controller can cause the actuator to be actuated, thereby moving the intermediate platform to urge the used and returned emergency medical treatment and guidance apparatus and/or the used and returned defibrillator to continue traversing to the storage container 810 or to continue falling under the force of gravity within the storage container 810. The actuator can include, for example, a magnetic actuator that the controller causes to move such that the intermediate platform is actuated (e.g., tilted) because the intermediate platform is no longer being held in position magnetically and then that the controller causes to move back to its original position to again hold the intermediate platform in position magnetically. For another example, the actuator can include a pivotable lever arm configured to be mechanically pivoted in a first direction to cause the intermediate platform to be actuated (e.g., tilted) and then mechanically pivoted in a second, opposite direction to return the intermediate platform to its original position.

In examples where the weight is determined 912, the weight is compared with expected weight information retrieved from the database of the server to determine if the measured weight is representative of an expected weight of the used and returned emergency medical treatment and guidance apparatus and/or the used and returned defibrillator. The expected weight information indicates weight of the emergency medical treatment and guidance apparatus and/or the defibrillator as fully stocked. The expected weight information can be specific to the returned emergency medical treatment and guidance apparatus and/or the returned defibrillator since identification information can be used to uniquely identify the returned emergency medical treatment and guidance apparatus and/or the returned defibrillator. For example, the RFID information may be used to determine the identity of the used and returned emergency medical treatment and guidance apparatus and/or the used and returned defibrillator and the measured weight used to determine if all the medical supplies are accounted for since, in general, a measured weight less than the expected weight indicates that not all of the medical supplies are present. This can be used in addition to or replacement of reading the RFID tags of each medical item individually.

In examples where the weight is determined 912, the deployment station 800 determines if the measured weight matches the expected weight. For example, the deployment station 800 determines whether the measured weight is within a pre-determined range of the expected weight based on comparing the measured weight with the expected weight of the used and returned emergency medical treatment and guidance apparatus and/or used and returned defibrillator. In some examples, readiness of the emergency medical treatment and guidance apparatus and/or the defibrillator is based on whether the weight is determined to be within the pre-determined range. For example, the used and returned emergency medical treatment and guidance apparatus and/or the used and returned defibrillator can be “ready” if the measured weight is within 5% of the fully stocked weight for that particular device.

The process 900 includes querying 914 the database to determine if usage of the used and returned emergency medical treatment and guidance apparatus and/or the used and returned defibrillator is indicated in the database. For example, the deployment station 800 queries the database via the transceiver 916 of the deployment station 800. In some examples, usage is indicated in the database by inferring use from a status report. In this scenario, after the user used the emergency medical treatment and guidance apparatus and/or the defibrillator for a medical emergency, information about the usage is compiled into a status report by the emergency medical treatment and guidance apparatus and/or the defibrillator and sent to the database, where the status information may be recorded by the deployment station 800 and then further sent to the database. In some examples, the determination of whether the used emergency medical treatment and guidance apparatus and/or used defibrillator was used in the medical emergency is performed by the server.

For example, once the deployment station 800 receives an indication that the used emergency medical treatment and guidance apparatus and/or the used defibrillator was used in a medical emergency, the deployment station 800 may query the database to determine when the medical emergency occurred to determine a likelihood that the used emergency medical treatment and guidance apparatus and/or the used defibrillator was used in that particular medical emergency. Furthermore, the deployment station 800 may also query the database to retrieve information about when the used emergency medical treatment and guidance apparatus and/or the used defibrillator was removed from the deployment station 800 to establish a range of dates when the medical emergency must have occurred in order for the used emergency medical treatment and guidance apparatus and/or the used defibrillator to have been used in the medical emergency. In some examples, readiness status is based on whether used and returned emergency medical treatment and guidance apparatus and/or used and returned defibrillator was used in a medical emergency. For example, the used and returned emergency medical treatment and guidance apparatus and/or the used and returned defibrillator can be “needs inspection” if the used and returned emergency medical treatment and guidance apparatus and/or used and returned defibrillator was used in a medical emergency because some items may be missing due to their use during the medical emergency.

The process 900 includes steps to determine 920 inconsistencies and readiness status of the used and returned emergency medical treatment and guidance apparatus and/or the used and returned defibrillator and report the inconsistencies and readiness status to the database of the server, a user interface and display 930 of the deployment station 800, and to a user/inspector (e.g., via a mobile device of the user/inspector). This process 900 includes determining 922 any inconsistencies and reporting the inconsistencies to the database (e.g., via the transceiver 916). For example, if one or more medical supplies are determined to be missing from the used and returned emergency medical treatment and guidance apparatus and/or the used and returned defibrillator (e.g., by comparing the expected inventory from the database with the actual inventory determined via the RFID reader 908 and, in some examples, the weight sensor 910), the deployment station 800 can report this inconsistency to the server for updating the database to indicate this inconsistency.

The process 900 includes determining 924 whether the used and returned emergency medical treatment and guidance apparatus and/or used and returned defibrillator is ready for reuse (e.g., the degree of readiness) and reporting the readiness status to the database. The readiness status is determined based on the inconsistencies, the weight, the RFID tag information, and generally any of the aspects of method 300 described with reference to FIG. 3 above.

The process 900 includes reporting 926 to the database that the used and returned emergency medical treatment and guidance apparatus and/or used and returned defibrillator has been deposited into the deployment station 800. For example, in response to receiving the report that the used and returned emergency medical treatment and guidance apparatus and/or used and returned defibrillator has been deposited into the deployment station 800, the server updates the database to reflect that the used and returned emergency medical treatment and guidance apparatus and/or used and returned defibrillator is at the location of the deployment station 800.

The process 900 includes notifying 928 the user/inspector of the inconsistencies, the readiness status, and that the used and returned emergency medical treatment and guidance apparatus and/or used and returned defibrillator is stored within the deployment station 800. In some examples, the deployment station 800 pushes a notification (e.g., via the transceiver 916) to registered devices (e.g., mobile devices) of the inspectors within the network (e.g., as shown in FIG. 1) with information about the inconsistencies, the readiness status of the used and returned emergency medical treatment and guidance apparatus and/or used and returned defibrillator, and that the used and returned emergency medical treatment and guidance apparatus and/or used and returned defibrillator is stored within the deployment station 800. In this way, the process 900 includes transmitting a signal representing the readiness to a mobile device such that the signal causes the mobile device to trigger a mobile alert indicating the readiness status of the used and returned emergency medical treatment and guidance apparatus and/or the used and returned defibrillator.

In some examples, the deployment station 800 displays the information about the inconsistencies, the readiness status of the used and returned emergency medical treatment and guidance apparatus and/or used and returned defibrillator, and that the used and returned emergency medical treatment and guidance apparatus and/or used and returned defibrillator is stored within the deployment station 800 on the user interface and display 930 of the deployment station 800 (also shown in FIG. 8).

The process 900 includes depositing 934 the used emergency medical treatment and guidance apparatus and/or used defibrillator into the storage container 810 when the used emergency medical treatment and guidance apparatus and/or used defibrillator is deposited by a user in the return chute 802. The used and returned emergency medical treatment and guidance apparatus and/or used and returned defibrillator resides in the storage container 810 until an inspector retrieves the used and returned emergency medical treatment and guidance apparatus and/or used and returned defibrillator from the storage container 810.

In examples where the used and returned emergency medical treatment and guidance apparatus and/or used and returned defibrillator is ready to be reused without requiring inspection (e.g., perhaps because the used and returned emergency medical treatment and guidance apparatus is still fully stocked, and/or because the used and returned defibrillator is fully functional), the deployment station 800 may allow a user to use the used and returned emergency medical treatment and guidance apparatus and/or used and returned defibrillator. For example, the deployment station 800 can present instructions on the user interface and display 930 instructing the user to take the used and returned emergency medical treatment and guidance apparatus and/or used and returned defibrillator from the storage container 810. The deployment station 800 can unlock the access door 804 to provide the user access to the storage container 810 in this scenario even when the user is not an inspector.

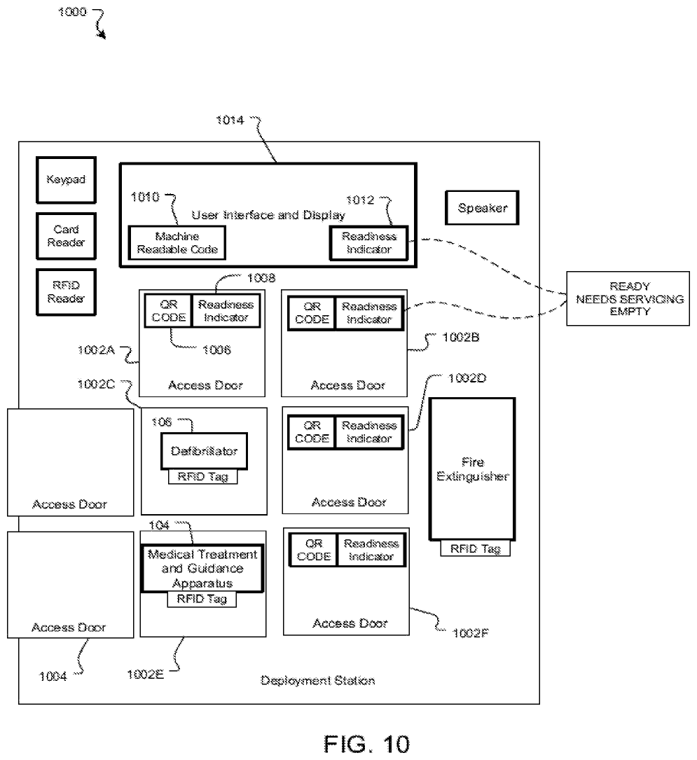

FIG. 10 shows a schematic of a deployment station 1000 in accordance with some embodiments with multiple storage sections 1002A, 1002B, 1002C, 1002D, 1002E, 1002F (generally “storage sections” 1002) for individually storing portable emergency medical treatment and guidance apparatuses and defibrillators, such as the portable emergency medical treatment and guidance apparatuses 104 and defibrillators 106. Each of the storage sections 1002A, 1002B, 1002C, 1002D, 1002E, 1002F can store therein at least one portable emergency medical treatment and guidance apparatus, at least one defibrillator, and/or a fire extinguisher (such as the fire extinguisher 221 of FIG. 2B). FIG. 10 shows one of the storage sections 1002C storing a defibrillator 106 therein and another one of the storage sections 1002E storing a portable emergency medical treatment and guidance apparatus 104 therein. In some embodiments, the deployment station 1000 is substantially similar to, or the same as, the deployment station 200 except that the deployment station 1000 includes individual storage sections 1002. In the example shown, the deployment station 1000 includes six individual storage sections 1002, but less than six (e.g., 1, 2, 3, 4, 5) or more than six (e.g., 7, 8, 10, among others) are possible with other embodiments.

In some embodiments, each storage section 1002 of the deployment station 1000 includes an access door 1004. FIG. 10 shows the access door 1004 closed for four of the storage sections 1002A, 1002B, 1002D, 1002F and open for two of the storage sections 1002C, 1002E. In some embodiments, each access door 1004 is substantially similar to, or the same as, the access door 202 of deployment station 200 except each access door 1004 includes a machine-readable code 1006 (in the form of a QR code in this illustrated embodiment) and a readiness indicator 1008 directly on the access door 1004. In some examples, the machine-readable code 1006 and the readiness indicator 1008 are displayed on a display (not explicitly shown).

In some examples, the display on the access door 1004 presents the machine-readable code 1006 encoding a hyperlink such that, when imaged by a mobile device, the hyperlink directs the mobile device to retrieve readiness information from a database hosted by a server (e.g., the database hosted by the server 120) and display information related to the readiness of the device within the particular storage section 1002 associated with that access door 1004. For example, when a mobile device images the machine-readable code 1006 presented on the access door 1004 of a fifth one of the storage sections 1002E (the machine-readable code 1006 is hidden from view because the access door 1004 is open), readiness information of the emergency medical treatment and guidance apparatus 104 is presented on the mobile device. Similarly, when a mobile device images the machine-readable code 1006 presented on the access door 1004 of a third one of the storage sections 1002C (also hidden from view because the access door 1004 is open), readiness information of the defibrillator 106 is presented on the mobile device. In this way, the machine-readable code 1006 is intended to provide a quick method for an inspector to see readiness information of the devices within each storage section 1002 with the access door 1004 in the closed position.

Similarly, the readiness indicator 1008 on each access door 1004 presents the readiness information for the devices within each storage section 1002. This enables an inspector to see which storage sections 1002 require attention by a glance of the deployment station 1000.

In some embodiments, the deployment station 1000 includes a user interface and display 1014 that is substantially the same as the user interface and display 218 of the deployment station 200. The user interface and display 1014 includes a machine-readable code 1010 and a readiness indicator 1012 representing all emergency medical treatment and guidance apparatus 104 and defibrillators 106 of the deployment station 1000. In this way, the machine-readable code 1010 and the readiness indicator 1012 is the same as the machine-readable code 222 and the readiness indicator 220 of deployment station 200.

FIG. 11 shows a screenshot of an application 1100 running on a user interface and display of a deployment station. For context, the description of FIG. 11 specifically refers to the deployment station 200 and the user interface and display 218 of the deployment station 200 as described with reference to FIGS. 2A-2C, but the application 1100 is operable with any of the deployment stations described herein.

In some embodiments, the application 1100 includes a first button 1102 (e.g., a touch screen button) to call emergency services (e.g., 911). For example, when a user presses the first button 1102 on the user interface and display 218, the deployment station 200 places a call to emergency services using the transceiver 241 of the deployment station 200.

In some embodiments, the application 1100 includes a second button 1104 (e.g., a touch screen button) to determine a medical need of a patient. For example, when a user presses the second button 1104 on the user interface and display 218, the user interface and display 218 provides an interactive query to determine the medical need of the patient and to recommend an emergency medical treatment and guidance apparatus 104 and/or a defibrillator 106 based on this determined medical need.

For example, determining the medical need can include presenting an image of a human body (not shown) on the user interface and display 218 and waiting for a response from a user/caregiver indicating an area on the human body where the patient is experiencing a problem, e.g., by the user/caregiver touching the area on the user interface and display 218, and/or providing an indication of the problem, e.g., by selecting an option in a drop-down menu, text input, etc. The instructions are provided visually via the user interface and display 218 and/or, in some instances, audibly via one or more speakers of the deployment station 200. The instructions include one or more questions (or queries) so the real-time instructions can be determined based on the caregivers response to the query. Examples of interactive queries are described in U.S. Application No. 63/002,962, entitled “Portable Medical Treatment Apparatus With Interactive Guidance And Cardiopulmonary Resuscitative Functionality” filed Mar. 31, 2020, and International Publication No. WO 2021/202292 entitled “Portable Medical Treatment Apparatus With Interactive Guidance And Cardiopulmonary Resuscitative Functionality” published Oct. 7, 2021, the disclosures of which are each hereby incorporated by reference in its entirety. For example, if the user provides input/response to the inquiry such that the application 1100 determines that the patient is experiencing heavy bleeding, the application 1100 can recommend that an emergency medical treatment and guidance apparatus 104 be taken from the deployment station 200 (e.g., the application 1100 controls the user interface and display 218, such as by transmitting a command to the controller 203 via the transceiver 241, to display a notice to “take an emergency medical treatment and guidance apparatus to the patient” and/or provide guidance such as graphics that depict what the particular item to be used looks like for the user to know which device(s) to take). In this way, the deployment station 200 recommends one of the at least one emergency medical treatment and guidance apparatuses 104 stored in the deployment station 200 based on the interactive query.

In other examples, if the user provides input/response to the inquiry such that the application 1100 determines that the patient is unconscious, the application 1100 can recommend that a defibrillator 106 be taken from the deployment station 200 (e.g., the application 1100 controls the user interface and display 218, such as by transmitting a command to the controller 203 via the transceiver 241, to display a notice to “take a defibrillator to the patient” and/or provide guidance such as graphics that depict what the particular item to be used looks like for the user to know which device(s) to take). In this way, the deployment station 200 recommends one of the at least one of the defibrillators 106 stored in the deployment station 200 based on the interactive query.

In other examples, if the user provides input/response to the inquiry such that the application 1100 determines that the patient is experiencing a medical need that requires both an emergency medical treatment and guidance apparatus and a defibrillator (e.g., the patient is experiencing heavy bleeding and is unconscious), the application 1100 can control the user interface and display 218, such as by transmitting a command to the controller 203 via the transceiver 241, to display a notice to “take both an emergency medical treatment and guidance apparatus and a defibrillator to the patient” and/or provide guidance such as graphics that depict what the particular item(s) to be used look like for the user to know which device(s) to take. In this way, the deployment station 200 recommends one of the at least one emergency medical treatment and guidance apparatuses 104 stored in the deployment station 200 and one of the at least one defibrillators 106 stored in the deployment station 200 based on the interactive query.

In some embodiments, after the medical need is determined via this process, the deployment station 200 can provide a recommendation (e.g., visually or audibly) regarding which devices to take from the deployment station 200 (e.g., whether the user should take one of the emergency medical treatment and guidance apparatuses 104 and/or one of the defibrillators 106) based on the medical need of the patient. Subsequently the deployment station 200 can monitor contents of the deployment station 200, using the RFID reader 280, to determine if the user actually takes the recommended device(s) (e.g., by sensing a change in the received RFID signals from the RFID tags 252 of the devices). For example, if the determined medical need is a bleeding patient and the user removes one of the defibrillators 106 from the deployment station 200 instead of one of the emergency medical treatment and guidance apparatuses 104, the deployment station 200 can display a notification on the user interface and display 218 that the user had taken the wrong item. In some examples, this notification is provided audibly as well.

In some embodiments, the recommendation can include taking different versions of the emergency medical treatment and guidance apparatus 104 (e.g., recommending that the user take an emergency medical treatment and guidance apparatus 500 instead of an emergency medical treatment and guidance apparatus 700). In some examples, this recommendation is based on the required medical item being included in one version of the emergency medical treatment and guidance apparatus 500 but not included in another version of the emergency medical treatment and guidance apparatus 700 (e.g., because the emergency medical treatment and guidance apparatus 700 is a smaller, more compact kit).

In some embodiments, the application 1100 includes a third button 1106 (e.g., a touch screen button) to retrieve an access code for the deployment station 200. For example, when a user presses the third button 1106 on the user interface and display 218, the user interface and display 218 provides a contact list of people to contact to obtain an access code. The controller 203 can have pre-stored therein (e.g., in the memory thereof) the contact list of people, which can include one or more people along with contact information (e.g., phone number, email address, etc.) for each person. For example, the user interface and display 218 can list a manager of a building in which the deployment station 200 is located as the point of contact for obtaining an access code for this particular deployment station 200.

In some embodiments, the application 1100 includes a fourth button 1108 (e.g., a touch screen button) to return an emergency medical treatment and guidance apparatus 104. For example, when a user presses the fourth button 1108 on the user interface and display 218, the user interface and display 218 can request additional information from the user (e.g., via an interactive query) about which version of the emergency medical treatment and guidance apparatus 104 the user is returning (e.g., emergency medical treatment and guidance apparatus 500, 600, or 700). In some examples, the deployment station 200 will request the user leave the returned emergency medical treatment and guidance apparatus 104 near the deployment station 200 (e.g., on the floor) to complete the return process.

However, in other examples where a return capability is included in the deployment station, e.g., deployment stations 800 and 1000, the deployment station can proceed further. For example, the user interface and display 930 of the deployment station 800 can provide a notification to the user to insert the returned/used emergency medical treatment and guidance apparatus 104 into the return chute 802 of the deployment station 800. In this scenario, when the user inserts the returned/used emergency medical treatment and guidance apparatus 104 into the return chute 802, the process 900 begins as described with reference to FIG. 9. As another example, the user interface and display 1014 of the deployment station 1000 can determine at least one storage section 1002 of the multiple storage sections 1002A, 1002B, 1002C, 1002D, 1002E,1002F that is empty. For example, the deployment station 1000 can query the received signals/information from an RFID reader 812, which in this illustrated embodiment is located at the access door 808, to determine that a sixth one of the storage sections 1002F is empty (the sixth storage section 1002F is shown in FIG. 10). The deployment station 1000 can then unlock the access door 1004 of the empty storage section 1002F so the user can place the returned/used emergency medical treatment and guidance apparatus 104 into the empty storage section 1002F. Then the user can close the access door 1004 of the sixth one of the storage stations 1002F to complete the return process. In some examples, the deployment station 1000 locks the access door 1004 of the sixth one of the storage stations 1002F once the access door 1004 of the sixth one of the storage stations 1002F is closed (e.g., as detected by a contact sensor) to prevent stealing of the returned/used emergency medical treatment and guidance apparatus 104.

In some examples, once the access door 1004 of the sixth one of the storage stations 1002F is closed and locked, the deployment station 1000 identifies the returned/used emergency medical treatment and guidance apparatus 104 (e.g., via the RFID tag of the returned/used emergency medical treatment and guidance apparatus 104) that the user has placed into the sixth one of the storage sections 1002F. In some examples, identifying information about the returned/used emergency medical treatment and guidance apparatus 104 is transmitted to the server and stored in the database. For example, the server 120 of the readiness management system 100 queries the database and determines that a user has returned an emergency medical treatment and guidance apparatus 104 at the deployment station 1000 and transmits a notification to inspectors via one or more of the readiness management devices 130 to attend to the deployment station 1000 and pick up the returned/used emergency medical treatment and guidance apparatus 104.

In some embodiments, the application 1100 includes a fifth button 1110 (e.g., a touch screen button) to return a defibrillator 106. For example, when a user presses the fifth button 1110 on the user interface and display 218, the same process above as described with reference to returning the emergency medical treatment and guidance apparatus 104 is performed by the deployment station 200.

FIGS. 12A-12D show screenshots of an application 1200 running on a user interface and display of a user's mobile device. For context, the description of FIGS. 12A-12D specifically refer to the mobile device 229 as described with reference to FIGS. 2A-2C, but the application 1200 is operable with any of the mobile devices described herein. In some embodiments, the application 1200 is the same as, or substantially similar to the application 1100 described with reference to FIG. 11 above.

In some embodiments, the application 1200 includes a user registration feature 1214 where the user can create an account and log into the user's account on the mobile device 229. For example, during the registration process, the user can link the user's account with the readiness management system 100 (e.g., by requesting an access code from the inspectors or managers of the readiness management system 100). This can be useful in office settings where employees spend a lot of time. In this scenario, the employee/user can create an account and link their account to the readiness management system of the office so that the application 1200 is already set up when needed during a medical emergency. Once logged in, the user registration feature 1214 shows that the user is logged in, as shown for example in FIGS. 12A-12C where the user is shown logged in as “John.” The application 1200 also provides a logout feature 1216 for the user to log out once the user is logged in, as shown in FIGS. 12A-12C.

In some embodiments, the application 1200 includes a first button 1202 (e.g., a touch screen button) to call emergency services (e.g., 911). For example, when a user presses the first button 1202 on the user interface and display of the mobile device 229, the mobile device 229 places a call to emergency services using a transceiver of the mobile device 229. This functionality is substantially the same as the functions of the first button 1102 described with reference to FIG. 11 above.

In some embodiments, the application 1200 includes a second button 1204 (e.g., a touch screen button) to determine a medical need of a patient. For example, when a user presses the second button 1204 on the user interface and display of the mobile device 229, the user interface and display provides an interactive query to determine the medical need of the patient and to recommend an emergency medical treatment and guidance apparatus 104 and/or a defibrillator 106 based on this determined medical need. This functionality is substantially the same as the functions of the second button 1104 described with reference to FIG. 11 above.

In some embodiments, the application 1200 includes third button 1206 (e.g., a touch screen button) to scan a machine-readable code to retrieve identification and/or readiness information of a particular deployment station, emergency medical treatment and guidance apparatus, and/or defibrillator based on the machine-readable code of the particular device/item. For example, when a user presses the third button 1206 on the user interface and display of the mobile device 229, the camera of the mobile device 229 is turned on (if not already on) and the user interface and display requests the user to capture an image of a machine-readable code on a deployment station, an emergency medical treatment and guidance apparatus, or a defibrillator. Once the image is captured, the mobile device 229 decodes the machine-readable code and the mobile device 229 displays the decoded information of the machine-readable code. In examples where the encoded information is a hyperlink, the mobile device 229 is directed to an address within the database to retrieve identifying information and/or readiness information of the particular device scanned and this information is displayed on the user interface of the mobile device 229.

For example, the information displayed can include complete test results (e.g., self-diagnostic test results), battery charge levels for each battery of the devices within the deployment station (e.g., as determined from the battery sensors 274), storage temperatures and humidity (e.g., as determined from the temperature sensors 242 and humidity sensors 246), average and daily highs and lows for temperatures and humidity (e.g., as determined by the controller 203 processing the measured temperature and humidity values), detailed test results of all circuit boards for each electronic apparatus (e.g., as determined via self-evaluation checks), among others. In some examples, the information displayed includes a status, quantity, lot number, expiration date of the medical supplies within each emergency medical treatment and guidance apparatus 104 of the deployment station 200. In some examples, the information displayed includes an expiration date for each electrode of each defibrillator 106 of the deployment station 200. In some examples, the information displayed includes expected inventory, last used dates, last inspection dates, expiration dates of pharmaceuticals, expiration dates of batteries, as determined from a query with a server (e.g., the server 120) and/or from the communicated readiness information from the respective devices directly.

In some embodiments, the application 1200 includes a fourth button 1208 (e.g., a touch screen button) to return an emergency medical treatment and guidance apparatus. For example, when a user presses the fourth button 1208 on the user interface and display of the mobile device 229, the user interface and display requests that the user identify the emergency medical treatment and guidance apparatus being returned. For example, the user interface and display can present instructions to scan a machine-readable code of the emergency medical treatment and guidance apparatus and find a deployment station where the user can return the identified emergency medical treatment and guidance apparatus. Further details are described with reference to FIG. 12B.

FIG. 12B shows the user interface and display of the mobile device 229 displaying a fifth button 1220 with an instruction to “scan a machine-readable code on the device to be returned.” In this example, when the user presses the fourth button 1208 of the user interface and display, the fifth button 1220 is displayed. When the user presses the fifth button 1220, the mobile device 229 turns the camera on (if the camera is not already on), and, at the direction of the user, the camera images the machine-readable code on the emergency medical treatment and guidance apparatus to be returned. Subsequently, the mobile device 229 decodes the information encoded in the machine-readable code and identifies at least a type of the emergency medical treatment and guidance apparatus (e.g., emergency medical treatment and guidance apparatus 500, 600, or 700).

Once the mobile device 229 identifies at least the type of the emergency medical treatment and guidance apparatus, the deployment station where user should return the emergency medical treatment and guidance apparatus of that particular type is determined. For example, the mobile device 229 can communicate the identified type of the emergency medical treatment and guidance apparatus to the server 120, which searches the database for any deployment stations within the readiness management system 100. In some embodiments, the server 120 gives preference to deployment stations with a return chute for returning used emergency medical treatment and guidance apparatuses (e.g., deployment station 800). In some embodiments, the server 120 gives preference to deployment stations with at least one empty storage section for returning used emergency medical treatment and guidance apparatuses (e.g., at least one empty storage section 1002 of the deployment station 1000). In some embodiments, the server 120 sorts the results of the search to give preference to deployment stations closest to the current location of the mobile device 229 (e.g., as determined using a GPS receiver within the mobile device 229 and communicated to the server 120). The server 120 communicates to the mobile device 229 the determined deployment station where user should return the emergency medical treatment and guidance apparatus of that particular type.

FIG. 12C shows the user interface and display of the mobile device 229 displaying instructions 1240 to the user indicating where the user should go to return the emergency medical treatment and guidance apparatus. In some embodiments, the user interface and display includes a sixth button 1242 to, when pressed, cause the mobile device 229 to provide directions from the user's current location to the location of the deployment station. In some embodiments, the user interface and display includes a seventh button 1244 for indicating that the user would like to return the emergency medical treatment and guidance apparatus elsewhere (e.g., because of accessibility constraints [e.g., stairs, long distance, among others]). For example, when the user presses the seventh button 1244, the user interface and display of the mobile device 229 shows a map of all available deployment stations (e.g., similar to the screenshot shown in FIG. 12D and described below) where the user could return the emergency medical treatment and guidance apparatus. In this scenario, the user could press on a particular deployment station of the available deployment stations and the determined deployment station is changed to the selected deployment station. The available deployment stations can be based on the current location of the mobile device 229 such that the available deployment stations are, for example, all the deployment stations at the same address as the mobile device's location, all the deployment stations in a same building as the emergency medical treatment and guidance apparatus in the instructions 1240, all the deployment stations on a same floor as the emergency medical treatment and guidance apparatus in the instructions 1240, or all the deployment stations within a certain predetermined distance of the emergency medical treatment and guidance apparatus in the instructions 1240.

In some embodiments, the application 1200 includes an eighth button 1210 (e.g., a touch screen button) to return a defibrillator. For example, when a user presses the eighth button 1208 on the user interface and display of the mobile device 229, the mobile device 229 performs a substantially similar process as described above for returning an emergency medical treatment and guidance apparatus.

In some embodiments, the application 1200 includes a ninth button 1212 (e.g., a touch screen button) to find nearby deployment stations. For example, when a user presses the ninth button 1212 on the user interface and display of the mobile device 229, the mobile device 229 queries the database to determine all deployment stations within the readiness management system 100. For example, the mobile device 229 can query the database by transmitting a request to the server 120 to find all deployment stations within a building (e.g., a building where the mobile device 229 is located) or a floor of a building (e.g., a building where the mobile device 229 is located), and the server 120 can then transmit the determined deployment station(s) to the mobile device 229 for display to the user. Such functionality can be helpful during a medical emergency since the user may not know ahead of time where the closest deployment station is located.

For example, FIG. 12D shows the application 1200 displaying a map 1270 of a building 1280 with multiple deployment stations A, B, C in proximity to a location of the mobile device 229 (e.g., denoted as “YOU”). For example, the locations of each of deployment stations A, B, C are stored in the database and the location of the mobile device 229 determines a location of the mobile device 229 based on a GPS signal of the mobile device 229.

In some embodiments, the mobile device 229 also queries the database (e.g., by transmitting a request to the server 120) to retrieve readiness information about each deployment station A, B, C shown in the map 1270 and also the readiness information about each emergency medical treatment and guidance apparatus and/or defibrillator within the deployment stations A, B, C shown in the map 1270. For example, the application 1200 indicates the readiness of each deployment station using a “check” indicator 1282 or an “x” indicator 1284 as shown in FIG. 12D. Generally, the “check” is associated with a “ready” state and the “x” is associated with a “needs inspection” state or state that indicates the deployment station is unfit or unsafe for use. In this scenario, the user 230 is informed (e.g., by the indicators 1282 and 1284) which deployment stations and/or devices stored therein are fully stocked and ready for use. In this way, the user 230 can proceed to one of the deployment stations that are ready (e.g., A or C). Other indicators may be employed, for example, indicators that provide further status information regarding whether devices of the deployment station(s) are in need of servicing and also whether they are fit for use or unfit for use.

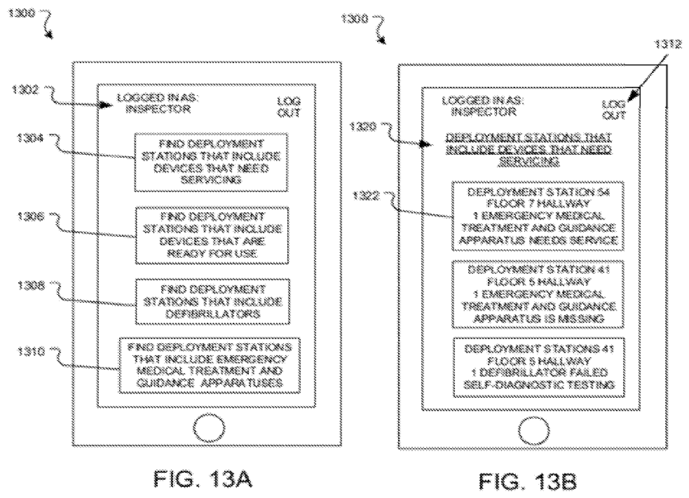

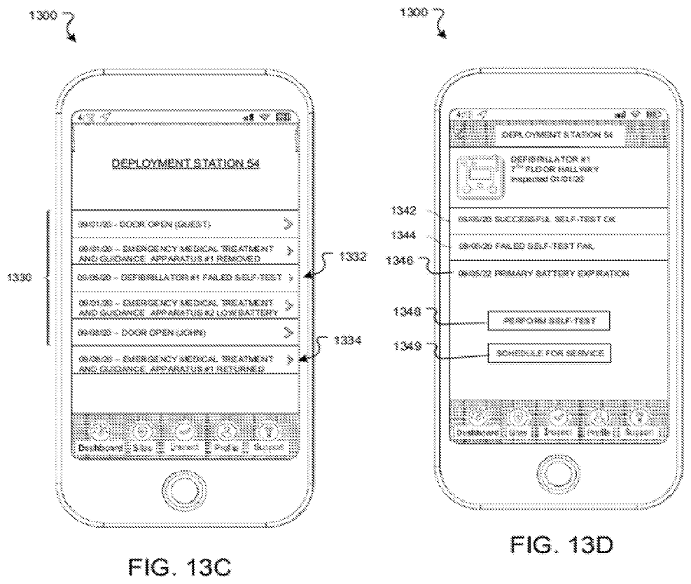

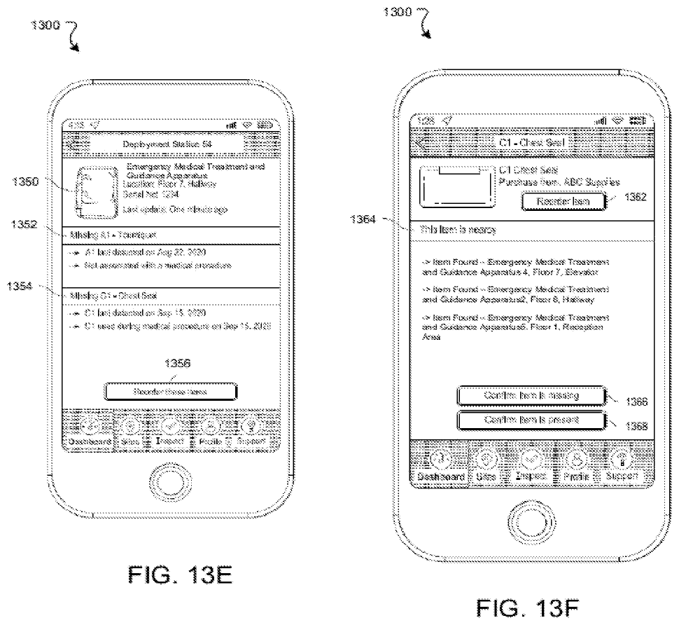

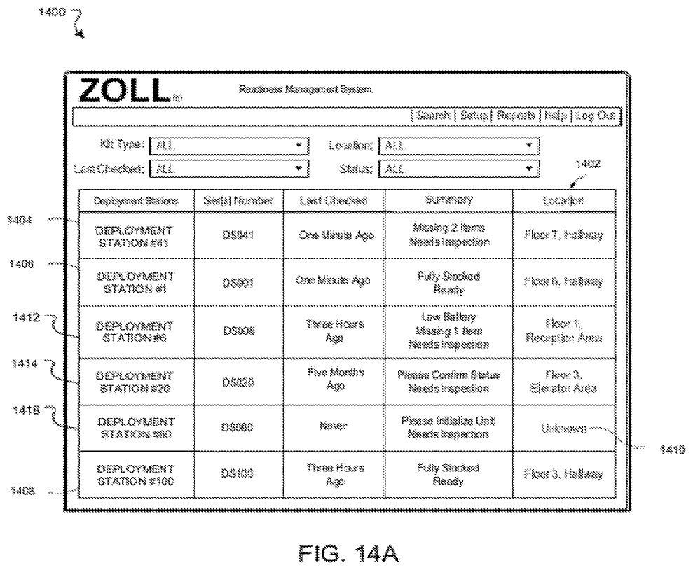

FIGS. 13A-13F show screenshots of an application 1300 on an inspector's mobile device (e.g., a smartphone, a tablet, a smartwatch, among others) as part of the readiness management system 100. For context, the description of FIGS. 13A-13F specifically refer to the mobile device 229 as described with reference to FIGS. 2A-2C, but the application 1300 is operable with any of the mobile devices described herein. In some embodiments, the application 1300 is the same as, or substantially similar to the application 1300 described with reference to FIG. 11 above. In some embodiments, applications 1200 and 1300 are included in a single application.

Referring to FIG. 13A, in some embodiments, the application 1300 includes a user registration feature 1302 where the inspector can create an account and log into the inspectors' account on the mobile device 229. For example, during the registration process, the inspector can link the inspector's account with the readiness management system 100 and specify that the account is a management account (e.g., by providing an access code of the readiness management system 100). Once logged in, the user registration feature 1302 shows that the user is logged in, as shown for example in FIGS. 13A and 13B where the user is shown logged in as “Inspector.” The application 1300 also provides a logout feature 1312 for the user to log out once the user is logged in, as shown in FIGS. 13A and 13B.

In some embodiments, the application 1300 includes a first button 1304 (e.g., a touch screen button) to find deployment stations that include devices that need inspection. For example, when a user presses the first button 1304 on the user interface and display, the mobile device 229 queries the database by transmitting a request to the server 120 to determine all deployment stations within the readiness management system 100 that include at least one device (e.g., an emergency medical treatment and guidance apparatus and/or a defibrillator) that needs inspection. In turn, the mobile device 229 presents a map of deployment stations with devices that need inspection (not shown) as determined by the server 120 and transmitted to the mobile device 229. In this way, the inspector can go to the deployment stations with devices that need inspection while not wasting his/her time on the deployment stations where all devices are ready for use. In some embodiments, when a user presses the first button 1304 on the user interface and display, the mobile device 229 presents the information shown in FIG. 13B described below.

In some embodiments, the application 1300 includes a second button 1306 (e.g., a touch screen button) to find deployment stations that include devices that are ready for use. For example, when a user presses the button 1306 on the user interface and display, the mobile device 229 queries the database by transmitting a request to the server 120 to determine all deployment stations within the readiness management system 100 that include at least one device (e.g., an emergency medical treatment and guidance apparatus and/or a defibrillator) that is ready for use. In turn, the mobile device 229 presents a map of deployment stations that include at least one device that is ready for use (not shown) as determined by the server 120 and transmitted to the mobile device 229, to ensure that medical devices that are ready for use are geographically well distributed and as accessible as possible. In this way, the inspector can go to service the particular deployment stations that do not have at least one device that is ready, so that in the event of a medical emergency, the most deployment stations are available with devices that are ready for use.

In some embodiments, the application 1300 includes a third button 1308 (e.g., a touch screen button) to find deployment stations that include defibrillators. For example, when a user presses the third button 1306 on the user interface and display, the mobile device 229 queries the database by transmitting a request to the server 120 to determine all deployment stations within the readiness management system 100 that include at least one defibrillator. In turn, the mobile device 229 presents a map of deployment stations that include at least one defibrillator (not shown) as determined by the server 120 and transmitted to the mobile device 229. In this way, the inspector can go to the particular deployment stations with at least one defibrillator.

In some embodiments, the application 1300 includes a fourth button 1310 (e.g., a touch screen button) to find deployment stations that include emergency medical treatment and guidance apparatuses. For example, when a user presses the fourth button 1310 on the user interface and display, the mobile device 229 queries the database by transmitting a request to the server 120 to determine all deployment stations within the readiness management system 100 that include at least one emergency medical treatment and guidance apparatus. In turn, the mobile device 229 presents a map of deployment stations that include at least one emergency medical treatment and guidance apparatus (not shown) as determined by the server 120 and transmitted to the mobile device 229. In this way, the inspector can go to the particular deployment stations with at least one emergency medical treatment and guidance apparatus.

Referring to FIG. 13B, in some embodiments, when a user presses the first button 1304 on the user interface and display, the mobile device 229 presents a list 1320 of all deployment stations that include devices that need inspection as determined by the server 120 and transmitted to the mobile device 229. In some embodiments, the list 1320 of deployment stations include a name of the deployment station, a location of the deployment station, and a summary indicating why the deployment station requires attention. In this example, the list 1320 indicates that deployment station 54 is located on the 7^{th} floor hallway and includes one emergency medical treatment and guidance apparatus that needs inspection. In some embodiments, each result of the list 1320 is a button 1322 that the user can press to obtain more information about the deployment station. For example, when a user presses a button 1322 in the list 1320, the user interface and display of the mobile device 229 can present information as shown in FIG. 13C and described below.

The list 1320 also indicates that deployment station 41 is located on the 5^{th} floor hallway and includes a missing emergency medical treatment and guidance apparatus. The list 1320 also indicates that deployment station 41 also includes a defibrillator that has failed self-diagnostic testing.

FIG. 13C shows the application 1300 after the user has pressed the top button 1322 in the list 1320 described with reference to FIG. 13B above. The user interface and display of the mobile device 229 displays a list 1330 of events of the deployment station (deployment station 54 located on the 7^{th} floor hallway). In some embodiments, each result of the list 1330 is a button that the user can press to display additional details about the event. In the example shown, the list 1330 includes an indication that an access door of deployment station 54 located on the 7^{th} floor hallway was opened by a guest (e.g., someone who obtained a guest access code from management), an indication that emergency medical treatment and guidance apparatus #1 was removed from deployment station 54, an indication that defibrillator #1 in deployment station 54 failed a self-evaluation test, an indication that an ECG sensor in deployment station 54 has a problem (e.g., via a signal generator on-board an ECG circuit of the ECG sensor), an indication that an access door of deployment station 54 was opened by a user named “John,” and an indication that emergency medical treatment and guidance apparatus #1 of deployment station 54 was returned. For example, when a user presses a third button 1332 in the list 1330 representing the indication that defibrillator #1 of deployment station 54 failed a self-evaluation test, the user interface and display of the mobile device 229 presents the information shown in FIG. 13D, and when a user presses a last button 1334 in the list 1330 representing the indication that emergency medical treatment and guidance apparatus #1 of deployment station 54 has been returned, the user interface and display of the mobile device 229 presents the information shown in FIG. 13E described below.

FIG. 13D shows the application 1300 after the user has pressed the third button 1332 in the list 1330 described with reference to FIG. 13C above. The user interface and display of the mobile device 229 displays a list of events of defibrillator #1 of deployment station 54. In the example shown, the list of events of defibrillator #1 includes an indication 1342 that the defibrillator #1 passed a self-evaluation test on Mar. 5, 2020 and includes an indication 1344 that the defibrillator #1 failed a self-evaluation test on Sep. 5, 2020. The list also includes an indication 1346 that a primary battery of defibrillator #1 has expired. The user interface and display includes a fifth button 1348 to perform a self-evaluation test of defibrillator #1. For example, when the user presses the fifth button 1348, the mobile device 229 transmits a request to the defibrillator #1 over the readiness management system 100 to perform a self-evaluation test. The user interface and display includes a sixth button 1349 to schedule service for defibrillator #1. For example, when the user presses the sixth button 1349, the mobile device 229 transmits a request to the server 120 to schedule service to repair defibrillator #1.

FIG. 13E shows the application 1300 after the user has pressed the last button 1334 in the list 1330 described with reference to FIG. 13C above. The user interface and display of the mobile device 229 displays a list of events of the emergency medical treatment and guidance apparatus #1 in deployment station 54 with the last known location, serial number, and time since last update. The application 1300 indicates that the A1 Tourniquet 1352 is missing from the emergency medical treatment and guidance apparatus #1 and provides details that the A1 Tourniquet was last detected (e.g., confirmed via inspector entry, detected by one or more RFID readers of the emergency medical treatment and guidance apparatus #1, detected by image processing (e.g., by a controller or a camera of deployment station 54 processing images gathered by the camera), or inferred from a summary (status) report sent from the mobile device 229) on Aug. 22, 2020. The application 1300 indicates that the missing A1 Tourniquet was not associated with a medical procedure (e.g., the server 120 queried the summary (status) reports associated with emergency medical treatment and guidance apparatus #1 for an instance where a Tourniquet was instructed to be used in a medical procedure). Such an indication means that the inspector should physically confirm that the Tourniquet is in fact missing since it may have been stolen or there may be a malfunction with the item and/or the emergency medical treatment and guidance apparatus #1.

Similarly, the application 1300 shows that the C1 Chest Seal 1354 is also missing from emergency medical treatment and guidance apparatus #1 and was last detected on Sep. 15, 2020. Application 1300 indicates that the C1 Chest Seal was used in a medical procedure on Sep. 15, 2020. As described above, the server 120 can query the status reports associated with emergency medical treatment and guidance apparatus #1 to make this determination. In this scenario, the last detected date corresponds with the date when the C1 Chest Seal was used in a medical procedure.

The application 1300 also includes a reorder button 1356 to reorder the missing items shown above, namely the A1 Tourniquet and the C1 Chest Seal. When an inspector clicks the reorder button 1356 a request to reorder one Tourniquet and one Chest Seal is sent from the mobile device 229 to a supplier with a request to deliver the one Tourniquet and the one Chest Seal to the location of emergency medical treatment and guidance apparatus #1. In this scenario, the “Floor 7, Hallway” will be printed on the shipping label such that when the package is received by shipping and receiving, they will deliver the items to the approximate location of emergency medical treatment and guidance apparatus #1. Rather than sending the reorder request directly to the appropriate supplier(s), the mobile device 229 can send the reorder request to the server 120, which in turn sends the reorder request to the appropriate supplier(s). The database can store appropriate suppliers for each possible missing item, which may allow for easier updating of suppliers than if every application on every mobile device has to be updated any time a supplier for any medical item is added, edited, or removed.

FIG. 13F shows a screenshot with additional details of the C1 Chest Seal from FIG. 13E. The additional details can be invoked when a user clicks on the C1 Chest Seal indication 1354 shown in FIG. 13E. A reorder button 1362 enables the inspector to reorder the C1 Chest Seal, similar to that discussed above regarding the reorder button 1356 of FIG. 13E. Furthermore, the additional details show that other C1 Chest Seals are nearby 1364. In this scenario, application 1300 indicates that additional C1 Chest Seals can be found in emergency medical treatment and guidance apparatus #4, #2, and #5. The respective locations of these kits are also indicated. In some examples, this detail screen indicates all stock of C1 Chest Seals including C1 Chest Seals that are in a stock room. Such information can aid the inspector in determining whether to purchase more or move supplies from other kits if necessary.

The application 1300 includes a missing confirmation button 1366 on the additional details screen that, when pressed, stores an entry in the database (e.g., by the mobile device 229 transmitting a request to the server 120 for data to be stored in the database) that the item was confirmed missing by the identification of the inspector (e.g., known via a log-in to the application, etc.) on today's date. Similarly, the application 1300 on the additional details screen includes a present confirmation button 1368 that, when pressed, stores an entry in the database (e.g., by the mobile device 229 transmitting a request to the server 120 for data to be stored in the database) that the item was confirmed present by the identification of the inspector on today's date. Upon pressing either the missing confirmation button 1366 or the present confirmation button 1368, the inspector will be asked to enter any notes to be stored in the database (e.g., by the mobile device 229 transmitting a request to the server 120 for the notes to be stored in the database).

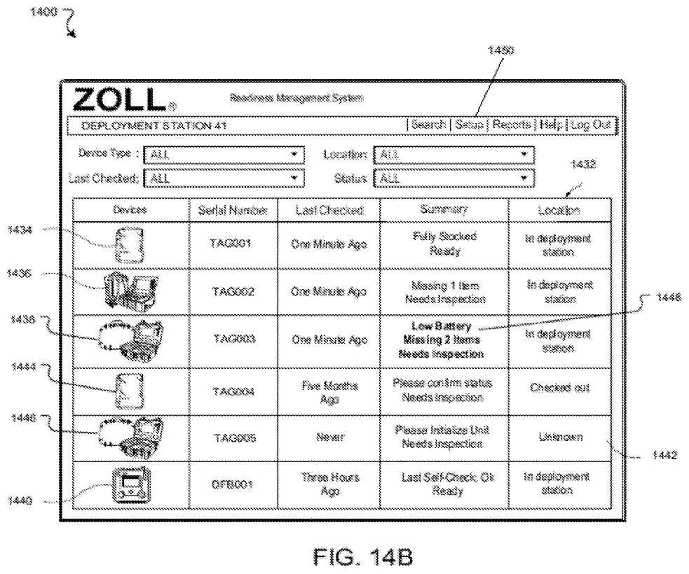

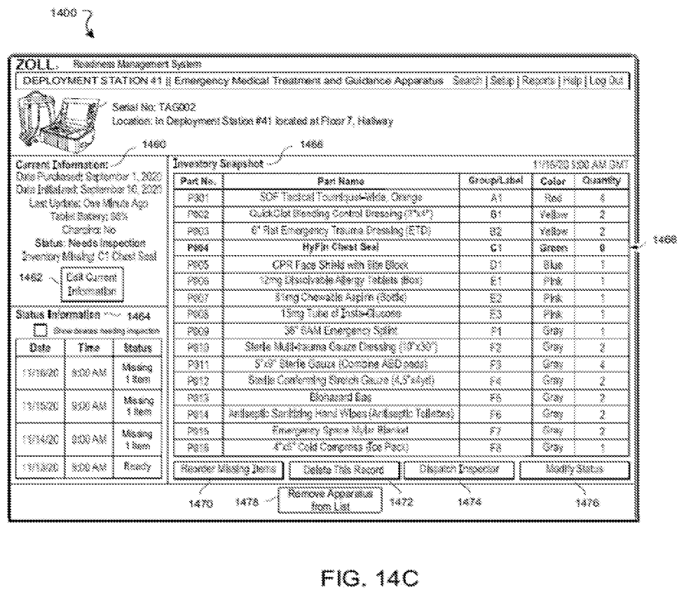

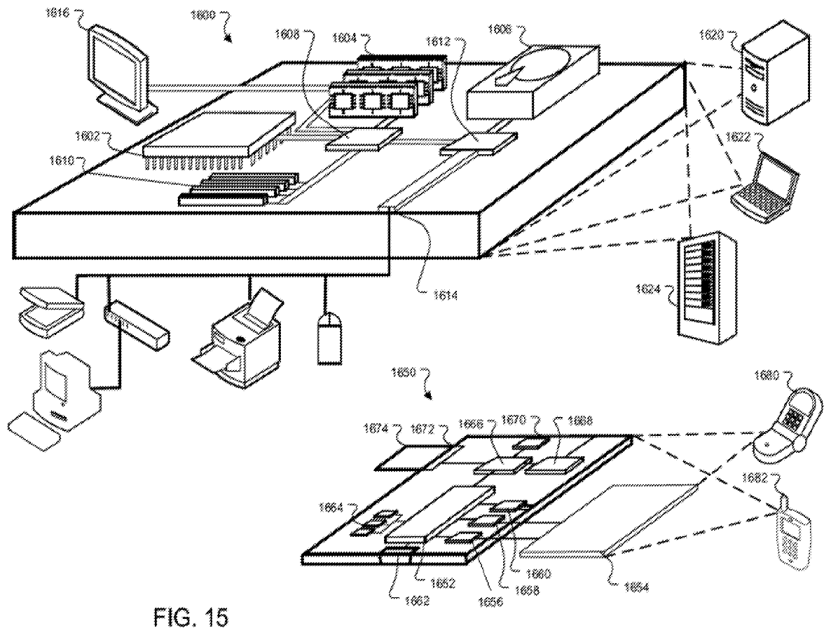

FIGS. 14A-14C show screenshots of an application on an inspector's computational device as part of the readiness management system 100. For example, the computational device can be any of the readiness management devices 130 described with reference to FIG. 1 or the computing devices as described with reference to FIG. 15 for inventory management of one of more emergency medical treatment and guidance apparatuses and defibrillators.

In some embodiments, the application 1400 is similar to application 1300 described with reference to FIGS. 13A-13F above. In some examples, the application 1300 is intended to be performed by an inspector in a mobile setting (e.g., as the inspector travels to and from each deployment station to confirm inventory and check status). Conversely, the application 1400 is intended to be performed by an inspector in a non-mobile setting (e.g., from a desk). However, the application 1400 can be performed in a mobile setting and the application 1300 can be performed in a non-mobile setting. In other words, either of these apps (applications) 1300, 1400 or a combination of their features may be performed by an inspector or person managing the inventory from any location as long as access to the database is achieved.

In some embodiments, the application 1400 includes a list 1402 of deployment stations (e.g., any of the deployment stations described herein) within a readiness management system such as the readiness management system 100 of FIG. 1. In some examples, any of the information stored in a database of a server (e.g., the database of the server 120 of the readiness management system 100) may be queried and shown in the list 1402. In the example shown in FIG. 14A, a first row 1404 includes results for deployment station #41 including a serial number of DS041, a date/time when deployment station #41 was last checked of “One Minute Ago” (e.g., as determined automatically via sensor(s) within deployment station #41), a summary of the readiness status of deployment station #41 of “Missing 2 Items” and “Needs Inspection,” and a location of deployment station #41 of “Floor 7, Hallway.” In this example, the summary information indicates both an overall readiness (e.g., “Needs Inspection”) and readiness information (e.g., “Missing 2 Items”).

In this example, the summary information in the first row 1404 of the list 1402 indicates that at least two medical items are missing from within one or more devices within deployment station #41 (e.g., one device can be missing two items or two devices can be missing one item each). Since deployment station #41 likely has more than one device within it, the summary of the readiness status can represent a worst scenario status of all readiness statuses of the devices within the deployment station. For example, if deployment station #41 has three devices within deployment station #41, two devices may be ready for use, but one may be missing two items. In this way, the application 1400 is configured to present the worst scenario readiness for all devices within a particular deployment station.

In some embodiments, the readiness management system 100 determines the device in most need of servicing by determining which device is missing the most medical items (e.g., the server compares the number of missing items among all devices within a particular deployment station and determines the device with the most missing items). In some embodiments, the readiness management system 100 determines the device in most need of servicing by determining which devices have the greatest number of issues (e.g., low battery, missing items, expired items, etc.). In some embodiments, the readiness management system 100 determines the device in most need of servicing by determining which devices have the greatest threat to a user (e.g., by prioritizing devices with expired pharmaceuticals that could cause harm if injected into a person, etc.).

A second row 1406 of the list 1402 includes results for deployment station #1 including a serial number of DSO01, a date/time last checked of “One Minute Ago,” a summary of the readiness status of “Fully Stocked” and “Ready,” and a location of “Floor 6, Hallway.” In this example, the readiness status indicates both an overall readiness of all the devices within the deployment station #1 (e.g., “Ready”) and readiness information about the each of the device within the deployment station #1 (e.g., “Fully Stocked).

A third row 1412 of the list 1402 includes results for deployment station #6 including a serial number of DSO06, a date/time last checked of “Three Hours Ago,” a summary of the readiness status of deployment station #6 of “Low Battery,” “Missing 1 Item,” and “Needs Inspection,” and a location of deployment station #6 of “Floor 1, Reception Area.” In this example, the summary information indicates both an overall readiness (e.g., “Needs Inspection”) and multiple readiness information (e.g., “Low Battery” and “Missing 2 Items”). In this example, the summary information indicates that at least two medical items are missing from within one or more devices within deployment station #6 (e.g., one device can be missing two items or two devices can be missing one item each). Similarly, deployment station #6 as a whole requires inspection.

A fourth row 1414 of the list 1402 includes results for deployment station #20 including a serial number of DSO20, a date/time last checked of “Five Months Ago,” a summary of the readiness status of deployment station #20 of “Please Confirm Status” and “Needs Inspection,” and a location of deployment station #20 of “Floor 3, Elevator Area.” In this example, the summary information indicates that the status should be confirmed by an inspector. For example, the database may have received conflicting readiness information (e.g., received an indication that a device within deployment station #20 is missing a medical supply followed by an indication that that particular medical supply is no longer missing. In some examples, the server periodically queries the database to identify conflicting information by comparing the changes in readiness with associated times of the readiness changes.

A fifth row 1416 of the list 1402 includes results for deployment station #60 including a serial number of DSO60, a date/time last checked of “Never,” a summary of the readiness status of deployment station #60 of “Please Initialize Unit” and “Needs Inspection,” and a location 1410 of deployment station #60 of “Unknown.” In this example, deployment station #60 is currently awaiting initialization (e.g., the deployment station may still need to be connected to the readiness management system 100). In this example, because initialization is needed, deployment station #60 has not yet reported in a status update (e.g., last checked “Never”).

A sixth row 1408 of the list 1402 includes results for deployment station #100 including a serial number of DS100, a date/time last checked of “Three Hours Ago,” a summary of the readiness status of “Fully Stocked” and “Ready,” and a location of deployment station #100 of “Floor 3, Hallway.” In this example, the readiness status indicates both an overall readiness of all the devices within deployment station #100 (e.g., “Ready”) and readiness information about the each of the device within deployment station #100 (e.g., “Fully Stocked”).

In some embodiments, each row of the list 1402 is a button so the user can click on a particular row (e.g., by touch, using a mouse, etc.) and the application 1400 provides additional details about that particular deployment station. For example, when a user clicks on the first row 1404, the application 1400 presents additional details of deployment station #41 on a user interface of the inspector's computational device as shown in FIG. 14B. This is particularly useful when medical supplies are missing and/or the deployment station needs inspection. In this way, the application 1400 allows a user to see exactly which medical supplies are missing. For example, FIG. 14B shows further details of deployment station #41.

FIG. 14B shows a screenshot of the application 1400 after the user clicks on the first row 1404 as described with reference to FIG. 14A above. In this example, details regarding deployment station #41 are presented to the user in a details list 1432. In particular, FIG. 14B shows all emergency medical treatment and guidance apparatuses and all defibrillators within deployment station #41. While not shown in FIG. 14B, fire extinguishers are also included on this details list 1432 in some examples. All of the emergency medical treatment and guidance apparatuses and all of the defibrillators within deployment station #41 are visible in the screenshot of FIG. 14B, but the user may need to scroll down the list 1432 to view all emergency medical treatment and guidance apparatuses, all defibrillators, and all fire extinguishers within a deployment station. For example, FIG. 14B shows that deployment station #41 includes six devices, each device represented by a different row in the details list 1432. In this way, the application 1400 includes a details list 1432 of all devices within deployment station #41 (e.g., any of the emergency medical treatment and guidance apparatus and/or defibrillators described herein and in some examples fire extinguishers) within the readiness management system 100. In some examples, any of the information stored in the database may be queried and shown in the details list 1432.

As noted above with reference to FIG. 14A, the readiness status of deployment station #41 indicates that deployment station #41 is “Missing 2 Items” and “Needs Inspection.” As shown in FIG. 14B, this worst scenario status is caused by the device of a third row 1438 of the details list 1432 having a readiness status 1448 of “Low Battery,” “Missing 2 Items,” and “Needs Inspection.” In some examples, as shown in FIG. 14B, the application 1400 uses a bolded font or a different font color to indicate to the person viewing the application 1400 that the readiness status 1448 is the worst readiness of the deployment station.

Referring to the details list 1432 in general, in some embodiments, each device of the details list 1432 can be associated with an image identifier. For example, a stock image can be used in the “Devices” column of the details list 1432 to indicate a type of the device, e.g., whether the device is the emergency medical treatment and guidance apparatus 500, the emergency medical treatment and guidance apparatus 600, the emergency medical treatment and guidance apparatus 700, or the defibrillator 400. In examples where the device is not one of the emergency medical treatment and guidance apparatus 500, the emergency medical treatment and guidance apparatus 600, the emergency medical treatment and guidance apparatus 700, or the defibrillator 400, a stock image of another device can be used.

For example, a first row 1434 of the details list 1432 includes results for an emergency medical treatment and guidance apparatus (e.g., emergency medical treatment and guidance apparatus 700 in this illustrated embodiment) including an image of the emergency medical treatment and guidance apparatus 700, a serial number of TAG001, a date last checked of “One Minute Ago” (e.g., determined automatically via sensor(s) within deployment station #41 and/or within the emergency medical treatment and guidance apparatus when applicable), a summary of the readiness status of the emergency medical treatment and guidance apparatus of “Fully Stocked” and “Ready,” and a location of “In deployment station.” The first row 1434 of the details list 1432 indicates that the emergency medical treatment and guidance apparatus of the first row 1434 is within deployment station #41 as of one minute ago and is ready for use and is fully stocked. In this scenario, deployment station #41 detected the presence of the device of the first row 1434 within the deployment station one minute ago.

A second row 1436 of the details list 1432 includes results for a different emergency medical treatment and guidance apparatus (e.g., emergency medical treatment and guidance apparatus 600 in this illustrated embodiment). The second row 1436 of the details list 1432 indicates that the emergency medical treatment and guidance apparatus of the second row 1436 has a serial number of TAG002, a date last checked of “One Minute Ago,” a summary of the readiness status of the emergency medical treatment and guidance apparatus of “Missing 1 Item” and “Needs Inspection,” and a location of “In deployment station.” The second row 1436 of the details list 1432 indicates that the emergency medical treatment and guidance apparatus of the second row 1436 is within the deployment station as of one minute ago but is in need of service. The emergency medical treatment and guidance apparatus of the second row 1436 of the details list 1432 is missing one medical item.

A third row 1438 of the details list 1432 includes results for a different emergency medical treatment and guidance apparatus (e.g., emergency medical treatment and guidance apparatus 500 in this illustrated embodiment). The third row 1438 of the details list 1432 indicates that the emergency medical treatment and guidance apparatus of the third row 1438 has a serial number of TAG003, a date last checked of “One Minute Ago,” a summary of the readiness status of the emergency medical treatment and guidance apparatus of “Low Battery,” “Missing 2 Items,” and “Needs Inspection,” and a location of “In deployment station.” The third row 1438 of the details list 1432 indicates that the emergency medical treatment and guidance apparatus of the third row 1438 is within the deployment station as of one minute ago but is in need of service. The emergency medical treatment and guidance apparatus of the third row 1438 of the details list 1432 is missing two medical items and contains a battery in need of charge.

A fourth row 1444 of the details list 1432 includes results for a different emergency medical treatment and guidance apparatus (e.g., emergency medical treatment and guidance apparatus 700 in this illustrated embodiment). The fourth row 1444 of the details list 1432 indicates that the emergency medical treatment and guidance apparatus of the fourth row 1444 has a serial number of TAG004, a date last checked of “Five Months Ago,” a summary of the readiness status of the emergency medical treatment and guidance apparatus of “Please confirm status” and “Needs Inspection,” and a location of “Checked out.” The fourth row 1444 of the details list 1432 indicates that the emergency medical treatment and guidance apparatus of the fourth row 1444 has not been checked within the last five months, needs inspection, and is not within the deployment station. In this example, a user may have checked out the emergency medical treatment and guidance apparatus of the fourth row 1444 five months ago for a medical need but has not returned the emergency medical treatment and guidance apparatus to deployment station #41.

In some embodiments, the readiness management system 100 determines the whether to update the readiness to “Please confirm status” when the particular device has not transmitted a readiness to the database after a period of time has elapsed. In some examples, the period of time can be one week, one month, or three months, etc.

A fifth row 1446 of the details list 1432 includes results for a different emergency medical treatment and guidance apparatus (e.g., emergency medical treatment and guidance apparatus 500 in this illustrated embodiment). The fifth row 1446 of the details list 1432 indicates that the emergency medical treatment and guidance apparatus of the fifth row 1446 has a serial number of TAG005, a date last checked of “Never,” a summary of the readiness status of the emergency medical treatment and guidance apparatus of “Please Initialize Unit,” and “Needs Inspection,” and a location 1442 of “Unknown.” The fifth row 1446 of the details list 1432 indicates that the emergency medical treatment and guidance apparatus of the fifth row 1446 has never been checked, the location of the device is unknown, and is instructing the viewer of the application 1400 to properly set up the device. For example, such a scenario can arise when a device is manually added into the application 1400 by an inspector but the device is not otherwise set up.

A sixth row 1440 of the details list 1432 includes results for a defibrillator (e.g., defibrillator 400 in this illustrated embodiment). The sixth row 1440 indicates that the defibrillator of the sixth row 1440 has a serial number of DFB001, a date last checked of “Three Hours Ago,” a summary of the readiness status of the defibrillator of “Last Self-Check: Ok” and “Ready,” and a location of “In deployment station.” The sixth row 1440 of the details list 1432 indicates that the defibrillator of the sixth row 1440 is within the deployment station as of three hours ago and is ready for use.

As shown in this example of the details list 1432, the frequency of the last checked information can vary amongst the devices within the deployment station. In some examples, this frequency is user determined based on user preferences of the devices and/or the deployment station. For example, the frequency of when the readiness information is determined (e.g., when the device is “last checked”) can be minutely, hourly, daily, etc. and then transmitted by the device and/or deployment station to the database and the details list 1432 updated.

In the example shown in FIG. 14B, each device includes serial number information (or asset tag) that is a manufacturer-assigned identification number that uniquely identifies the device. Each device includes last checked status information that indicates the most recent “check-in” of the device. In some scenarios, the last checked status is provided by an inspector actually confirming the status and in other scenarios, the last checked status represents the most recent time when the device sent status information to the database automatically (e.g., by querying the RFID tags, by sending a summary report of a use of the device etc.) In examples of defibrillators, the last checked date can represent the most recent date a self-diagnostic check was received by the database.

Each device in the details list 1432 includes summary information with readiness information. The summary information indicates whether the emergency medical treatment and guidance apparatus is determined to be ready for use, i.e., ready to be used for a medical emergency.

The ready for use determination may require that the inventory of an emergency medical treatment and guidance apparatus is full (e.g., no medical supplies are determined to be missing (e.g., inferred via imaging processing, inferred via the status report, or inferred via the RFID tags) or no medical supplies are confirmed to be missing (e.g., by the inspector manually)). The ready for use determination may require that each battery associated with the device is above a threshold (e.g., above 20%, above 50%, or above 75%). The specific battery level used in determining whether the device is ready for us is user programmable (e.g., via settings within a “Setup” menu 1450 of the application 1400). In some examples, the battery is the battery of the mobile devices or tablets within the emergency medical treatment and guidance apparatus. In some examples, the battery is the battery of the deployment station.

The ready for use determination may require that the last checked status (e.g., last test data) be within a recent time frame (e.g., within the last 1 month, or within the last 2 months). If the last checked status was received before this time frame, the server determines that the device may not be ready for use and flags it (e.g., via the “needs inspection” indication) for an inspector to check on the status manually. For example, in some scenarios, the battery of the mobile device or tablet has died and status updates are no longer received by the database. In other examples, the device has moved outside of the network and is no longer able to send status updates. In this scenario, the location of the device may be updated to reflect an “Unknown” location.

The ready for use determination may require that a device included in the emergency medical treatment and guidance apparatus pass a self-diagnostic check. For example, in scenarios where the emergency medical treatment and guidance apparatus include a defibrillator with a self-diagnostic capability, the emergency medical treatment and guidance apparatus may report in the status of the self-diagnostic check and the results may be indicated in the “Summary” column.

In some examples, a device needing inspection that is “suitable for use” represents a scenario where a device needing inspection is usable to treat at least some of the medical emergencies that a ready for use device apparatus can treat. In some examples, the “suitable for use” determination is made by the inspector and entered into the database (e.g., entered by the inspector via one or more of the readiness management devices 130 described herein and stored in the database via inclusion in the summary information).

In some examples, the “suitable for use” determination is performed by the emergency medical treatment and guidance apparatus, e.g., via inclusion in the summary information. In some examples, the “suitable for use” indication is presented on the user interfaces of the readiness management devices 130. In some examples, the ready for use determination is based on an emergency medical treatment and guidance apparatus including, one or more of the following conditions: a majority of medical supplies are present, at least particular medical supplies are present (e.g., an A1 Tourniquet and a C1 Chest Seal), and/or a battery is low but not empty (e.g., between 25% and 75%), among others).

Each device includes location information in the details list 1432. The location information may indicate the building identification, a floor, a room, or a hallway location of the device. In some embodiments, each row of the details list 1432 is a button so the user can click the row (e.g., by touch, using a mouse, etc.) and the application 1400 provides additional details about that particular device. For example, when a user clicks on the second row 1436, the application 1400 presents additional details of the emergency medical treatment and guidance apparatus 600 on the user interface as shown in FIG. 14C.

FIG. 14C shows a screenshot of the application 1400 after the user clicks on the second row 1436 of the details list 1432 as described with reference to FIG. 14B above. In this example, details regarding the emergency medical treatment and guidance apparatus 600 of the second row 1436 are presented to the user. The application 1400 includes current information 1460 that includes the date purchased (e.g., the date the emergency medical treatment and guidance apparatus was purchased), the date initialized (e.g., the date the emergency medical treatment and guidance apparatus was initially set-up), the last update date (e.g., the date last checked in FIG. 14B), the battery status (if applicable), whether the mobile device or tablet is charging (if applicable), and whether the emergency medical treatment and guidance apparatus is ready for use (e.g., as shown in the “Summary” column of FIG. 14B). An edit current information button 1462 is provided such that when the edit current information button 1654 is pressed, the information in the current information window 1460 may be modified by the inspector.

In some embodiments, the application 1400 includes status information 1464 that presented a chronological order of status information received from the emergency medical treatment and guidance apparatus. The status information 1464 also includes manual updates from inspectors. The status information 1464 includes a readiness status in accordance with the determination described with reference to the “Summary” column of FIG. 14B.

In some embodiments, the application 1400 includes an inventory snapshot 1466 that includes a list of each part number, part name, group/label identification, color, and quantity of each medical item within the emergency medical treatment and guidance apparatus. In some examples, expiration date information is also included in the inventory snapshot 1466 list. The specific columns of the inventory snapshot 1466 shown can be changed via a settings option within the “Setup” menu 1450.

In some embodiments, the inventory snapshot 1466 emphases a medical item with a determined quantity that is less than an expected quantity. For example, the expected quantity is configurable when setting up the readiness management system and/or anytime via the application (e.g., via the edit current information button 1462). In the example shown, a fourth row 1468 in the inventory snapshot 1466 indicates that the “HyFin Chest Seal” has a quantity of “0” while the emergency medical treatment and guidance apparatus expects a quantity of “1.” As a result, the application 1400 shows the row emphasized in bold font (but different colors or highlighting may also be used) to direct the inspectors attention to the medical item. Referring back to FIG. 14B, the emergency medical treatment and guidance apparatus in the second row 1436 of the details list 1432 shows a “Summary” of “Missing 1 Item” indicative of the missing HyFin Chest Seal.

The application 1400 includes a button (or option) to “Reorder Missing Items” 1470 that, when pressed, transmits a request to purchase 1 HyFin Chest Seal from a supplier. The one HyFin Chest Seal is the only missing inventory item so is the only item reordered in response to the pressing of the “Reorder Missing Items” button 1470, but any other missing items would also be ordered by pressing the “Reorder Missing Items” button 1470. In some examples, the readiness management system can be configured to request the medical item be shipped to the location of the deployment station and/or emergency medical treatment and guidance apparatus itself.

In some embodiments, the application 1400 includes a button (or option) to generate a record (not shown) to generate a report of the missing medical supplies that is then stored in the database for querying by the devices of the readiness management system.

In some embodiments, the application 1400 includes a button (or option) to provide a notification to an inspector (e.g., via a mobile phone) so that the inspector is notified to confirm whether the missing medical item(s) need to be replenished or if the notification can be dismissed (i.e., the medical item(s) do not need to be replenished).

In some embodiments, the application 1400 includes a button (or option) to “Delete this Record” 1472 that removes specific columns from the inventory snapshot 1466 list.

In some embodiments, the application 1400 includes a button (or option) to “Dispatch Inspector” 1474 that transmits a request to inspectors within the network of the readiness management system or inspectors within a radius to the deployment station and/or the emergency medical treatment and guidance apparatus to check on the status of the emergency medical treatment and guidance apparatus. Along with the dispatch request, the readiness management system sends details about the missing items so the inspector is aware what he/she is expected to confirm.

In some embodiments, the application 1400 includes a button (or option) to “Modify Status” 1476 that edits specific columns within the list. For example, if one or more details within the table is wrong, the details can be edited using this option.

In some embodiments, the application 1400 includes a button (or option) to “Remove Apparatus from List” 1478 that removes the entire emergency medical treatment and guidance apparatus from the readiness management system.

FIG. 15 is a diagram of computer systems forming part of the portable emergency medical treatment and guidance apparatuses and/or defibrillators according to some embodiments. For example, a computing device 1600 and a mobile computing device 1650 can be used to implement the techniques previously described. The computing device 1600 is intended to represent various forms of digital computers, including, e.g., laptops, desktops, workstations, personal digital assistants, servers, blade servers, mainframes, and other appropriate computers. The mobile computing device 1650 is intended to represent various forms of mobile devices, including, e.g., personal digital assistants, tablet computing devices, cellular telephones, smartphones, and other similar computing devices. The components shown here, their connections and relationships, and their functions, are meant to be examples only, and are not meant to limit implementations of the techniques described and/or claimed in this document.

The computing device 1600 includes processor 1602, memory 1604, storage device 1606, high-speed interface 1608 connecting to memory 1604 and high-speed expansion ports 1610, and low-speed interface 1612 connecting to a low speed bus 1614 and storage device 1606. Each of these components 1602, 1604, 1606, 1608, 1610, 1612, 1614, is interconnected using various busses, and can be mounted on a common motherboard or in other manners as appropriate. The processor 1602 can process instructions for execution within the computing device 1600, including instructions stored in the memory 1604 or on the storage device 1606 to display graphical data for a GUI on an external input/output device, including, e.g., a display 1616 coupled to the high speed interface 1608. In other implementations, multiple processors and/or multiple busses can be used, as appropriate, along with multiple memories and types of memory. Also, multiple computing devices 1600 can be connected, with each device providing portions of the necessary operations (e.g., as a server bank, a group of blade servers, or a multi-processor system).

The memory 1604 stores data within the computing device 1600. In one implementation, the memory 1604 is a volatile memory unit or units. In another implementation, the memory 1604 is a non-volatile memory unit or units. The memory 1604 also can be another form of computer-readable medium (e.g., a magnetic or optical disk). The memory 1604 may be non-transitory.

The storage device 1606 is capable of providing mass storage for the computing device 1600. In one implementation, the storage device 1606 can be or contain a computer-readable medium (e.g., a floppy disk device, a hard disk device, an optical disk device, or a tape device, a flash memory or other similar solid state memory device, or an array of devices, such as devices in a storage area network or other configurations.) A computer program product can be tangibly embodied in a data carrier. The computer program product also can contain instructions that, when executed, perform one or more methods (e.g., those described above.) The data carrier is a computer- or machine-readable medium, (e.g., the memory 1604, the storage device 1606, memory on the processor 1602, and the like.)

The high-speed controller 1608 manages bandwidth-intensive operations for the computing device 1600, while the low-speed controller 1612 manages lower bandwidth-intensive operations. Such allocation of functions is an example only. In one implementation, the high-speed controller 1608 is coupled to the memory 1604, the display 1616 (e.g., through a graphics processor or accelerator), and to the high-speed expansion ports 1610, which can accept various expansion cards (not shown). In the implementation, the low-speed controller 1612 is coupled to the storage device 1606 and the low-speed expansion port 1614. The low-speed expansion port 1614, which can include various communication ports (e.g., USB, Bluetooth®, Ethernet, wireless Ethernet), can be coupled to one or more input/output devices, (e.g., a keyboard, a pointing device, a scanner, or a networking device including a switch or router, e.g., through a network adapter.)

The computing device 1600 can be implemented in a number of different forms, as shown in the FIG. 15. For example, it can be implemented as standard server 1620, or multiple times in a group of such servers. It also can be implemented as part of a rack server system 1624. In addition or as an alternative, it can be implemented in a personal computer (e.g., a laptop computer 1622). In some examples, components from the computing device 1600 can be combined with other components in a mobile device (not shown), e.g., the mobile computing device 1650. Each of such devices can contain one or more of the computing device 1600, 1650, and an entire system can be made up of multiple computing devices 1600, 1650 communicating with each other.

The mobile computing device 1650 includes processor 1652, memory 1664, an input/output device (e.g., display 1654, communication interface 1666, and transceiver 1668) among other components. The mobile computing device 1650 also can be provided with a storage device, (e.g., a microdrive or other device) to provide additional storage. Each of these components 1652, 1664, 1654, 1666, 1668 is interconnected using various busses, and several of the components can be mounted on a common motherboard or in other manners as appropriate.

The processor 1652 can execute instructions within the mobile computing device 1650, including instructions stored in the memory 1664. The processor 1652 can be implemented as a chipset of chips that include separate and multiple analog and digital processors. The processor 1652 can provide, for example, for coordination of the other components of the mobile computing device 1650, e.g., control of user interfaces, applications run by the mobile computing device 1650, and wireless communication by the mobile computing device 1650.

The processor 1652 can communicate with a user through a control interface 1658 and a display interface 1656 coupled to the display 1654. The display 1654 can be, for example, a TFT LCD (Thin-Film-Transistor Liquid Crystal Display) or an OLED (Organic Light Emitting Diode) display, or other appropriate display technology. The display interface 1656 can comprise appropriate circuitry for driving the display 1654 to present graphical and other data to a user. The control interface 1658 can receive commands from a user and convert them for submission to the processor 1652. In addition, an external interface 1662 can communicate with the processor 1652, so as to enable near area communication of the mobile computing device 1650 with other devices. The external interface 1662 can provide, for example, for wired communication in some implementations, or for wireless communication in other implementations, and multiple interfaces also can be used.

The memory 1664 stores data within the mobile computing device 1650. The memory 1664 can be implemented as one or more of a computer-readable medium or media, a volatile memory unit or units, or a non-volatile memory unit or units. Expansion memory 1674 also can be provided and connected to the mobile computing device 1650 through an expansion interface 1672, which can include, for example, a SIMM (Single In Line Memory Module) card interface. Such expansion memory 1674 can provide extra storage space for the mobile computing device 1650, or also can store applications or other data for the mobile computing device 1650. Specifically, the expansion memory 1674 can include instructions to carry out or supplement the processes described above, and can include secure data also. Thus, for example, the expansion memory 1674 can be provided as a security module for the mobile computing device 1650, and can be programmed with instructions that permit secure use of the mobile computing device 1650. In addition, secure applications can be provided through the SIMM cards, along with additional data, (e.g., placing identifying data on the SIMM card in a non-hackable manner.)

The memory 1664 can include, for example, flash memory and/or NVRAM memory, as discussed below. In one implementation, a computer program product is tangibly embodied in a data carrier. The computer program product contains instructions that, when executed, perform one or more methods, e.g., those described above. The data carrier is a computer- or machine-readable medium (e.g., the memory 1664, the expansion memory 1674, and/or memory on the processor 1652), which can be received, for example, over the transceiver 1668 or the external interface 1662.

The mobile computing device 1650 can communicate wirelessly through a communication interface 1666, which can include digital signal processing circuitry where necessary. The communication interface 1666 can provide for communications under various modes or protocols (e.g., GSM voice calls, SMS, EMS, or MNS messaging, CDMA, TDMA, PDC, WCDMA, CDMA1500, or GPRS, among others.) Such communication can occur, for example, through the radio-frequency transceiver 1668. In addition, short-range communication can occur, e.g., using a Bluetooth®, WiFi, or other such transceiver (not shown). In addition, a GPS (Global Positioning System) receiver module 1670 can provide additional navigation- and location-related wireless data to the mobile computing device 1650, which can be used as appropriate by applications running on the mobile computing device 1650. Sensors and modules such as cameras, microphones, compasses, accelerators (for orientation sensing), etc. may be included in the mobile computing device 1650.

The mobile computing device 1650 also can communicate audibly using an audio codec 1660, which can receive spoken data from a user and convert it to usable digital data. The audio codec 1660 can likewise generate audible sound for a user, (e.g., through a speaker in a handset of the mobile computing device 1650.) Such sound can include sound from voice telephone calls, can include recorded sound (e.g., voice messages, music files, and the like) and also can include sound generated by applications operating on the mobile computing device 1650.

The mobile computing device 1650 can be implemented in a number of different forms, as shown in the FIG. 15. For example, it can be implemented as a cellular telephone 1680. It also can be implemented as part of a smartphone 1682, a personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor. The programmable processor can be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to a computer program product, apparatus and/or device (e.g., magnetic discs, optical disks, memory, Programmable Logic Devices (PLDs)) used to provide machine instructions and/or data to a programmable processor, including a machine-readable medium that receives machine instructions.

To provide for interaction with a user, the systems and techniques described here can be implemented on a computer having a device for displaying data to the user (e.g., a CRT (cathode ray tube) or LCD (liquid crystal display) monitor), and a keyboard and a pointing device (e.g., a mouse or a trackball) by which the user can provide input to the computer. Other kinds of devices can be used to provide for interaction with a user as well; for example, feedback provided to the user can be a form of sensory feedback (e.g., visual feedback, auditory feedback, or tactile feedback); and input from the user can be received in a form, including acoustic, speech, or tactile input.

The systems and techniques described here can be implemented in a computing system that includes a backend component (e.g., as a data server), or that includes a middleware component (e.g., an application server), or that includes a frontend component (e.g., a client computer having a user interface or a Web browser through which a user can interact with an implementation of the systems and techniques described here), or a combination of such back end, middleware, or frontend components. The components of the system can be interconnected by a form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

While the inventive features described herein have been described in terms of a preferred embodiment for achieving the objectives, it will be appreciated by those skilled in the art that variations may be accomplished in view of these teachings without deviating from the spirit or scope of the present disclosure.

What is claimed is:

1. A deployment station for portable emergency medical treatment and guidance apparatuses, the deployment station comprising:
a first storage section for holding at least one emergency medical treatment and guidance apparatus comprising a plurality of medical supplies;
a second storage section for holding at least one defibrillator comprising electronic circuitry to administer electrotherapy; and
a housing containing the first storage section and the second storage section, the housing comprising:
a readiness indicator disposed on an exterior surface of the housing for indicating a readiness of the at least one emergency medical treatment and guidance apparatus and the at least one defibrillator based on obtained readiness information; and
at least one processor and memory mechanically coupled to the housing and communicatively coupled to the readiness indicator, the at least one processor and memory configured for:
obtaining the readiness information of the at least one emergency medical treatment and guidance apparatus and the at least one defibrillator;
determining readiness of the at least one emergency medical treatment and guidance apparatus and the at least one defibrillator based on the obtained readiness information;
updating the readiness indicator based on the determined readiness; and
transmitting a readiness signal representing the readiness of the at least emergency medical treatment and guidance apparatus and the at least one defibrillator to a server for maintaining a database of the readiness of the at least one emergency medical treatment and guidance apparatus and the at least one defibrillator.

2. The deployment station of claim 1, wherein the housing further comprises at least one sensor for obtaining the readiness information of the at least one emergency medical treatment and guidance apparatus and the at least one defibrillator, the at least one processor and memory configured for receiving a signal from the at least one sensor.

3. The deployment station of claim 2, wherein the at least one processor and memory is configured for identifying the at least one emergency medical treatment and guidance and the at least one defibrillator based on identification information obtained from the at least one sensor.

4. The deployment station of claim 3, wherein the at least one sensor comprises a radio frequency identification (RFID) reader and each of the at least one emergency medical treatment and guidance apparatuses and each of the at least one defibrillators comprises a respective unique RFID tag readable by the RFID reader, wherein the identification information comprises a serial number corresponding to the at least one emergency medical treatment and guidance apparatus and/or the at least one defibrillator, and the readiness information comprises an expiration date corresponding to the at least one emergency medical treatment and guidance apparatus and/or the at least one defibrillator.

5. The deployment station of claim 4, wherein the expiration date corresponding to the at least one defibrillator comprises an expiration date of electrodes for administering electrotherapy.

6. The deployment station of claim 4, wherein the expiration date corresponding to the at least one emergency medical treatment and guidance apparatus comprises an expiration date of at least one of the plurality of medical supplies.

7. The deployment station of claim 1, wherein determining readiness of the at least one emergency medical treatment and guidance apparatus and the at least one defibrillator comprises:
determining whether a battery of the at least one emergency medical treatment and guidance apparatus or the at least one defibrillator has a battery charge above a pre-determined threshold,
wherein the battery comprises a battery of a touch screen user interface of the at least one emergency medical treatment and guidance apparatus.

8. The deployment station of claim 1, wherein determining readiness of the at least one emergency medical treatment and guidance apparatus and the at least one defibrillator comprises:
determining how an inventory of medical supplies within the at least one emergency medical treatment and guidance apparatus compares with an expected inventory representing a fully stocked emergency medical treatment and guidance apparatus, the expected inventory being retrieved from the database.

9. The deployment station of claim 8, wherein determining how the inventory of the medical supplies within the at least one emergency medical treatment and guidance apparatus compares with the expected inventory representing a fully stocked emergency medical treatment and guidance apparatus comprises determining that the inventory of the medical supplies matches the expected inventory.

10. The deployment station of claim 8, wherein determining how the inventory of the medical supplies within the at least one emergency medical treatment and guidance apparatus compares with the expected inventory representing a fully stocked emergency medical treatment and guidance apparatus comprises determining that at least a portion of the inventory of the medical supplies is missing.

11. The deployment station of claim 1, wherein determining readiness of the at least one emergency medical treatment and guidance apparatus and the at least one defibrillator comprises:
receiving an indication from the at least one defibrillator regarding whether the at least one defibrillator has passed a self-diagnostic test.

12. The deployment station of claim 1, wherein determining readiness of the at least one emergency medical treatment and guidance apparatus and the at least one defibrillator comprises:
querying the database to determine if a status report indicates a use of the at least one emergency medical treatment and guidance apparatus.

13. The deployment station of claim 1, wherein the at least one defibrillator comprises an automated external defibrillator (AED) and the AED comprises electronic circuitry for performing a self-diagnostic capability to determine a status of the AED, and wherein the determined status is transmitted to the at least one processor of the deployment station and the readiness of the at least one emergency medical treatment and guidance apparatus and the at least one defibrillator is based on the determined status of the AED.

14. The deployment station of claim 1, wherein the housing further comprises an access door mechanically hinged to the housing and lockable using a locking mechanism, and wherein the access door prevents access to the at least one emergency medical treatment and guidance apparatus and the at least one defibrillator when in a locked configuration and does not prevent access in an unlocked configuration.

15. The deployment station of claim 14, wherein the locking mechanism is unlocked when a user's identification has been verified with a list of authorized users stored in the database, the user's identification being represented by a passcode, an access card, or an RFID tag of the user.

16. The deployment station of claim 1, wherein the housing further comprises a charging system configured to charge the at least one emergency medical treatment and guidance apparatus or the at least one defibrillator, and wherein the at least one processor and memory is configured for:
receiving a battery status of the at least one emergency medical treatment and guidance apparatus or the at least one defibrillator, and
wherein the readiness is further based on the received battery status.

17. The deployment station of claim 1, wherein the housing further comprises an environmental controller configured to maintain a target temperature and a target humidity of an interior volume of the housing using a heater and a dehumidifier, respectively, wherein the at least one processor and memory is configured for:
receiving a measured temperature and a measured humidity of the interior volume of the housing, and
wherein the readiness is further based on the received temperature and the received humidity.

18. The deployment station of claim 17, wherein the readiness is further based on whether the measured temperature and the measured humidity are outside of target pre-determined ranges for the at least one emergency medical treatment and guidance apparatus and/or the at least one defibrillator.

19. The deployment station of claim 17, wherein the readiness is further based on whether the measured temperature and the measured humidity are outside of target pre-determined ranges for at least one pharmaceutical within the at least one emergency medical treatment and guidance apparatus.

20. The deployment station of claim 17, wherein the readiness is further based on whether the measured temperature and the measured humidity are outside of target pre-determined ranges for at least one electrode of the at least one defibrillator.

21. The deployment station of claim 1, wherein the housing further comprises a user interface configured to display a machine-readable code and provide an interactive query to a user.

22. The deployment station of claim 21, wherein the machine-readable code comprises a barcode or a QR code.

23. The deployment station of claim 21, wherein the machine-readable code represents the readiness of the at least one emergency medical treatment and guidance apparatus and the at least one defibrillator.

24. The deployment station of claim 21, wherein the machine-readable code encodes a hyperlink such that when imaged by a mobile device the hyperlink directs the mobile device to retrieve contact information from the database and display information related to whom to contact to obtain access to the deployment station.

25. The deployment station of claim 21, wherein the machine-readable code encodes a hyperlink such that when imaged by a mobile device the hyperlink directs the mobile device to retrieve connection information from the database and display information related to which devices are in network communication with the deployment station.

26. The deployment station of claim 25, wherein the devices related to the displayed information include the at least one emergency medical treatment and guidance apparatus, the at least one defibrillator, and the server.

27. The deployment station of claim 21, wherein the machine-readable code encodes a hyperlink such that when imaged by a mobile device the hyperlink directs the mobile device to retrieve readiness information from the database and display information related to the readiness of the at least one emergency medical treatment and guidance apparatus or the at least one defibrillator.

28. The deployment station of claim 21, wherein the interactive query is provided for guiding the user in determining which of the at least one emergency medical treatment and guidance apparatus and the at least one defibrillator is needed for medical treatment based on a condition of a patient.

29. The deployment station of claim 28, wherein the at least one processor and memory is further configured for recommending one of the at least one emergency medical treatment and guidance apparatus and/or one of the at least one defibrillator based on the query.

30. The deployment station of claim 1, wherein the housing further comprises a fire extinguisher and the at least one processor and memory is configured for:
receiving a fire extinguisher signal from at least one sensor of the fire extinguisher, the fire extinguisher signal representing that the fire extinguisher is present within the deployment station, and
wherein the readiness is further based on the fire extinguisher signal.

31. The deployment station of claim 1, wherein the housing further comprises:
a first mount located within the first storage section for releasably mounting the at least one emergency medical treatment and guidance apparatus within the first storage section; and
a second mount located within the second storage section for releasably mounting the at least one defibrillator within the second storage section.

32. The deployment station of claim 1, wherein each of the first storage section and the second storage section comprise an access door, each access door comprising a display for presenting a machine-readable code and a storage section readiness indicator, the display configured to display the machine-readable code encoding a hyperlink such that when imaged by a mobile device the hyperlink directs the mobile device to retrieve readiness information from the database and display information related to the readiness of the at least one emergency medical treatment and guidance apparatus contained within the first storage section and/or the readiness of the at least one defibrillator contained within the second storage section.

33. The deployment station of claim 1, wherein the housing further comprises a return chute for depositing used emergency medical treatment and guidance apparatuses and used defibrillators into a storage container within the housing of the deployment station.

34. The deployment station of claim 1, wherein the at least one processor and memory is further configured for:
reading each of at least one RFID tag of a used emergency medical treatment and guidance apparatus and/or a used defibrillator upon being returned to the deployment station; and
identifying the used emergency medical treatment and guidance apparatus and/or used defibrillator based on at least one of the at least one RFID tag.

35. The deployment station of claim 34, wherein the at least one processor and memory is further configured for:
transmitting information to the server that the used emergency medical treatment and guidance apparatus and/or the used defibrillator is located within the deployment station, and
wherein the server is configured to update location information within the database.

36. The deployment station of claim 1, wherein the at least one processor and memory is further configured for:
receiving information from the database representing expected RFID tags associated with a used emergency medical treatment and guidance apparatus and/or a used defibrillator upon being returned to the deployment station; and
determining whether all RFID tags are present based on comparing the read information of the RFID tags with the received information representing the expected RFID tags, and
wherein the readiness is based on whether all RFID tags are determined to be present.

37. The deployment station of claim 1, wherein the at least one processor and memory is further configured for:
measuring a weight of a used emergency medical treatment and guidance apparatus and/or a used defibrillator using a weight scale of the deployment station upon being returned to the deployment station; and
determining whether the weight is within a pre-determined range of an expected weight based on comparing the weight with an expected weight of the used emergency medical treatment and guidance apparatus and/or used defibrillator, and
wherein the readiness is based on whether the weight is determined to be within the pre-determined range.

38. The deployment station of claim 1, wherein the at least one processor and memory is further configured for:
querying the database to determine whether a used emergency medical treatment and guidance apparatus and/or a used defibrillator was used in a medical emergency upon being returned to the deployment station, and
wherein the readiness is based on whether used emergency medical treatment and guidance apparatus and/or used defibrillator was used in a medical emergency.

39. The deployment station of claim 38, wherein the determination of whether the used emergency medical treatment and guidance apparatus and/or used defibrillator was used in the medical emergency is performed by the server and determined based on a status report associated with the used emergency medical treatment and guidance apparatus and/or used defibrillator.

40. The deployment station of claim 1, wherein the at least one processor and memory is further configured for transmitting a signal representing the readiness to a mobile device such that the signal causes the mobile device to trigger a mobile alert indicating the readiness of the at least one emergency medical treatment and guidance apparatus and/or the at least one defibrillator.

41. The deployment station of claim 1, wherein the first storage section comprises the at least one emergency medical treatment and guidance apparatus containing a plurality of medical supplies and the second storage section comprises the at least one defibrillator comprising electronic circuitry to administer electrotherapy.

42. The deployment station of claim 41, wherein the plurality of medical supplies include at least one tourniquet, at least one chest seal, and at least one pair of gloves, and the at least one emergency medical treatment and guidance apparatus is configured to provide an interactive query flow of medical instructions using at least some of the plurality of medical supplies.

43. The deployment station of claim 41, wherein the electronic circuitry to administer electrotherapy includes at least one electrode and at least one high-voltage capacitor to administer electrotherapy.

44. The deployment station of claim 41, wherein the at least one emergency medical treatment and guidance apparatus and the at least one defibrillator each comprise an individual readiness indicator.

45. The deployment station of claim 41, wherein the housing further comprises a camera, the camera being operable to obtain an image of individual readiness indicators of the at least one emergency medical treatment and guidance apparatus and/or the at least one defibrillator, the at least one processor operable to process the image to determine an imaged individual readiness of the respective at least one emergency medical treatment and guidance apparatus and/or the at least one defibrillator such that the readiness of the respective at least one emergency medical treatment and guidance apparatus and/or the at least one defibrillator is based on the imaged individual readiness.

46. The deployment station of claim 45, wherein the housing further comprises a light operable to illuminate the at least one emergency medical treatment and guidance apparatus and/or the at least one defibrillator when the camera obtains an image.

47. The deployment station of claim 41, wherein the housing further comprises a microphone and the at least one emergency medical treatment and guidance apparatus and/or the at least one defibrillator comprises a speaker and circuitry for transmitting an audible signal to the microphone.

48. The deployment station of claim 47, wherein the at least one processor and memory is further configured for:
receiving the audible signal from the microphone, the audible signal encoding an audible message about the readiness of the at least one emergency medical treatment and guidance apparatus and/or at least one defibrillator; and
decoding the audible message; and
wherein the readiness is further based on the decoded audible message.

49. The deployment station of claim 47, wherein the at least one processor and memory is further configured for:
receiving the audible signal from the microphone, the audible signal encoding an audible message about the usage of the at least one emergency medical treatment and guidance apparatus and/or at least one defibrillator;
decoding the audible message; and
transmitting a request to the server to update the database based on the decoded audible message.

50. The deployment station of claim 47, wherein the at least one processor and memory is further configured for:
receiving the audible signal from the microphone, the audible signal encoding an audible message about missing items of at least one emergency medical treatment and guidance apparatus and/or at least one defibrillator;
decoding the audible message; and
transmitting a request to the server to update the database based on the decoded audible message.

51. The deployment station of claim 47, wherein the audible signal is frequency encoded or encoded using a Morse code scheme.

52. The deployment station of claim 51, wherein a first frequency indicates that the at least one defibrillator is ready and a second frequency indicates that the at least one defibrillator is not ready.

53. The deployment station of claim 1, wherein the readiness indicator is a first readiness indicator and the deployment station further comprises a second readiness indicator disposed on the exterior surface of the housing.

54. The deployment station of claim 53, wherein updating the readiness indicator based on the determined readiness comprises:
updating the first readiness indicator based on a portion of the determined readiness relating to the determined readiness of a tablet device of the at least one emergency medical treatment and guidance apparatus; and
updating the second readiness indicator based on a remainder portion of the determined readiness.
